(19) United States
(12) Patent Application Publication
(10) Pub. No.: US 2021/0000906 A1
(43) Pub. Date: Jan. 7, 2021

(54) MODULATION OF HSD17B13 EXPRESSION

(71) Applicant: Ionis Pharmaceuticals, Inc., Carlsbad, CA (US)

(72) Inventors: Susan F. Murray, Poway, CA (US); Jiangwei Zhang, San Marcos, CA (US); Huynh-Hoa Bui, San Diego, CA (US)

(73) Assignee: Ionis Pharmaceuticals, Inc., Carlsbad, CA (US)

(21) Appl. No.: 16/982,733

(22) PCT Filed: Mar. 21, 2019
(86) PCT No.: PCT/US2019/023333

§ 371 Date: Sep. 21, 2020

Related U.S. Application Data
(60) Provisional application No. 62/646,338, filed on Mar. 21, 2018.

(51) Int. Cl. A61K 38/00; A61P 1/16; C12N 15/113

(52) U.S. Cl. CPC A61K 38/005; A61K 9/0019; C12N 15/1137; A61P 1/16

(57) ABSTRACT

Provided herein are methods, compounds, and compositions for reducing expression of HSD17B13 in a cell or individual. Such methods, compounds, and compositions are useful to treat, prevent, delay, or ameliorate a liver disease, metabolic disease, or cardiovascular disease or disorder, including but not limited to NASH, in an individual.

### SEQUENCE LISTING

The present application is being filed along with a Sequence Listing in electronic format. The Sequence Listing is provided as a file entitled BIOL0336WOSEQ_ST25.txt, created on Mar. 18, 2019 which is 120 Kb in size. The information in the electronic format of the sequence listing is incorporated herein by reference in its entirety.

### FIELD

Provided herein are methods, compounds, and compositions useful for reducing expression or activity of hydroxysteroid 17-beta dehydrogenase 13 (hereinafter referred to as HSD17B13) in an individual. Also, provided herein are methods, compounds, and compositions comprising HSD17B13 specific inhibitors, which can be useful in reducing HSD17B13-related diseases or conditions in an individual. Such methods, compounds, and compositions can be useful, for example, to treat, prevent, delay or ameliorate liver disease, metabolic disease, or cardiovascular disease in an individual.

### BACKGROUND

Nonalcoholic fatty liver diseases (NAFLDs) including NASH (nonalcoholic steatohepatitis) are considered to be hepatic manifestations of the metabolic syndrome (Marchesini G, et al. Hepatology 2003; 37: 917-923) and are characterized by the accumulation of triglycerides in the liver of patients without a history of excessive alcohol consumption. The majority of patients with NAFLD are obese or morbidly obese and have accompanying insulin resistance (Byrne C D and Targher G. J Hepatol 2015 April; 62(1S): S47-S64). The incidence of NAFLD/NASH has been rapidly increasing worldwide consistent with the increased prevalence of obesity, and is currently the most common chronic liver disease. Recently, the incidence of NAFLD and NASH was reported to be 46% and 12%, respectively, in a largely middle-aged population (Williams C D, et al. Gastroenterology 2011; 140: 124-131).

NAFLD is classified into simple steatosis, in which only hepatic steatosis is observed, and NASH, in which intralobular inflammation and ballooning degeneration of hepatocytes is observed along with hepatic steatosis. The proportion of patients with NAFLD who have NASH is still not clear but might range from 20-40%. NASH is a progressive disease and may lead to liver cirrhosis and hepatocellular carcinoma (Farrell G C and Larter C Z. Hepatology 2006; 43: S99-S112; Cohen J C, et al. Science 2011; 332: 1519-1523). Twenty percent of NASH patients are reported to develop cirrhosis, and 30-40% of patients with NASH cirrhosis experience liver-related death (McCullough A J. J Clin Gastroenterol 2006; 40 Suppl 1: S17-S29). Recently, NASH has become the third most common indication for liver transplantation in the United States (Charlton M R, et al. Gastroenterology 2011; 141: 1249-1253).

Currently, the principal treatment for NAFLD/NASH is lifestyle modification by diet and exercise. However, pharmacological therapy is indispensable because obese patients with NAFLD often have difficulty maintaining improved lifestyles.

### SUMMARY

Provided herein are compositions, compounds and methods for modulating expression of HSD17B13-associated with liver disease, metabolic disease, or cardiovascular diseases or disorders. A loss-of-function variant in HSD17B13 has been associated with a reduced risk of certain liver diseases. N Engl J Med 2018; 378:1096-106. In certain embodiments, these compositions, compounds and methods are for modulating the expression of HSD17B13. In certain embodiments, the HSD17B13 modulator is a HSD17B13-specific inhibitor. In certain embodiments, the HSD17B13-specific inhibitor decreases expression or activity of HSD17B13. In certain embodiments, HSD17B13-specific inhibitors include nucleic acids, proteins and small molecules. In certain embodiments, the HSD17B13-specific inhibitor is a nucleic acid. In certain embodiments, the HSD17B13-specific inhibitor comprises a modified oligonucleotide. In certain embodiments, the modified oligonucleotide can be single stranded or double stranded.

Certain embodiments are directed to compounds useful for inhibiting HSD17B13, which can be useful for treating, ameliorating, or slowing progression of a liver disease, metabolic disease, or cardiovascular disease or disorder. Certain embodiments relate to the novel findings of antisense inhibition of HSD17B13 resulting in improvement of symptoms or endpoints associated with liver disease, metabolic disease, or cardiovascular disease or disorder. Certain embodiments are directed to compounds useful in improving hepatic steatosis, liver fibrosis, triglyceride synthesis, lipid levels, hepatic lipids, ALT levels, NAFLD Activity Score (NAS), cholesterol levels, or triglyceride levels.

Certain embodiments are described in the numbered embodiments below:

Embodiment 1: A method of treating, preventing, delaying the onset, slowing the progression, or ameliorating a liver disease or disorder in an individual having, or at risk of having, a liver disease or disorder comprising administering an HSD17B13 specific inhibitor to the individual, thereby treating, preventing, delaying the onset, slowing the progression, or ameliorating the liver disease or disorder in the individual.
Embodiment 2: The method of embodiment 1, wherein the liver disease or disorder is fatty liver disease, chronic liver disease, liver cirrhosis, hepatic steatosis, steatohepatitis, nonalcoholic fatty liver disease (NAFLD), or nonalcoholic steatohepatitis (NASH).
Embodiment 3: The method of embodiments 1 or 2, wherein the HSD17B13 specific inhibitor reduces or improves hepatic steatosis, liver fibrosis, triglyceride synthesis, lipid levels, hepatic lipids, ALT levels, NAFLD Activity Score (NAS), cholesterol levels, or triglyceride levels.
Embodiment 4: A method of inhibiting expression or activity of HSD17B13 in a cell comprising contacting the cell with an HSD17B13 specific inhibitor, thereby inhibiting expression or activity of HSD17B13 in the cell.
Embodiment 5: The method of embodiment 4, wherein the cell is a hepatocyte.
Embodiment 6: The method of embodiment 5, wherein the cell is in an individual.
Embodiment 7: The method of embodiment 6, wherein the individual has, or is at risk of having liver disease, fatty liver disease, chronic liver disease, liver cirrhosis, hepatic steatosis, steatohepatitis, nonalcoholic fatty liver disease (NAFLD), or nonalcoholic steatohepatitis (NASH).
Embodiment 8: The method of any preceding embodiment, wherein the individual is human.
Embodiment 9: The method of any preceding embodiment, wherein the HSD17B13 specific inhibitor is selected from a nucleic acid, a polypeptide, an antibody, and a small molecule.
Embodiment 10: The method of any preceding embodiment, wherein the HSD17B13 specific inhibitor comprises a modified oligonucleotide, wherein the modified oligonucleotide has a nucleobase sequence complementary to any one of SEQ ID NOs: 1-6.
Embodiment 11: The method of embodiment 10, wherein the modified oligonucleotide is single-stranded.
Embodiment 12: The method of embodiment 10, wherein the modified oligonucleotide is double-stranded.
Embodiment 13: The method of any one of embodiments 10-12, wherein the modified oligonucleotide consists of 12 to 30 linked nucleosides.
Embodiment 14: The method of embodiment 13, wherein at least one of the nucleosides comprise a modified sugar moiety.
Embodiment 15: The method of embodiment 13 or embodiment 14, wherein at least one of the nucleosides comprise a modified nucleobase.
Embodiment 16: The method of any one of embodiments 13-15, wherein at least one internucleoside linkage of the modified oligonucleotide is a modified internucleoside linkage.
Embodiment 17: The method of embodiment 14, wherein the modified sugar is a bicyclic sugar or 2′-O-methyoxyethyl.
Embodiment 18: The method of embodiment 14, wherein the modified sugar comprises a 4′-CH(CH₃)—O-2′ bridge or a 4′-(CH₂)ₙ—O-2′ bridge, wherein n is 1 or 2.
Embodiment 19: The method of embodiment 15, wherein the modified nucleobase is a 5-methylcytosine.
Embodiment 20: The method of embodiment 16, wherein the at least one modified internucleoside linkage is a phosphorothioate internucleoside linkage.
Embodiment 21: The method of any one of embodiments 10-20, wherein the modified oligonucleotide has:
a gap segment consisting of linked deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar.
   Embodiment 22: The method of any of the preceding embodiments, wherein the HSD17B13 specific inhibitor is administered parenterally.
   Embodiment 23: The method of embodiment 18, wherein the compound is administered parenterally by subcutaneous or intravenous administration.
   Embodiment 24: The method of any of the preceding embodiments, comprising co-administering the compound and at least one additional therapy.
   Embodiment 25: Use of an HSD17B13 specific inhibitor for the manufacture or preparation of a medicament for treating a liver disease or disorder.
   Embodiment 26: Use of an HSD17B13 specific inhibitor for the treatment of a liver disease or disorder.
   Embodiment 27: The use of embodiments 25 or 26, wherein the liver disease or disorder is fatty liver disease, chronic liver disease, liver cirrhosis, hepatic steatosis, steatohepatitis, nonalcoholic fatty liver disease (NAFLD), or nonalcoholic steatohepatitis (NASH).
   Embodiment 28: The use of any of embodiments 25-27, wherein the HSD17B13 specific inhibitor reduces or improves hepatic steatosis, liver fibrosis, triglyceride synthesis, lipid levels, hepatic lipids, ALT levels, NAFLD

Activity Score (NAS), cholesterol levels, or triglyceride levels.

Embodiment 29: The use of any of embodiments 25-28, wherein the HSD17B13 specific inhibitor is selected from a nucleic acid, a polypeptide, an antibody, and a small molecule.
Embodiment 30: The use of any of embodiments 25-29, wherein the HSD17B13 specific inhibitor comprises a modified oligonucleotide, wherein the modified oligonucleotide has a nucleobase sequence complementary to any one of SEQ ID NOs: 1-6.
Embodiment 31: The use of embodiment 30, wherein the modified oligonucleotide is single-stranded.
Embodiment 32: The use of embodiment 30, wherein the modified oligonucleotide is double-stranded
Embodiment 33: The use of any one of embodiments 30-32, wherein the modified oligonucleotide consists of 12 to 30 linked nucleosides.
Embodiment 34: The use of embodiment 33, wherein at least one of the nucleosides comprise a modified sugar moiety.
Embodiment 35: The use of embodiment 33 or embodiment 34, wherein at least one of the nucleosides comprise a modified nucleobase.
Embodiment 36: The use of any one of embodiments 33-35, wherein at least one internucleoside linkage of the modified oligonucleotide is a a modified internucleoside linkage.
Embodiment 37: The method of embodiment 34, wherein the modified sugar is a bicyclic sugar or 2′-O-methyoxyethyl.
Embodiment 38: The method of embodiment 34, wherein the modified sugar comprises a 4′-CH(CH₃)—O-2′ bridge or a 4′-(CH₂)ₙ—O-2′ bridge, wherein n is 1 or 2.
Embodiment 39: The method of embodiment 35, wherein the modified nucleobase is a 5-methylcytosine.
Embodiment 40: The method of embodiment 36, wherein the at least one modified internucleoside linkage is a phosphorothioate internucleoside linkage.
Embodiment 41: The use of any one of embodiments 30-40, wherein the modified oligonucleotide has:
a gap segment consisting of linked deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar.
   Embodiment 42: A method comprising administering an HSD17B13 specific inhibitor to an individual.
   Embodiment 43: The method of embodiment 42, wherein the individual has a liver disease or is at risk for developing a liver disease.
   Embodiment 44: The method of embodiment 43, wherein the liver disease is selected from fatty liver disease, chronic liver disease, liver cirrhosis, hepatic steatosis, steatohepatitis, nonalcoholic fatty liver disease (NAFLD), and nonalcoholic steatohepatitis (NASH).
   Embodiment 45: The method of embodiments 43 or 44, wherein a therapeutic amount of the HSD17B13 specific inhibitor is administered to the individual.
   Embodiment 46: The method any of embodiments 43-45, wherein the administration of the HSD17B13 specific inhibitor results in the prevention, delay, slowed progression, and/or amelioration of at least one symptom of the liver disease.
   Embodiment 47: The method of any of embodiments 42-46, wherein the administration of the HSD17B13 specific inhibitor reduces, improves, or regulates hepatic steatosis, liver fibrosis, triglyceride synthesis, lipid levels, hepatic lipids, ALT levels, NAFLD Activity Score (NAS), cholesterol levels, or triglyceride levels.
   Embodiment 48: A method comprising contacting a cell with an HSD17B13 specific inhibitor.
   Embodiment 49: The method of embodiment 48, wherein expression of HSD17B13 in the cell is reduced.
   Embodiment 50: The method of claim 48 or 49, wherein the cell is a hepatocyte.
   Embodiment 51: The method of embodiment 50, wherein the cell is in an individual.
   Embodiment 52: The method of embodiment 51, wherein the individual has, or is at risk of having liver disease, fatty liver disease, chronic liver disease, liver cirrhosis, hepatic steatosis, steatohepatitis, nonalcoholic fatty liver disease (NAFLD), or nonalcoholic steatohepatitis (NASH).
   Embodiment 53: The method of any preceding embodiment, wherein the individual is human.
   Embodiment 54: The method of any preceding embodiment, wherein the HSD17B13 specific inhibitor comprises or consists of a nucleic acid, a polypeptide, an antibody, or a small molecule.
   Embodiment 55: The method of any preceding embodiment, wherein the HSD17B13 specific inhibitor comprises a modified oligonucleotide, wherein the modified oligonucleotide has a nucleobase sequence complementary to any one of SEQ ID NOs: 1-6.
   Embodiment 56: The method of embodiment 55, wherein the modified oligonucleotide is single-stranded.
   Embodiment 57: The method of embodiment 55, wherein the modified oligonucleotide is double-stranded.
   Embodiment 58: The method of any of embodiments 55-57, wherein the modified oligonucleotide consists of 12 to 30 linked nucleosides.
   Embodiment 59: The method of embodiment 58, wherein at least one nucleoside of the modified oligonucleotide comprises a modified sugar moiety.
   Embodiment 60: The method of embodiment 59, wherein the modified sugar moiety is a bicyclic sugar moiety or a sugar moiety comprising a 2′-O-methyoxyethyl.
   Embodiment 61: The method of embodiment 59, wherein the modified sugar comprises a 4′-CH(CH₃)—O-2′ bridge or a 4′-(CH₂)ₙ—O-2′ bridge, wherein n is 1 or 2.
   Embodiment 62: The method of any of embodiments 58-61, wherein at least one nucleoside of the modified oligonucleotide comprises a modified nucleobase.
   Embodiment 63: The method of embodiment 62, wherein the modified nucleobase is a 5-methylcytosine.
   Embodiment 64: The method of any one of embodiments 58-63, wherein at least one internucleoside linkage of the modified oligonucleotide is a modified internucleoside linkage.
   Embodiment 65: The method of embodiment 64, wherein the at least one modified internucleoside linkage is a phosphorothioate internucleoside linkage.
   Embodiment 66: The method of any one of embodiments 55-65, wherein the modified oligonucleotide has:
a gap segment consisting of linked deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar.
   Embodiment 67: The method of any of the preceding embodiments, wherein the HSD17B13 specific inhibitor is administered parenterally.
   Embodiment 68: The method of embodiment 67, wherein the HSD17B13 specific inhibitor is administered parenterally by subcutaneous or intravenous administration.
   Embodiment 69: The method of any of the preceding embodiments, comprising co-administering the HSD17B13 specific inhibitor and at least one additional therapy.
   Embodiment 70: Use of an HSD17B13 specific inhibitor for the manufacture or preparation of a medicament for treating a liver disease or disorder.
   Embodiment 71: Use of an HSD17B13 specific inhibitor for the treatment of a liver disease or disorder.
   Embodiment 72: The use of embodiments 70 or 71, wherein the liver disease or disorder is fatty liver disease, chronic liver disease, liver cirrhosis, hepatic steatosis, steatohepatitis, nonalcoholic fatty liver disease (NAFLD), or nonalcoholic steatohepatitis (NASH).
   Embodiment 73: The use of any of embodiments 70-72, wherein the compound reduces, improves, or regulates hepatic steatosis, liver fibrosis, triglyceride synthesis, lipid levels, hepatic lipids, ALT levels, NAFLD Activity Score (NAS), cholesterol levels, or triglyceride levels.
   Embodiment 74: The use of any of embodiments 70-73, wherein the HSD17B13 specific inhibitor comprises a nucleic acid, a polypeptide, an antibody, or a small molecule.
   Embodiment 75: The use of any of embodiments 70-74, wherein the HSD17B13 specific inhibitor comprises a modified oligonucleotide, wherein the modified oligonucleotide has a nucleobase sequence complementary to any one of SEQ ID NOs: 1-6.
   Embodiment 76: The use of embodiment 75, wherein the compound is single-stranded.
   Embodiment 77: The use of embodiment 75, wherein the compound is double-stranded
   Embodiment 78: The use of any one of embodiments 75-77, wherein the modified oligonucleotide consists of 12 to 30 linked nucleosides.
   Embodiment 79: The use of embodiment 78, wherein at least one of the nucleosides comprise a modified sugar moiety.
   Embodiment 80: The use of embodiment 78 or embodiment 79, wherein at least one of the nucleosides comprise a modified nucleobase.
   Embodiment 81: The use of any one of embodiments 78-80, wherein at least one internucleoside linkage of the modified oligonucleotide is a modified internucleoside linkage.
   Embodiment 82: The method of embodiment 79, wherein the modified sugar is a bicyclic sugar or 2′-O-methyoxyethyl.
   Embodiment 83: The method of embodiment 79, wherein the modified sugar comprises a 4′-CH(CH₃)—O-2′ bridge or a 4′-(CH₂)ₙ—O-2′ bridge, wherein n is 1 or 2.
   Embodiment 84: The method of embodiment 80, wherein the modified nucleobase is a 5-methylcytosine.
   Embodiment 85: The method of embodiment 81, wherein the at least one modified internucleoside linkage is a phosphorothioate internucleoside linkage.
   Embodiment 86: The use of any one of embodiments 75-85, wherein the modified oligonucleotide has:
a gap segment consisting of linked deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ISIS/IONIS number (ISIS/ION #) indicate a combination of nucleobase sequence, chemical modification, and motif.

Unless otherwise indicated, the following terms have the following meanings:

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-O-methoxyethyl” (also 2′-MOE and 2′-O(CH₂)₂—OCH₃) refers to an O-methoxy-ethyl modification at the 2′ position of a furanosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“3′ target site” refers to the nucleotide of a target nucleic acid which is complementary to the 3′-most nucleotide of a particular compound.

“5′ target site” refers to the nucleotide of a target nucleic acid which is complementary to the 5′-most nucleotide of a particular compound.

“5-methylcytosine” means a cytosine with a methyl group attached to the 5 position.

“About” means within ±10% of a value. For example, if it is stated, “the compounds affected about 70% inhibition of HSD17B13”, it is implied that HSD17B13 levels are inhibited within a range of 60% and 80%.

“Administration” or “administering” refers to routes of introducing a compound or composition provided herein to an individual to perform its intended function. An example of a route of administration that can be used includes, but is not limited to parenteral administration, such as subcutaneous, intravenous, or intramuscular injection or infusion.

“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

“Amelioration” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable and/or measurable activity attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target.

“Antisense compound” means a compound comprising an oligonucleotide and optionally one or more additional features, such as a conjugate group or terminal group. Examples of antisense compounds include single-stranded and double-stranded compounds, such as, oligonucleotides, ribozymes, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.

“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell-targeting moiety” means a conjugate group or portion of a conjugate group that is capable of binding to a particular cell type or particular cell types.

“cEt” or “constrained ethyl” means a bicyclic furanosyl sugar moiety comprising a bridge connecting the 4′-carbon and the 2′-carbon, wherein the bridge has the formula: 4′-CH(CH₃)—O-2′.

“Chemical modification” in a compound describes the substitutions or changes through chemical reaction, of any of the units in the compound. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group of atoms that is cleaved under physiological conditions, for example, inside a cell, an animal, or a human.

“Complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (^{m}C) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Conjugate group” means a group of atoms that is attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Designing” or “Designed to” refer to the process of designing a compound that specifically hybridizes with a selected nucleic acid molecule.

“Diluent” means an ingredient in a composition that lacks pharmacological activity, but is pharmaceutically necessary or desirable. For example, the diluent in an injected composition can be a liquid, e.g. saline solution.

“Differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

“Dose” means a specified quantity of a compound or pharmaceutical agent provided in a single administration, or in a specified time period. In certain embodiments, a dose may be administered in two or more boluses, tablets, or injections. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in an individual. In other embodiments, the compound or pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Double-stranded compound” means a compound comprising two oligomeric compounds that are complementary to each other and form a duplex, and wherein one of the two said oligomeric compounds comprises an oligonucleotide.

“HSD17B13” means hydroxysteroid 17-beta dehydrogenase 13 and refers to any nucleic acid of HSD17B13. For example, in certain embodiments, HSD17B13 includes a DNA sequence encoding HSD17B13, an RNA sequence transcribed from DNA encoding HSD17B13 (including genomic DNA comprising introns and exons). The target may be referred to in either upper or lower case.

“HSD17B13-specific inhibitor” refers to any agent capable of specifically inhibiting HSD17B13 expression or activity at the molecular level. For example, HSD17B13-specific inhibitors include nucleic acids (including antisense compounds), peptides, antibodies, small molecules, and other agents capable of inhibiting the expression or activity of HSD17B13.

“Effective amount” means the amount of compound sufficient to effectuate a desired physiological outcome in an individual in need of the compound. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information is converted into structures present and operating in a cell. Such structures include, but are not limited to the products of transcription and translation.

“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Hybridization” means annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

“Immediately adjacent” means there are no intervening elements between the immediately adjacent elements of the same kind (e.g. no intervening nucleobases between the immediately adjacent nucleobases).

“Individual” means a human or non-human animal selected for treatment or therapy.

“Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“Lengthened oligonucleotides” are those that have one or more additional nucleosides relative to an oligonucleotide disclosed herein, e.g. a parent oligonucleotide.

“Linked nucleosides” means adjacent nucleosides linked together by an internucleoside linkage.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Modulating” refers to changing or adjusting a feature in a cell, tissue, organ or organism. For example, modulating HSD17B13 can mean to increase or decrease the level of HSD17B13 in a cell, tissue, organ or organism. A “modulator” effects the change in the cell, tissue, organ or organism. For example, a compound can be a modulator of HSD17B13 that decreases the amount of HSD17B13 in a cell, tissue, organ or organism.

“MOE” means methoxyethyl.

“Monomer” refers to a single unit of an oligomer. Monomers include, but are not limited to, nucleosides and nucleotides.

“Motif” means the pattern of unmodified and/or modified sugar moieties, nucleobases, and/or internucleoside linkages, in an oligonucleotide.

“Natural” or “naturally occurring” means found in nature.

“Non-bicyclic modified sugar” or “non-bicyclic modified sugar moiety” means a modified sugar moiety that comprises a modification, such as a substituent, that does not form a bridge between two atoms of the sugar to form a second ring.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase, and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric compound” means a compound comprising a single oligonucleotide and optionally one or more additional features, such as a conjugate group or terminal group.

“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification.

“Parent oligonucleotide” means an oligonucleotide whose sequence is used as the basis of design for more oligonucleotides of similar sequence but with different lengths, motifs, and/or chemistries. The newly designed oligonucleotides may have the same or overlapping sequence as the parent oligonucleotide.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Pharmaceutically acceptable carrier or diluent” means any substance suitable for use in administering to an individual. For example, a pharmaceutically acceptable carrier can be a sterile aqueous solution, such as PBS or water-for-injection.

“Pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

“Pharmaceutical agent” means a compound that provides a therapeutic benefit when administered to an individual.

“Pharmaceutical composition” means a mixture of substances suitable for administering to an individual. For example, a pharmaceutical composition may comprise one or more compounds or salt thereof and a sterile aqueous solution.

“Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Prevent” refers to delaying or forestalling the onset, development or progression of a disease, disorder, or condition for a period of time from minutes to indefinitely.

“Prodrug” means a compound in a form outside the body which, when administered to an individual, is metabolized to another form within the body or cells thereof. In certain embodiments, the metabolized form is the active, or more active, form of the compound (e.g., drug). Typically conversion of a prodrug within the body is facilitated by the action of an enzyme(s) (e.g., endogenous or viral enzyme) or chemical(s) present in cells or tissues, and/or by physiologic conditions.

“Reduce” means to bring down to a smaller extent, size, amount, or number.

“RefSeq No.” is a unique combination of letters and numbers assigned to a sequence to indicate the sequence is for a particular target transcript (e.g., target gene). Such sequence and information about the target gene (collectively, the gene record) can be found in a genetic sequence database. Genetic sequence databases include the NCBI Reference Sequence database, GenBank, the European Nucleotide Archive, and the DNA Data Bank of Japan (the latter three forming the International Nucleotide Sequence Database Collaboration or INSDC).

“Region” is defined as a portion of the target nucleic acid having at least one identifiable structure, function, or characteristic.

“RNAi compound” means an antisense compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Segments” are defined as smaller or sub-portions of regions within a nucleic acid.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded” in reference to a compound means the compound has only one oligonucleotide. “Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Sites,” are defined as unique nucleobase positions within a target nucleic acid.

“Specifically hybridizable” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.

“Specifically inhibit” a target nucleic acid means to reduce or block expression of the target nucleic acid while exhibiting fewer, minimal, or no effects on non-target nucleic acids reduction and does not necessarily indicate a total elimination of the target nucleic acid's expression.

“Standard cell assay” means assay(s) described in the Examples and reasonable variations thereof

“Standard in vivo experiment” means the procedure(s) described in the Example(s) and reasonable variations thereof.

“Sugar moiety” means an unmodified sugar moiety or a modified sugar moiety. “Unmodified sugar moiety” or “unmodified sugar” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

“Synergy” or “synergize” refers to an effect of a combination that is greater than additive of the effects of each component alone at the same doses.

“Target gene” refers to a gene encoding a target.

“Targeting” means specific hybridization of a compound that to a target nucleic acid in order to induce a desired effect.

“Target nucleic acid,” “target RNA,” “target RNA transcript” and “nucleic acid target” all mean a nucleic acid capable of being targeted by compounds described herein.

“Target region” means a portion of a target nucleic acid to which one or more compounds is targeted.

“Target segment” means the sequence of nucleotides of a target nucleic acid to which a compound described herein is targeted. “5′ target site” refers to the 5′-most nucleotide of a target segment. “3′ target site” refers to the 3′-most nucleotide of a target segment.

“Terminal group” means a chemical group or group of atoms that is covalently linked to a terminus of an oligonucleotide.

“Therapeutically effective amount” means an amount of a compound, pharmaceutical agent, or composition that provides a therapeutic benefit to an individual.

“Treat” refers to administering a compound or pharmaceutical composition to an individual in order to effect an alteration or improvement of a disease, disorder, or condition in the individual.

Certain Embodiments

Certain embodiments provide methods, compounds, and compositions for modulating a liver disease, metabolic disease, or cardiovascular disease condition, or a symptom thereof, in an individual by administering the compound or composition to the individual, wherein the compound or composition comprises a HSD17B13 modulator. Modulation of HSD17B13 can lead to a decrease of HSD17B13 level or expression in order to treat, prevent, ameliorate or delay a liver disease, metabolic disease, or cardiovascular disease or disorder, or a symptom thereof. In certain embodiments, the HSD17B13 modulator is a HSD17B13-specific inhibitor. In certain embodiments, HSD17B13-specific inhibitors are nucleic acids (including antisense compounds), single-stranded oligonucleotides, double-stranded oligonucleotides including but not limited to siRNA, peptides, antibodies, small molecules, and other agents capable of inhibiting the expression or activity of HSD17B13. In certain embodiments, the individual is human.

Certain embodiments disclosed herein provide compounds or compositions comprising a HSD17B13 modulator. Such compounds or compositions are useful to treat, prevent, ameliorate or delay a liver disease, metabolic disease, or cardiovascular disease or disorder, or a symptom thereof. In certain embodiments, the compound comprises a HSD17B13-specific inhibitor. In certain embodiments, the HSD17B13-specific inhibitor is a nucleic acid, polypeptide, antibody, small molecules, or other agent capable of inhibiting the expression or activity of HSD17B13. In certain embodiments, the HSD17B13-specific inhibitor is a nucleic acid targeting HSD17B13. In certain embodiments, the nucleic acid is single stranded. In certain embodiments, the nucleic acid is double stranded. In certain embodiments, the compound or composition comprises an antisense compound. In any of the foregoing embodiments, the compound or composition comprises an oligomeric compound. In certain embodiments, the compound or composition comprises an oligonucleotide targeting HSD17B13. In certain embodiments, the oligonucleotide is single stranded. In certain embodiments, the compound comprises deoxyribonucleotides. In certain embodiments, the compound comprises ribonucleotides and is double-stranded. In certain embodiments, the oligonucleotide is a modified oligonucleotide. In certain embodiments, the modified oligonucleotide is single stranded. In certain embodiments, the HSD17B13-specific inhibitor is a double-stranded siRNA.

In any of the foregoing embodiments, the compound can comprise a modified oligonucleotide consisting of 8 to 80, 10 to 30, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked nucleosides.

In certain embodiments, at least one internucleoside linkage of said modified oligonucleotide is a modified internucleoside linkage. In certain embodiments, at least one internucleoside linkage is a phosphorothioate internucleoside linkage. In certain embodiments, the internucleoside linkages are phosphorothioate linkages and phosphate ester linkages.

In certain embodiments, any of the foregoing oligonucleotides comprises at least one modified sugar. In certain embodiments, at least one modified sugar comprises a 2′-O-methoxyethyl group. In certain embodiments, at least one modified sugar is a bicyclic sugar, such as a 4′-CH(CH₃)—O-2′ group, a 4′-CH₂—O-2′ group, or a 4′-(CH₂)₂—O-2′group.

In certain embodiments, at least one nucleoside of said modified oligonucleotide comprises a modified nucleobase. In certain embodiments, the modified nucleobase is a 5-methylcytosine.

In certain embodiments, a compound or composition comprises a modified oligonucleotide comprising: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar. In certain embodiments, at least one internucleoside linkage is a phosphorothioate linkage. In certain embodiments, and at least one cytosine is a 5-methylcytosine.

In certain embodiments, the compounds or compositions disclosed herein further comprise a pharmaceutically acceptable carrier or diluent.

In certain embodiments, the compound or composition is co-administered with a second agent. In certain embodiments, the compound or composition and the second agent are administered concomitantly.

In certain embodiments, compounds and compositions described herein targeting HSD17B13 can be used in methods of inhibiting expression of HSD17B13 in a cell. In certain embodiments, compounds and compositions described herein targeting HSD17B13 can be used in methods of treating, preventing, delaying or ameliorating a liver disease, metabolic disease, or cardiovascular disease or disorder including, but not limited to, metabolic syndrome, liver disease, fatty liver disease, chronic liver disease, liver cirrhosis, hepatic steatosis, steatohepatitis, nonalcoholic fatty liver disease (NAFLD), and nonalcoholic steatohepatitis (NASH).

### Certain Indications

Certain embodiments provided herein relate to methods of inhibiting HSD17B13 expression or activity, which can be useful for treating, preventing, or ameliorating a disease associated with HSD17B13 in an individual, such as NASH, by administration of a compound or composition that targets HSD17B13. In certain embodiments, such a compound or composition comprises a HSD17B13-specific inhibitor. In certain embodiments, the compound comprises an antisense compound or an oligomeric compound targeted to HSD17B13. In certain embodiments, the compound comprises a modified oligonucleotide targeted to HSD17B13. In certain embodiments, the compound is a double-stranded siRNA targeted to HSD17B13.

In certain embodiments, a method of inhibiting expression or activity of HSD17B13 in a cell comprises contacting the cell with a compound or composition comprising a HSD17B13-specific inhibitor, thereby inhibiting expression or activity of HSD17B13 in the cell. In certain embodiments, the cell is a hepatocyte cell. In certain embodiments, the cell is in the liver. In certain embodiments, the cell is in the liver of an individual who has, or is at risk of having a disease, disorder, condition, symptom, or physiological marker associated with a liver disease, metabolic disease, or cardiovascular disease or disorder. In certain embodiments, the liver disease, metabolic disease, or cardiovascular disease or disorder is metabolic syndrome, liver disease, fatty liver disease, chronic liver disease, liver cirrhosis, hepatic steatosis, steatohepatitis, nonalcoholic fatty liver disease (NAFLD), and nonalcoholic steatohepatitis (NASH). In certain embodiments, the liver disease, metabolic disease, or cardiovascular disease or disorder is NASH. In certain embodiments, the HSD17B13-specific inhibitor is a nucleic acid, peptide, antibody, small molecule or other agent capable of inhibiting the expression or activity of the HSD17B13. In certain embodiments, the HSD17B13-specific inhibitor is an antisense compound or an oligomeric compound targeted to HSD17B13. In certain embodiments, the HSD17B13-specific inhibitor is oligonucleotide targeted to HSD17B13. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound comprising a modified oligonucleotide can be single-stranded. In certain embodiments, the compound comprising a modified oligonucleotide can be double-stranded. In certain embodiments, the compound is a double-stranded siRNA targeted to HSD17B13.

In certain embodiments, a method of treating, preventing, delaying the onset, slowing the progression, or ameliorating one or more diseases, disorders, conditions, symptoms or physiological markers associated with HSD17B13 comprises administering to the individual a compound or composition comprising a HSD17B13-specific inhibitor. In certain embodiments, a method of treating, preventing, delaying the onset, slowing the progression, or ameliorating a disease, disorder, condition, symptom, or physiological marker associated with a liver disease, metabolic disease, or cardiovascular disease or disorder in an individual comprises administering to the individual a compound or composition comprising a HSD17B13-specific inhibitor, thereby treating, preventing, delaying the onset, slowing the progression, or ameliorating the disease. In certain embodiments, the individual is identified as having, or at risk of having, the disease, disorder, condition, symptom or physiological marker. In certain embodiments, the liver disease, metabolic disease, or cardiovascular disease or disorder is metabolic syndrome, liver disease, fatty liver disease, chronic liver disease, liver cirrhosis, hepatic steatosis, steatohepatitis, nonalcoholic fatty liver disease (NAFLD), and nonalcoholic steatohepatitis (NASH). In certain embodiments, the liver disease, metabolic disease, or cardiovascular disease or disorder is NASH. In certain embodiments, the HSD17B13-specific inhibitor is administered to the individual parenterally. In certain embodiments, the parenteral administration is subcutaneous administration. In certain embodiments, the individual is human. In certain embodiments, the HSD17B13-specific inhibitor is a nucleic acid, peptide, antibody, small molecule or other agent capable of inhibiting the expression or activity of the HSD17B13. In certain embodiments, the HSD17B13-specific inhibitor is an antisense compound or an oligomeric compound targeted to HSD17B13. In certain embodiments, the HSD17B13-specific inhibitor is oligonucleotide targeted to HSD17B13. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound comprising a modified oligonucleotide can be single-stranded. In certain embodiments, the compound comprising a modified oligonucleotide can be double-stranded. In certain embodiments, the compound is a double-stranded siRNA targeted to HSD17B13.

In certain embodiments, a method of reducing, improving, or regulating hepatic steatosis, liver fibrosis, triglyceride synthesis, lipid levels, hepatic lipids, ALT levels, NAFLD Activity Score (NAS), cholesterol levels, or triglyceride levels, or a combination thereof, in an individual comprises administering to the individual a compound or composition comprising a HSD17B13-specific inhibitor. In certain embodiments, administering the compound or composition reduces, improves, or regulates *SPECIFIC ENDPOINT 1* in the individual. In certain embodiments, the individual is identified as having, or at risk of having a disease, disorder, condition, symptom, or physiological marker associated with a liver disease, metabolic disease, or cardiovascular disease or disorder. In certain embodiments, the liver disease, metabolic disease, or cardiovascular disease or disorder is metabolic syndrome, liver disease, fatty liver disease, chronic liver disease, liver cirrhosis, hepatic steatosis, steatohepatitis, nonalcoholic fatty liver disease (NAFLD), and nonalcoholic steatohepatitis (NASH). In certain embodiments, the liver disease, metabolic disease, or cardiovascular disease or disorder is NASH. In certain embodiments, the HSD17B13-specific inhibitor is administered to the individual parenterally. In certain embodiments, the parenteral administration is subcutaneous administration. In certain embodiments, the individual is human. In certain embodiments, the HSD17B13-specific inhibitor is a nucleic acid, peptide, antibody, small molecule or other agent capable of inhibiting the expression or activity of the HSD17B13. In certain embodiments, the HSD17B13-specific inhibitor is an antisense compound or an oligomeric compound targeted to HSD17B13. In certain embodiments, the HSD17B13-specific inhibitor is oligonucleotide targeted to HSD17B13. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound comprising a modified oligonucleotide can be single-stranded. In certain embodiments, the compound comprising a modified oligonucleotide can be double-stranded. In certain embodiments, the compound is a double-stranded siRNA targeted to HSD17B13.

Certain embodiments are drawn to compounds and compositions described herein for use in therapy. Certain embodiments are drawn to a compound or composition comprising a HSD17B13-specific inhibitor for use in treating, preventing, delaying the onset, slowing the progression, or ameliorating one or more diseases, disorders, conditions, symptoms or physiological markers associated with HSD17B13. Certain embodiments are drawn to a compound or composition for use in treating, preventing, delaying the onset, slowing the progression, or ameliorating a liver disease, metabolic disease, or cardiovascular disease or disorder, or a symptom or physiological marker thereof. In certain embodiments, the liver disease, metabolic disease, or cardiovascular disease or disorder is metabolic syndrome, liver disease, fatty liver disease, chronic liver disease, liver cirrhosis, hepatic steatosis, steatohepatitis, nonalcoholic fatty liver disease (NAFLD), and nonalcoholic steatohepatitis (NASH). In certain embodiments, the liver disease, metabolic disease, or cardiovascular disease or disorder is NASH. In certain embodiments, the HSD17B13-specific inhibitor is a nucleic acid, peptide, antibody, small molecule or other agent capable of inhibiting the expression or activity of the HSD17B13. In certain embodiments, the HSD17B13-specific inhibitor is an antisense compound or an oligomeric compound targeted to HSD17B13. In certain embodiments, the HSD17B13-specific inhibitor is oligonucleotide targeted to HSD17B13. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound comprising a modified oligonucleotide can be single-stranded. In certain embodiments, the compound comprising a modified oligonucleotide can be double-stranded. In certain embodiments, the compound is a double-stranded siRNA targeted to HSD17B13.

Certain embodiments are drawn to a compound or composition comprising a HSD17B13-specific inhibitor for use in reducing, improving, or regulating hepatic steatosis, liver fibrosis, triglyceride synthesis, lipid levels, hepatic lipids, ALT levels, NAFLD Activity Score (NAS), cholesterol levels, or triglyceride levels, or a combination thereof, in an individual. In certain embodiments, the compound or composition is provided for use in reducing, improving, or regulating hepatic steatosis in the individual. In certain embodiments, the compound or composition is provided for use in reducing, improving, or regulating liver fibrosis in the individual. In certain embodiments, the compound or composition is provided for use in reducing, improving, or regulating triglyceride synthesis in the individual. In certain embodiments, the compound or composition is provided for use in reducing, improving, or regulating lipid levels in the individual. In certain embodiments, the compound or composition is provided for use in reducing, improving, or regulating hepatic lipids in the individual. In certain embodiments, the compound or composition is provided for use in reducing, improving, or regulating ALT levels in the individual. In certain embodiments, the compound or composition is provided for use in reducing, improving, or regulating NAFLD Activity Score in the individual. In certain embodiments, the compound or composition is provided for use in reducing, improving, or regulating cholesterol levels in the individual. In certain embodiments, the compound or composition is provided for use in reducing, improving, or regulating triglyceride levels in the individual. In certain embodiments, the individual is identified as having, or at risk of having a disease, disorder, condition, symptom, or physiological marker associated with a liver disease, metabolic disease, or cardiovascular disease or disorder. In certain embodiments, the liver disease, metabolic disease, or cardiovascular disease or disorder is metabolic syndrome, liver disease, fatty liver disease, chronic liver disease, liver cirrhosis, hepatic steatosis, steatohepatitis, nonalcoholic fatty liver disease (NAFLD), and nonalcoholic steatohepatitis (NASH). In certain embodiments, the liver disease, metabolic disease, or cardiovascular disease or disorder is NASH. In certain embodiments, the individual is human. In certain embodiments, the HSD17B13-specific inhibitor is a nucleic acid, peptide, antibody, small molecule or other agent capable of inhibiting the expression or activity of the HSD17B13. In certain embodiments, the HSD17B13-specific inhibitor is an antisense compound or an oligomeric compound targeted to HSD17B13. In certain embodiments, the HSD17B13-specific inhibitor is oligonucleotide targeted to HSD17B13. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound comprising a modified oligonucleotide can be single-stranded. In certain embodiments, the compound comprising a modified oligonucleotide can be double-stranded. In certain embodiments, the compound is a double-stranded siRNA targeted to HSD17B13.

Certain embodiments are drawn to use of compounds or compositions described herein for the manufacture or preparation of a medicament for therapy. Certain embodiments are drawn to the use of a compound or composition as described herein in the manufacture or preparation of a medicament for treating, preventing, delaying the onset, slowing the progression, or ameliorating one or more diseases, disorders, conditions, symptoms or physiological markers associated with HSD17B13. In certain embodiments, the compound or composition as described herein is used in the manufacture or preparation of a medicament for treating, ameliorating, delaying or preventing a liver disease, metabolic disease, or cardiovascular disease or disorder, or a symptom or physiological marker thereof. In certain embodiments, the liver disease, metabolic disease, or cardiovascular disease or disorder is metabolic syndrome, liver disease, fatty liver disease, chronic liver disease, liver cirrhosis, hepatic steatosis, steatohepatitis, nonalcoholic fatty liver disease (NAFLD), and nonalcoholic steatohepatitis (NASH). In certain embodiments, the liver disease, metabolic disease, or cardiovascular disease or disorder is NASH. In certain embodiments, the compound or composition comprises a nucleic acid, peptide, antibody, small molecule or other agent capable of inhibiting the expression or activity of the HSD17B13. In certain embodiments, the compound or composition comprises an antisense compound or an oligomeric compound targeted to HSD17B13. In certain embodiments, the compound or composition comprises an oligonucleotide targeted to HSD17B13. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound or composition comprising a modified oligonucleotide can be single-stranded. In certain embodiments, the compound or composition comprising a modified oligonucleotide can be double-stranded. In certain embodiments, the compound is a double-stranded siRNA targeted to HSD17B13.

Certain embodiments are drawn to the use of a compound or composition for the manufacture or preparation of a medicament for reducing, improving, or regulating hepatic steatosis, liver fibrosis, triglyceride synthesis, lipid levels, hepatic lipids, ALT levels, NAFLD Activity Score (NAS), cholesterol levels, or triglyceride levels, or a combination thereof, in an individual having or at risk of having a liver disease, metabolic disease, or cardiovascular disease or disorder. Certain embodiments are drawn to use of a compound or composition in the manufacture or preparation of a medicament for reducing, improving, or regulating hepatic steatosis in the individual. Certain embodiments are drawn to use of a compound or composition in the manufacture or preparation of a medicament for reducing, improving, or regulating liver fibrosis in the individual. Certain embodiments are drawn to use of a compound or composition in the manufacture or preparation of a medicament for reducing, improving, or regulating triglyceride synthesis in the individual. Certain embodiments are drawn to use of a compound or composition in the manufacture or preparation of a medicament for reducing, improving, or regulating lipid levels in the individual. Certain embodiments are drawn to use of a compound or composition in the manufacture or preparation of a medicament for reducing, improving, or regulating hepatic lipids in the individual. Certain embodiments are drawn to use of a compound or composition in the manufacture or preparation of a medicament for reducing, improving, or regulating ALT levels in the individual. Certain embodiments are drawn to use of a compound or composition in the manufacture or preparation of a medicament for reducing, improving, or regulating NAFLD Activity Score in the individual. Certain embodiments are drawn to use of a compound or composition in the manufacture or preparation of a medicament for reducing, improving, or regulating cholesterol levels in the individual. Certain embodiments are drawn to use of a compound or composition in the manufacture or preparation of a medicament for reducing, improving, or regulating triglyceride levels in the individual. In certain embodiments, the compound or composition comprises a nucleic acid, peptide, antibody, small molecule or other agent capable of inhibiting the expression or activity of the HSD17B13. In certain embodiments, the compound or composition comprises an antisense compound or an oligomeric compound targeted to HSD17B13. In certain embodiments, the compound or composition comprises an oligonucleotide targeted to HSD17B13. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound or composition comprising a modified oligonucleotide can be single-stranded. In certain embodiments, the compound or composition comprising a modified oligonucleotide can be double-stranded. In certain embodiments, the compound is a double-stranded siRNA targeted to HSD17B13.

In any of the foregoing methods or uses, the compound or composition can comprise an antisense compound targeted to HSD17B13. In certain embodiments, the compound comprises an oligonucleotide, for example an oligonucleotide consisting of 8 to 80 linked nucleosides, 10 to 30 linked nucleosides, 12 to 30 linked nucleosides, or 20 linked nucleosides. In certain embodiments, the oligonucleotide comprises at least one modified internucleoside linkage, at least one modified sugar and/or at least one modified nucleobase. In certain embodiments, the modified internucleoside linkage is a phosphorothioate internucleoside linkage, the modified sugar is a bicyclic sugar or a 2′-O-methoxyethyl, and the modified nucleobase is a 5-methylcytosine. In certain embodiments, the modified oligonucleotide comprises a gap segment consisting of linked deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar. In certain embodiments, the compound is an antisense compound or oligomeric compound. In certain embodiments, the compound is single-stranded. In certain embodiments, the compound is double-stranded. In certain embodiments, the modified oligonucleotide is 12 to 30 linked nucleosides in length. In certain embodiments, the compounds or compositions disclosed herein further comprise a pharmaceutically acceptable carrier or diluent.

In any of the foregoing methods or uses, the compound or composition comprises or consists of a modified oligonucleotide 12 to 30 linked nucleosides in length, wherein the modified oligonucleotide comprises:

a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar.

In any of the foregoing methods or uses, the compound or composition can be administered parenterally. For example, in certain embodiments the compound or composition can be administered through injection or infusion. Parenteral administration includes subcutaneous administration, intravenous administration, intramuscular administration, intraarterial administration, intraperitoneal administration, or intracranial administration. In certain embodiments, the parenteral administration is subcutaneous administration. In certain embodiments, the compound or composition is co-administered with a second agent. In certain embodiments, the compound or composition and the second agent are administered concomitantly.

### Certain Compounds

In certain embodiments, compounds described herein are antisense compounds. In certain embodiments, the antisense compound comprises or consists of an oligomeric compound. In certain embodiments, the oligomeric compound comprises a modified oligonucleotide. In certain embodiments, the modified oligonucleotide has a nucleobase sequence complementary to that of a target nucleic acid.

In certain embodiments, a compound described herein comprises or consists of a modified oligonucleotide. In certain embodiments, the modified oligonucleotide has a nucleobase sequence complementary to that of a target nucleic acid.

In certain embodiments, a compound or antisense compound is single-stranded. Such a single-stranded compound or antisense compound comprises or consists of an oligomeric compound. In certain embodiments, such an oligomeric compound comprises or consists of an oligonucleotide. In certain embodiments, the oligonucleotide is an antisense oligonucleotide. In certain embodiments, the oligonucleotide is modified. In certain embodiments, the oligonucleotide of a single-stranded antisense compound or oligomeric compound comprises a self-complementary nucleobase sequence.

In certain embodiments, compounds are double-stranded. Such double-stranded compounds comprise a first modified oligonucleotide having a region complementary to a target nucleic acid and a second modified oligonucleotide having a region complementary to the first modified oligonucleotide. In certain embodiments, the modified oligonucleotide is an RNA oligonucleotide. In such embodiments, the thymine nucleobase in the modified oligonucleotide is replaced by a uracil nucleobase. In certain embodiments, compound comprises a conjugate group. In certain embodiments, each modified oligonucleotide is 12-30 linked nucleosides in length.

In certain embodiments, compounds are double-stranded. Such double-stranded compounds comprise a first oligomeric compound having a region complementary to a target nucleic acid and a second oligomeric compound having a region complementary to the first oligomeric compound. The first oligomeric compound of such double stranded compounds typically comprises or consists of a modified oligonucleotide. The oligonucleotide of the second oligomeric compound of such double-stranded compound may be modified or unmodified. The oligomeric compounds of double-stranded compounds may include non-complementary overhanging nucleosides.

Examples of single-stranded and double-stranded compounds include but are not limited to oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, a compound described herein has a nucleobase sequence that, when written in the 5′ to 3′ direction, comprises the reverse complement of the target segment of a target nucleic acid to which it is targeted.

In certain embodiments, a compound described herein comprises an oligonucleotide 10 to 30 linked subunits in length. In certain embodiments, compound described herein comprises an oligonucleotide is 12 to 30 linked subunits in length. In certain embodiments, compound described herein comprises an oligonucleotide is 12 to 22 linked subunits in length. In certain embodiments, compound described herein comprises an oligonucleotide is 14 to 30 linked subunits in length. In certain embodiments, compound described herein comprises an oligonucleotide is 14 to 20 linked subunits in length. In certain embodiments, compound described herein comprises an oligonucleotide is 15 to 30 linked subunits in length. In certain embodiments, compound described herein comprises an oligonucleotide is 15 to 20 linked subunits in length. In certain embodiments, compound described herein comprises an oligonucleotide is 16 to 30 linked subunits in length. In certain embodiments, compound described herein comprises an oligonucleotide is 16 to 20 linked subunits in length. In certain embodiments, compound described herein comprises an oligonucleotide is 17 to 30 linked subunits in length. In certain embodiments, compound described herein comprises an oligonucleotide is 17 to 20 linked subunits in length. In certain embodiments, compound described herein comprises an oligonucleotide is 18 to 30 linked subunits in length. In certain embodiments, compound described herein comprises an oligonucleotide is 18 to 21 linked subunits in length. In certain embodiments, compound described herein comprises an oligonucleotide is 18 to 20 linked subunits in length. In certain embodiments, compound described herein comprises an oligonucleotide is 20 to 30 linked subunits in length. In other words, such oligonucleotides are from 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits, respectively. In certain embodiments, a compound described herein comprises an oligonucleotide 14 linked subunits in length. In certain embodiments, a compound described herein comprises an oligonucleotide 16 linked subunits in length.

In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In other embodiments, a compound described herein comprises an oligonucleotide 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the linked subunits are nucleotides, nucleosides, or nucleobases.

In certain embodiments, compounds may be shortened or truncated. For example, a single subunit may be deleted from the 5′ end (5′ truncation), or alternatively from the 3′ end (3′ truncation). A shortened or truncated compound targeted to a HSD17B13 nucleic acid may have two subunits deleted from the 5′ end, or alternatively may have two subunits deleted from the 3′ end, of the compound. Alternatively, the deleted nucleosides may be dispersed throughout the compound.

When a single additional subunit is present in a lengthened compound, the additional subunit may be located at the 5′ or 3′ end of the compound. When two or more additional subunits are present, the added subunits may be adjacent to each other, for example, in a compound having two subunits added to the 5′ end (5′ addition), or alternatively to the 3′ end (3′ addition), of the compound. Alternatively, the added subunits may be dispersed throughout the compound.

It is possible to increase or decrease the length of a compound, such as an oligonucleotide, and/or introduce mismatch bases without eliminating activity (Woolf et al. (Proc. Natl. Acad. Sci. USA 89:7305-7309, 1992; Gautschi et al. J. Natl. Cancer Inst. 93:463-471, March 2001; Maher and Dolnick Nuc. Acid. Res. 16:3341-3358, 1988). However, seemingly small changes in oligonucleotide sequence, chemistry and motif can make large differences in one or more of the many properties required for clinical development (Seth et al. J. Med. Chem. 2009, 52, 10; Egli et al. J. Am. Chem. Soc. 2011, 133, 16642).

In certain embodiments, compounds described herein are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics.

In certain embodiments, a double-stranded compound comprises a first strand comprising the nucleobase sequence complementary to a target region of a HSD17B13 nucleic acid and a second strand. In certain embodiments, the double-stranded compound comprises ribonucleotides in which the first strand has uracil (U) in place of thymine (T) and is complementary to a target region. In certain embodiments, a double-stranded compound comprises (i) a first strand comprising a nucleobase sequence complementary to a target region of a HSD17B13 nucleic acid, and (ii) a second strand. In certain embodiments, the double-stranded compound comprises one or more modified nucleotides in which the 2′ position in the sugar contains a halogen (such as fluorine group; 2′-F) or contains an alkoxy group (such as a methoxy group; 2′-OMe). In certain embodiments, the double-stranded compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the double-stranded compound comprises one or more linkages between adjacent nucleotides other than a naturally-occurring phosphodiester linkage. Examples of such linkages include phosphoramide, phosphorothioate, and phosphorodithioate linkages. The double-stranded compounds may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000. In certain embodiments, the first strand of the double-stranded compound is an siRNA guide strand and the second strand of the double-stranded compound is an siRNA passenger strand. In certain embodiments, the second strand of the double-stranded compound is complementary to the first strand. In certain embodiments, each strand of the double-stranded compound consists of 16, 17, 18, 19, 20, 21, 22, or 23 linked nucleosides.

In certain embodiments, a single-stranded compound described herein can comprise any of the oligonucleotide sequences targeted to HSD17B13 described herein. In certain embodiments, such a single-stranded compound is a single-stranded RNAi (ssRNAi) compound. In certain embodiments, a ssRNAi compound comprises the nucleobase sequence complementary to a target region of a HSD17B13 nucleic acid. In certain embodiments, the ssRNAi compound comprises ribonucleotides in which uracil (U) is in place of thymine (T). In certain embodiments, ssRNAi compound comprises a nucleobase sequence complementary to a target region of a HSD17B13 nucleic acid. In certain embodiments, a ssRNAi compound comprises one or more modified nucleotides in which the 2′ position in the sugar contains a halogen (such as fluorine group; 2′-F) or contains an alkoxy group (such as a methoxy group; 2′-OMe). In certain embodiments, a ssRNAi compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the ssRNAi compound. In certain embodiments, the ssRNAi compound comprises one or more linkages between adjacent nucleotides other than a naturally-occurring phosphodiester linkage. Examples of such linkages include phosphoramide, phosphorothioate, and phosphorodithioate linkages. The ssRNAi compounds may also be chemically modified nucleic acid molecules as taught in U.S. Pat. No. 6,673,661. In other embodiments, the ssRNAi contains a capped strand, as disclosed, for example, by WO 00/63364, filed Apr. 19, 2000. In certain embodiments, the ssRNAi compound consists of 16, 17, 18, 19, 20, 21, 22, or 23 linked nucleosides.

In certain embodiments, compounds described herein comprise modified oligonucleotides. Certain modified oligonucleotides have one or more asymmetric center and thus give rise to enantiomers, diastereomers, and other stereoisomeric configurations that may be defined, in terms of absolute stereochemistry, as (R) or (S), as a or 13 such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

### Certain Mechanisms

In certain embodiments, compounds described herein comprise or consist of modified oligonucleotides. In certain embodiments, compounds described herein are antisense compounds. In certain embodiments, such antisense compounds comprise oligomeric compounds. In certain embodiments, compounds described herein are capable of hybridizing to a target nucleic acid, resulting in at least one antisense activity. In certain embodiments, compounds described herein selectively affect one or more target nucleic acid. Such selective compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in the gap of a gapmer is tolerated.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain embodiments, hybridization of compounds described herein to a target nucleic acid does not result in recruitment of a protein that cleaves that target nucleic acid. In certain such embodiments, hybridization of the compound to the target nucleic acid results in alteration of splicing of the target nucleic acid. In certain embodiments, hybridization of the compound to a target nucleic acid results in inhibition of a binding interaction between the target nucleic acid and a protein or other nucleic acid. In certain such embodiments, hybridization of the compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or individual.

### Target Nucleic Acids, Target Regions and Nucleotide Sequences

In certain embodiments, compounds described herein comprise or consist of an oligonucleotide comprising a region that is complementary to a target nucleic acid. In certain embodiments, the target nucleic acid is an endogenous RNA molecule. In certain such embodiments, the target nucleic acid is selected from: an mRNA and a pre-mRNA, including intronic, exonic and untranslated regions. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

Nucleotide sequences that encode HSD17B13 include, without limitation, the following: RefSEQ Nos. NM_001163486.1 (incorporated by reference, disclosed herein as SEQ ID NO: 1); NM_198030.2 (incorporated by reference, disclosed herein as SEQ ID NO: 2); NC_000071.6_TRUNC_103952001_103980000_COMP (incorporated by reference, disclosed herein as SEQ ID NO: 3); NM_001136230.2 (incorporated by reference, disclosed herein as SEQ ID NO: 4); NM_178135.4 (incorporated by reference, disclosed herein as SEQ ID NO: 5); and NC_000004.12_TRUNC_87301001_87326000_COMP (incorporated by reference, disclosed herein as SEQ ID NO: 6).

### Hybridization

In some embodiments, hybridization occurs between a compound disclosed herein and a HSD17B13 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the compounds provided herein are specifically hybridizable with a HSD17B13 nucleic acid.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to adenine (A) and thymine (T), adenine (A) and uracil (U), cytosine (C) and guanine (G), and 5-methyl cytosine (mC) and guanine (G) unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

In certain embodiments, compounds described herein comprise or consist of modified oligonucleotides. In certain embodiments, compounds described herein are antisense compounds. In certain embodiments, compounds comprise oligomeric compounds. Non-complementary nucleobases between a compound and a HSD17B13 nucleic acid may be tolerated provided that the compound remains able to specifically hybridize to a target nucleic acid. Moreover, a compound may hybridize over one or more segments of a HSD17B13 nucleic acid such that intervening or adjacent segments are not involved in the hybridization event (e.g., a loop structure, mismatch or hairpin structure).

In certain embodiments, the compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a HSD17B13 nucleic acid, a target region, target segment, or specified portion thereof. Percent complementarity of a compound with a target nucleic acid can be determined using routine methods.

For example, a compound in which 18 of 20 nucleobases of the compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, a compound which is 18 nucleobases in length having four non-complementary nucleobases which are flanked by two regions of complete complementarity with the target nucleic acid would have 77.8% overall complementarity with the target nucleic acid and would thus fall within the scope of the present invention. Percent complementarity of a compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, compounds described herein, or specified portions thereof, are fully complementary (i.e. 100% complementary) to a target nucleic acid, or specified portion thereof. For example, a compound may be fully complementary to a HSD17B13 nucleic acid, or a target region, or a target segment or target sequence thereof. As used herein, “fully complementary” means each nucleobase of a compound is capable of precise base pairing with the corresponding nucleobases of a target nucleic acid. For example, a 20 nucleobase compound is fully complementary to a target sequence that is 400 nucleobases long, so long as there is a corresponding 20 nucleobase portion of the target nucleic acid that is fully complementary to the compound. Fully complementary can also be used in reference to a specified portion of the first and/or the second nucleic acid. For example, a 20 nucleobase portion of a 30 nucleobase compound can be “fully complementary” to a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence.

In certain embodiments, compounds described herein comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position, 2, 3, 4, 5, 6, 7, 8, 9, 10, 11, or 12 from the 3′-end of the oligonucleotide.

The location of a non-complementary nucleobase may be at the 5′ end or 3′ end of the compound. Alternatively, the non-complementary nucleobase or nucleobases may be at an internal position of the compound. When two or more non-complementary nucleobases are present, they may be contiguous (i.e. linked) or non-contiguous. In one embodiment, a non-complementary nucleobase is located in the wing segment of a gapmer oligonucleotide.

In certain embodiments, compounds described herein that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a HSD17B13 nucleic acid, or specified portion thereof.

In certain embodiments, compounds described herein that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a HSD17B13 nucleic acid, or specified portion thereof.

In certain embodiments, compounds described herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of a compound. In certain embodiments, the compounds are complementary to at least an 8 nucleobase portion of a target segment. In certain embodiments, the compounds are complementary to at least a 9 nucleobase portion of a target segment. In certain embodiments, the compounds are complementary to at least a 10 nucleobase portion of a target segment. In certain embodiments, the compounds are complementary to at least an 11 nucleobase portion of a target segment. In certain embodiments, the compounds are complementary to at least a 12 nucleobase portion of a target segment. In certain embodiments, the compounds are complementary to at least a 13 nucleobase portion of a target segment.

In certain embodiments, the compounds are complementary to at least a 14 nucleobase portion of a target segment. In certain embodiments, the compounds are complementary to at least a 15 nucleobase portion of a target segment. In certain embodiments, the compounds are complementary to at least a 16 nucleobase portion of a target segment. Also contemplated are compounds that are complementary to at least a 9, 10, 11, 12, 13, 14, 15, 16, 17, 18, 19, 20, or more nucleobase portion of a target segment, or a range defined by any two of these values.

### Identity

The compounds provided herein may also have a defined percent identity to a particular nucleotide sequence, SEQ ID NO, or compound represented by a specific Isis number, or portion thereof. In certain embodiments, compounds described herein are antisense compounds or oligomeric compounds. In certain embodiments, compounds described herein are modified oligonucleotides. As used herein, a compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the compounds described herein as well as compounds having non-identical bases relative to the compounds provided herein also are contemplated. The non-identical bases may be adjacent to each other or dispersed throughout the compound. Percent identity of a compound is calculated according to the number of bases that have identical base pairing relative to the sequence to which it is being compared.

In certain embodiments, compounds described herein, or portions thereof, are, or are at least, 70%, 75%, 80%, 85%, 90%, 91%, 92%, 93%, 94%, 95%, 96%, 97%, 98%, 99% or 100% identical to one or more of the compounds or SEQ ID NOs, or a portion thereof, disclosed herein. In certain embodiments, compounds described herein are about 70%, 75%, 80%, 85%, 90%, 91%, 92%, 93%, 94%, 95%, 96%, 97%, 98%, or 99% identical, or any percentage between such values, to a particular nucleotide sequence, SEQ ID NO, or compound represented by a specific Isis number, or portion thereof, in which the compounds comprise an oligonucleotide having one or more mismatched nucleobases. In certain such embodiments, the mismatch is at position 1, 2, 3, 4, 5, 6, 7, 8, 9, 10, 11, or 12 from the 5′-end of the oligonucleotide. In certain such embodiments, the mismatch is at position 2, 3, 4, 5, 6, 7, 8, 9, 10, 11, or 12 from the 3′-end of the oligonucleotide.

In certain embodiments, compounds described herein are antisense compounds. In certain embodiments, a portion of the compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, compounds described herein are oligonucleotides. In certain embodiments, a portion of the oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Certain Modified Compounds

In certain embodiments, compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

A. Modified Nucleosides

Modified nucleosides comprise a modified sugar moiety or a modified nucleobase or both a modified sugar moiety and a modified nucleobase.

1. Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for linearlynon-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or 2′-non-bicyclic modified nucleoside comprises a sugar moiety comprising a linear 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a linear 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a linear 2′-substituent group selected from: F, OCH₃, and OCH₂CH₂OCH₃.

Nucleosides comprising modified sugar moieties, such as non-bicyclic modified sugar moieties, are referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₙ)(R_{b})]ₙ—, —[C(Rₙ)(R_{b})]ₙ—O—, —C(Rₙ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U S. A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 91999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

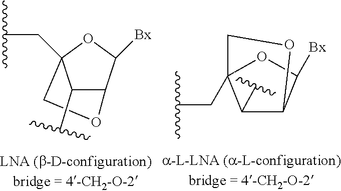

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see e.g., Leumann, C J. Bioorg. & Med. Chem. 2002, 10, 841-854), fluoro HNA:

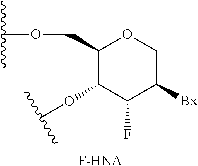

(“F-HNA”, see e.g., Swayze et al., U.S. Pat. No. 8,088,904; Swayze et al., U.S. Pat. No. 8,440,803; Swayze et al., U.S.; and Swayze et al., U.S. Pat. No. 9,005,906, F-HNA can also be referred to as a F-THP or 3′-fluoro tetrahydropyran), and nucleosides comprising additional modified THP compounds having the formula:

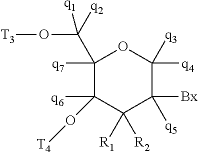

wherein, independently, for each of said modified THP nucleoside: Bx is a nucleobase moiety; T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

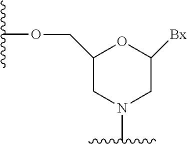

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to compounds described herein.

In certain embodiments, compounds described herein comprise modified oligonucleotides. In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimi¬dines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C═C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds targeted to a HSD17B13 nucleic acid comprise one or more modified nucleobases. In certain embodiments, the modified nucleobase is 5-methylcytosine. In certain embodiments, each cytosine is a 5-methylcytosine.

### Modified Internucleoside Linkages

The naturally occurring internucleoside linkage of RNA and DNA is a 3′ to 5′ phosphodiester linkage. In certain embodiments, compounds described herein having one or more modified, i.e. non-naturally occurring, internucleoside linkages are often selected over compounds having naturally occurring internucleoside linkages because of desirable properties such as, for example, enhanced cellular uptake, enhanced affinity for target nucleic acids, and increased stability in the presence of nucleases.

In certain embodiments, compounds targeted to a HSD17B13 nucleic acid comprise one or more modified internucleoside linkages. In certain embodiments, the modified internucleoside linkages are phosphorothioate linkages. In certain embodiments, each internucleoside linkage of the compound is a phosphorothioate internucleoside linkage.

In certain embodiments, compounds described herein comprise oligonucleotides. Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH2-O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and if it does have a gapped nucleoside motif, the wing and gap lengths may or may not be the same.

In certain embodiments, oligonucleotides comprise a region having an alternating internucleoside linkage motif. In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosponate linkages. In certain embodiments, oligonucleotides having a gapmer nucleoside motif comprise a linkage motif comprising all phosphorothioate linkages except for one or two methylphosponate linkages. In certain embodiments, one methylphosponate linkage is in the central gap of an oligonucleotide having a gapmer nucleoside motif.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

B. Certain Motifs

In certain embodiments, compounds described herein comprise oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, compounds described herein comprise oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, a modified oligonucleotide has a fully modified sugar motif wherein each nucleoside of the modified oligonucleotide comprises a modified sugar moiety. In certain embodiments, modified oligonucleotides comprise or consist of a region having a fully modified sugar motif wherein each nucleoside of the region comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

2. Certain Nucleobase Motifs

In certain embodiments, compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, compounds described herein comprise oligonucleotides. In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

C. Certain Modified Oligonucleotides

In certain embodiments, compounds described herein comprise modified oligonucleotides. In certain embodiments, the above modifications (sugar, nucleobase, internucleoside linkage) are incorporated into a modified oligonucleotide. In certain embodiments, modified oligonucleotides are characterized by their modification, motifs, and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., a regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

### Certain Conjugated Compounds

In certain embodiments, the compounds described herein comprise or consist of an oligonucleotide (modified or unmodified) and, optionally, one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2¹-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

In certain embodiments, the oligonucleotide is modified. In certain embodiments, the oligonucleotide of a compound has a nucleobase sequence that is complementary to a target nucleic acid. In certain embodiments, oligonucleotides are complementary to a messenger RNA (mRNA). In certain embodiments, oligonucleotides are complementary to a pre-mRNA. In certain embodiments, oligonucleotides are complementary to a sense transcript.

Examples of terminal groups include but are not limited to conjugate groups, capping groups, phosphate moieties, protecting groups, modified or unmodified nucleosides, and two or more nucleosides that are independently modified or unmodified.

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain compounds, the conjugate linker is a single chemical bond (i.e., the conjugate moiety is attached directly to an oligonucleotide through a single bond). In certain compounds, a conjugate moiety is attached to an oligonucleotide via a more complex conjugate linker comprising one or more conjugate linker moeities, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which an compound comprises an oligonucleotide consisting of a specified number or range of linked nucleosides and/or a specified percent complementarity to a reference nucleic acid and the compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, a compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, a compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances compounds comprising a particular conjugate moiety are better taken up by a particular cell type, but once the compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

3. Certain Cell-Targeting Conjugate Moieties

In certain embodiments, a conjugate group comprises a cell-targeting conjugate moiety. In certain embodiments, a conjugate group has the general formula:

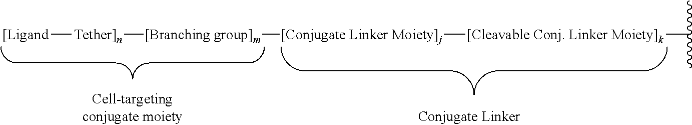

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactoseamine (GalNAc), mannose, glucose, glucoseamine and fucose. In certain embodiments, each ligand is N-acetyl galactoseamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29 or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, conjugate groups comprise a cell-targeting moiety having the formula:

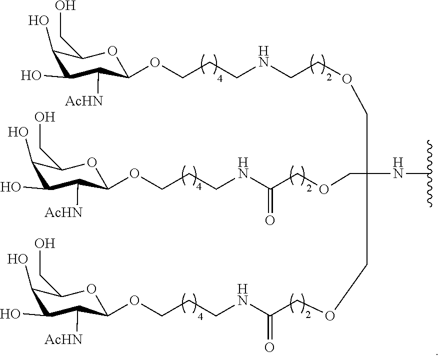

In certain embodiments, conjugate groups comprise a cell-targeting moiety having the formula:

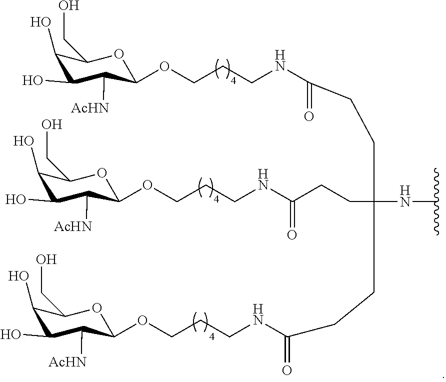

In certain embodiments, conjugate groups comprise a cell-targeting moiety having the formula:

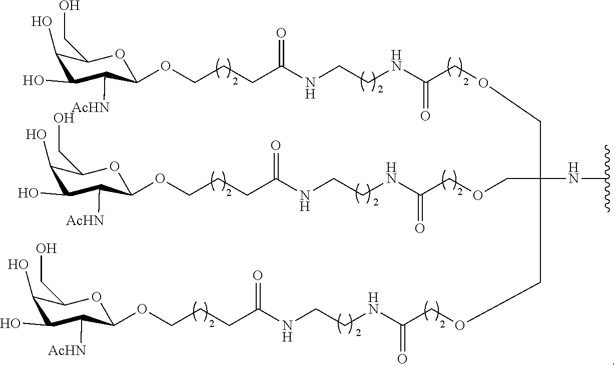

In certain embodiments, conjugate groups comprise a cell-targeting moiety having the formula:

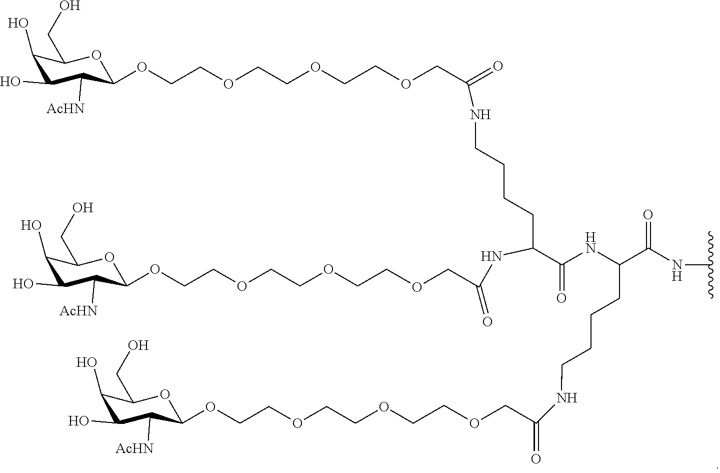

In certain embodiments, conjugate groups comprise a cell-targeting moiety having the formula:

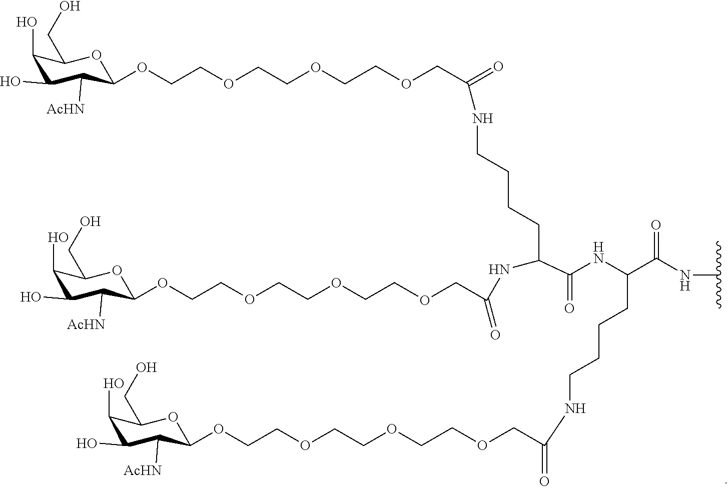

In certain embodiments, compounds comprise a conjugate group described herein as “LICA-1”. LICA-1 has the formula:

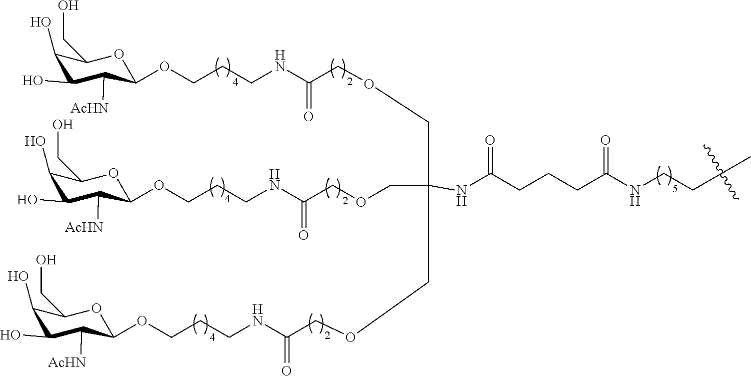

In certain embodiments, compounds described herein comprise LICA-1 and a cleavable moiety within the conjugate linker have the formula:

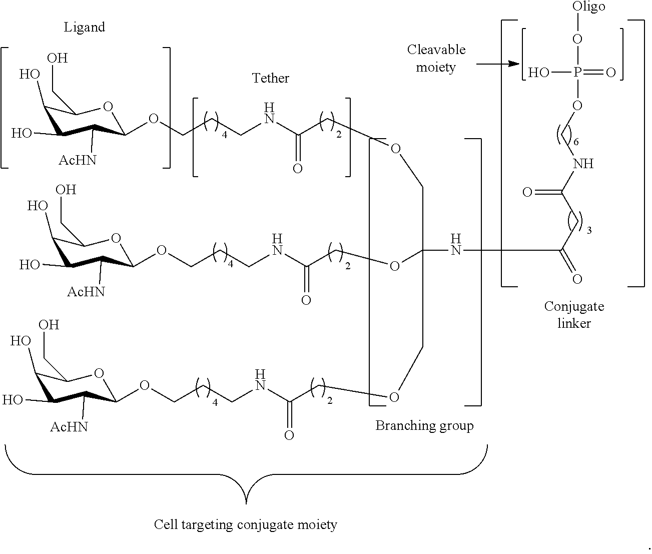

wherein oligo is an oligonucleotide.

Representative United States patents, United States patent application publications, international patent application publications, and other publications that teach the preparation of certain of the above noted conjugate groups, compounds comprising conjugate groups, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, Biessen et al., J Med. Chem. 1995, 38, 1846-1852, Lee et al., Bioorganic & Medicinal Chemistry 2011, 19, 2494-2500, Rensen et al., Biol. Chem. 2001, 276, 37577-37584, Rensen et al., J Med. Chem. 2004, 47, 5798-5808, Sliedregt et al., Med. Chem. 1999, 42, 609-618, and Valentijn et al., Tetrahedron, 1997, 53, 759-770.

In certain embodiments, modified oligonucleotides comprise a gapmer or fully modified sugar motif and a conjugate group comprising at least one, two, or three GalNAc ligands. In certain embodiments, compounds comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

In certain embodiments, compounds are single-stranded. In certain embodiments, compounds are double-stranded.

### Compositions and Methods for Formulating Pharmaceutical Compositions

Compounds described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

In certain embodiments, the present invention provides pharmaceutical compositions comprising one or more compounds or a salt thereof. In certain embodiments, the compounds are antisense compounds or oligomeric compounds. In certain embodiments, the compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more compound. In certain embodiments, such pharmaceutical composition consists of a sterile saline solution and one or more compound. In certain embodiments, the sterile saline is pharmaceutical grade saline. In certain embodiments, a pharmaceutical composition comprises one or more compound and sterile water. In certain embodiments, a pharmaceutical composition consists of one compound and sterile water. In certain embodiments, the sterile water is pharmaceutical grade water. In certain embodiments, a pharmaceutical composition comprises one or more compound and phosphate-buffered saline (PBS). In certain embodiments, a pharmaceutical composition consists of one or more compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

A compound described herein targeted to a HSD17B13 nucleic acid can be utilized in pharmaceutical compositions by combining the compound with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising a compound targeted to a HSD17B13 nucleic acid and a pharmaceutically acceptable diluent. In certain embodiments, the pharmaceutically acceptable diluent is water. In certain embodiments, the compound comprises or consists of a modified oligonucleotide provided herein.

Pharmaceutical compositions comprising compounds provided herein encompass any pharmaceutically acceptable salts, esters, or salts of such esters, or any other oligonucleotide which, upon administration to an individual, including a human, is capable of providing (directly or indirectly) the biologically active metabolite or residue thereof. In certain embodiments, the compounds are antisense compounds or oligomeric compounds. In certain embodiments, the compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of a compound which are cleaved by endogenous nucleases within the body, to form the active compound.

In certain embodiments, the compounds or compositions further comprise a pharmaceutically acceptable carrier or diluent.

### Certain Combinations and Combination Therapies

In certain embodiments, a first agent comprising the compound described herein is co-administered with one or more secondary agents. In certain embodiments, such second agents are designed to treat the same disease, disorder, or condition as the first agent described herein. In certain embodiments, such second agents are designed to treat a different disease, disorder, or condition as the first agent described herein. In certain embodiments, a first agent is designed to treat an undesired side effect of a second agent. In certain embodiments, second agents are co-administered with the first agent to treat an undesired effect of the first agent. In certain embodiments, such second agents are designed to treat an undesired side effect of one or more pharmaceutical compositions as described herein. In certain embodiments, second agents are co-administered with the first agent to produce a combinational effect. In certain embodiments, second agents are co-administered with the first agent to produce a synergistic effect. In certain embodiments, the co-administration of the first and second agents permits use of lower dosages than would be required to achieve a therapeutic or prophylactic effect if the agents were administered as independent therapy.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Effect of 3-10-3 cEt Gapmers with Phosphorothioate Internucleoside Linkages on HSD17B13 In Vitro, Single Dose

Modified oligonucleotides complementary to a HSD17B13 nucleic acid were designed and tested for their effect on HSD17B13 mRNA in vitro.

Mouse primary hepatocyte cells at a density of 20,000 cells per well were transfected by free uptake with 2,000 nM concentration of modified oligonucleotide or no modified oligonucleotide for untreated controls. After approximately 24 hours, RNA was isolated from the cells and HSD17B13 mRNA levels were measured by quantitative real-time PCR. Mouse primer probe set RTS40764 (forward sequence AATAAGCGTGGTGTTGAGGAA, designated herein as SEQ ID NO: 7; reverse sequence CGACATCACCTACTTCTCTCTT, designated herein as SEQ ID NO: 8; probe sequence TTGTAAATCTCGGCCCGGTTGCT, designated herein as SEQ ID: 9) was used to measure mRNA levels. HSD17B13 mRNA levels were adjusted according to total RNA content, as measured by RIBOGREEN®. Results are presented in the table below as percent control of the amount of HSD17B13 mRNA, relative to untreated control cells. The modified oligonucleotides with percent control values marked with an asterisk (*) target the amplicon region of the primer probe set. Additional assays may be used to measure the potency and efficacy of oligonucleotides targeting the amplicon region.

The modified oligonucleotides on Table 1 are 3-10-3 cEt gapmers. The gapmers are 16 nucleobases in length, wherein the central gap segment comprises ten 2′-deoxynucleosides and is flanked by wing segments on both the 5′ end and on the 3′ end comprising three cEt nucleosides. The sugar motif for the gapmers is (from 5′ to 3′): kkkddddddddddkkk; wherein ‘d’ represents a 2′-deoxyribose sugar and ‘k’ represents a cEt modified sugar. Each internucleoside linkage is a phosphorothioate internucleoside linkage and each cytosine residue is a 5′-methyl cytosine. “Start Site” indicates the 5′-most nucleoside to which the gapmer is complementary in the mouse nucleic acid sequence. “Stop Site” indicates the 3′-most nucleoside to which the gapmer is complementary in the mouse nucleic acid sequence.

Each modified oligonucleotide listed in Tables 1 through 4 below is complementary to mouse HSD17B13 nucleic acid sequences GENBANK Accession No. NM_198030.2 (SEQ ID NO: 2) or SEQ ID NO: 3 (the complement of NC_000071.6 truncated from nucleotides 103952001 to 103980000), as indicated. ‘N/A’ indicates that the modified oligonucleotide is not complementary to that particular nucleic acid sequence with 100% complementarity. As shown below, modified oligonucleotides complementary to HSD17B13 reduced the amount of HSD17B13 mRNA. N.d. indicates that there was no data for that particular oligonucleotide.

TABLE 1
Percent control of HSD17B13 mRNA with 3-10-3 cEt gapmers
with phosphorothioate internucleoside linkages

| | SEQ | SEQ | SEQ | SEQ | | | |
|---|---|---|---|---|---|---|---|
| | ID No: | ID No: | ID No: | ID No: | | HSD17B13 | SEQ |
| Compound | 1 Start | 1 Stop | 2 Start | 2 Stop | | (% | ID |
| Number | Site | Site | Site | Site | Sequence (5′ to 3′) | control) | NO |
| 1145895 | 4 | 19 | 2616 | 2631 | AGGTATTATGGGCTGC | 73 | 10 |
| 1145903 | 177 | 192 | 2789 | 2804 | AGAACGGTCTGCCCGG | 28 | 11 |
| 1145923 | 363 | 378 | 7406 | 7421 | TTGTAAATCTCGGCCC | n.d. | 12 |
| 1145927 | 409 | 424 | 9125 | 9140 | CACGATCTCGACATCA | n.d. | 13 |
| 1145939 | 452 | 467 | 9168 | 9183 | CACTAAGAAGGTCTGC | 43 | 14 |
| 1145963 | 679 | 694 | 12941 | 12956 | CCCCAAGGTGTCCAGT | 27 | 15 |
| 1146007 | 1063 | 1078 | 23892 | 23907 | CAGTAATAGTAGCACA | 74 | 16 |
| 1146015 | 1159 | 1174 | 23988 | 24003 | TTTATACAGTCAGAGT | 82 | 17 |
| 1146019 | 1198 | 1213 | 24027 | 24042 | ATATTTTGGCAGAAGG | 83 | 18 |
| 1146023 | 1376 | 1391 | 24205 | 24220 | TCCCATTACATGGGTT | 86 | 19 |
| 1146027 | 1431 | 1446 | 24260 | 24275 | TACCAAGGCATGGGTA | 72 | 20 |
| 1146035 | N/A | N/A | 2901 | 2916 | GTTAGAAACACCTATT | 36 | 21 |
| 1146039 | N/A | N/A | 3518 | 3533 | GAGAATCAATCCCTCA | 40 | 22 |
| 1146043 | N/A | N/A | 3672 | 3687 | TATTATTCTTTACCCT | 36 | 23 |
| 1146047 | N/A | N/A | 3953 | 3968 | TAATTGTTGTACCGCT | 42 | 24 |
| 1146051 | N/A | N/A | 4049 | 4064 | TCCGGTACATGACAGC | 46 | 25 |
| 1146055 | N/A | N/A | 4452 | 4467 | TATTTTTTACGAGGGA | 51 | 26 |
| 1146059 | N/A | N/A | 4540 | 4555 | AAGTATTGATGTCTTC | 40 | 27 |
| 1146063 | N/A | N/A | 5526 | 5541 | ATAATTAATCTGGAGC | 39 | 28 |
| 1146067 | N/A | N/A | 6015 | 6030 | CCACTAATGTTGGCTT | 28 | 29 |
| 1146071 | N/A | N/A | 6653 | 6668 | CTTACCTAAGATTGTC | 54 | 30 |
| 1146075 | N/A | N/A | 7036 | 7051 | AGTTATCGAAGATGCT | 37 | 31 |
| 1146087 | N/A | N/A | 8861 | 8876 | AGCATAAACTAGGCCA | 53 | 32 |
| 1146099 | N/A | N/A | 10086 | 10101 | AGAACTAATAGGCATG | 39 | 33 |
| 1146103 | N/A | N/A | 10191 | 10206 | CGGTATTAATTCATAC | 50 | 34 |
| 1146107 | N/A | N/A | 10216 | 10231 | GTGCTATAGTAATTTT | 42 | 35 |
| 1146111 | N/A | N/A | 10457 | 10472 | TAAATTCCTAGAGCCC | 34 | 36 |
| 1146119 | N/A | N/A | 11148 | 11163 | ACACGGTTATTAGGTG | 83 | 37 |
| 1146123 | N/A | N/A | 11459 | 11474 | CCTATAATTAATCCCT | 52 | 38 |
| 1146127 | N/A | N/A | 12121 | 12136 | GCATATATGGAGCTAT | 31 | 39 |
| 1146135 | N/A | N/A | 13093 | 13108 | CTTGTTAAGTACCTAT | 42 | 40 |
| 1146139 | N/A | N/A | 13495 | 13510 | CGTGTATAACTGAGAA | 62 | 41 |
| 1146143 | N/A | N/A | 14234 | 14249 | TCAGGGTTCTGCGAGG | 68 | 42 |
| 1146147 | N/A | N/A | 15287 | 15302 | CGTAACAAATCACCCA | 49 | 43 |
| 1146151 | N/A | N/A | 15712 | 15727 | AGCGGTTAAACATGGC | 63 | 44 |
| 1146155 | N/A | N/A | 16032 | 16047 | CTTACTCAAGTCCAGT | 47 | 45 |
| 1146159 | N/A | N/A | 16627 | 16642 | ATTCATATGTCAAGGA | 67 | 46 |
| 1146163 | N/A | N/A | 17497 | 17512 | GATACTTATATTCAGC | 48 | 47 |
| 1146167 | N/A | N/A | 18219 | 18234 | TATATTAGATGACAGA | 106 | 48 |
| 1146175 | N/A | N/A | 18645 | 18660 | AAGGATAGTTTAATCT | 91 | 49 |
| 1146179 | N/A | N/A | 19770 | 19785 | AATAGGTGAAGGAGTT | 101 | 50 |
| 1146183 | N/A | N/A | 21222 | 21237 | AGTGAACACACCTAGC | 50 | 51 |
| 1146191 | N/A | N/A | 22396 | 22411 | GATAGGTTGATCAGGA | 106 | 52 |
| 1146195 | N/A | N/A | 22454 | 22469 | GAGCATATATTAATGG | 130 | 53 |
| 1146199 | N/A | N/A | 22612 | 22627 | CAGATTAATGCTAGAG | 77 | 54 |

TABLE 2
Percent control of HSD17B13 mRNA with 3-10-3 cEt gapmers
with phosphorothioate internucleoside linkages

| | SEQ | SEQ | SEQ | SEQ | | | |
|---|---|---|---|---|---|---|---|
| | ID No: | ID No: | ID No: | ID No: | | HSD17B13 | SEQ |
| Compound | 1 Start | 1 Stop | 2 Start | 2 Stop | | (% | ID |
| Number | Site | Site | Site | Site | Sequence (5′ to 3′) | control) | NO |
| 1145896 | 5 | 20 | 2617 | 2632 | CAGGTATTATGGGCTG | 106 | 55 |
| 1145900 | 133 | 148 | 2745 | 2760 | GAACTTTACCAGTGAC | 18 | 56 |
| 1145916 | 291 | 306 | 7334 | 7349 | TCCGCGGTTTCCTCAA | n.d. | 57 |
| 1145920 | 357 | 372 | 7400 | 7415 | ATCTCGGCCCGGTTGC | n.d. | 58 |
| 1145924 | 369 | 384 | 7412 | 7427 | ACAGAGTTGTAAATCT | n.d. | 59 |
| 1145928 | 410 | 425 | 9126 | 9141 | CCACGATCTCGACATC | n.d. | 60 |
| 1145932 | 435 | 450 | 9151 | 9166 | GGATATATCGCCCCGG | 36 | 61 |
| 1145940 | 453 | 468 | 9169 | 9184 | GCACTAAGAAGGTCTG | 26 | 62 |
| 1146004 | 1017 | 1032 | 23846 | 23861 | GTATGAGGGCTTGCCT | 71 | 63 |
| 1146008 | 1064 | 1079 | 23893 | 23908 | TCAGTAATAGTAGCAC | 90 | 64 |
| 1146012 | 1126 | 1141 | 23955 | 23970 | GACATTATCTACAATA | 94 | 65 |
| 1146016 | 1161 | 1176 | 23990 | 24005 | GGTTTATACAGTCAGA | 49 | 66 |
| 1146020 | 1212 | 1227 | 24041 | 24056 | CTTAAGTGTTGTAAAT | 56 | 67 |
| 1146024 | 1393 | 1408 | 24222 | 24237 | CTGAATCCCATCTGTC | 104 | 68 |
| 1146028 | 1433 | 1448 | 24262 | 24277 | TATACCAAGGCATGGG | 114 | 69 |
| 1146032 | 1438 | 1453 | 24267 | 24282 | ACTCATATACCAAGGC | 99 | 70 |
| 1146036 | N/A | N/A | 3204 | 3219 | GTAAGTTATGTGGCTT | 30 | 71 |
| 1146040 | N/A | N/A | 3600 | 3615 | TATTTTAGGATTGCTG | 35 | 72 |
| 1146044 | N/A | N/A | 3673 | 3688 | GTATTATTCTTTACCC | 36 | 73 |
| 1146048 | N/A | N/A | 3955 | 3970 | GATAATTGTTGTACCG | 33 | 74 |
| 1146056 | N/A | N/A | 4453 | 4468 | ATATTTTTTACGAGGG | 48 | 75 |
| 1146060 | N/A | N/A | 4612 | 4627 | ATCTTTAATGTGACCT | 46 | 76 |
| 1146064 | N/A | N/A | 5592 | 5607 | CTTACGGTGAAACCTA | 50 | 77 |
| 1146068 | N/A | N/A | 6148 | 6163 | GACTTTAAGGAGGGTT | 44 | 78 |
| 1146072 | N/A | N/A | 6682 | 6697 | GCTAATTTTTAGCCTA | 45 | 79 |
| 1146076 | N/A | N/A | 7043 | 7058 | TCCTATAAGTTATCGA | 41 | 80 |
| 1146080 | N/A | N/A | 7655 | 7670 | CTTGATAAATCATCTT | 49 | 81 |
| 1146088 | N/A | N/A | 8862 | 8877 | CAGCATAAACTAGGCC | 50 | 82 |
| 1146092 | N/A | N/A | 9302 | 9317 | ACAAATATTGTGACCT | 47 | 83 |
| 1146096 | N/A | N/A | 9959 | 9974 | GTAACTCAATTGTGAA | 49 | 84 |
| 1146100 | N/A | N/A | 10088 | 10103 | CCAGAACTAATAGGCA | 40 | 85 |
| 1146104 | N/A | N/A | 10194 | 10209 | CCACGGTATTAATTCA | 60 | 86 |
| 1146108 | N/A | N/A | 10303 | 10318 | AGTATATAGGGTCCCT | 54 | 87 |
| 1146112 | N/A | N/A | 10623 | 10638 | CTCTATCCTGGCCCAC | 43 | 88 |
| 1146116 | N/A | N/A | 11140 | 11155 | ATTAGGTGGATTCCAG | 83 | 89 |
| 1146124 | N/A | N/A | 11460 | 11475 | TCCTATAATTAATCCC | 79 | 90 |
| 1146128 | N/A | N/A | 12123 | 12138 | ATGCATATATGGAGCT | 59 | 91 |
| 1146132 | N/A | N/A | 12377 | 12392 | ACATCGACAAACTTGT | 51 | 92 |
| 1146136 | N/A | N/A | 13245 | 13260 | CTTTTTAGATTATCCT | 69 | 93 |
| 1146140 | N/A | N/A | 13598 | 13613 | CGTACTAAGATTTGCT | 34 | 94 |
| 1146152 | N/A | N/A | 15713 | 15728 | CAGCGGTTAAACATGG | 56 | 95 |
| 1146156 | N/A | N/A | 16149 | 16164 | GCTTTTAAGGCACGCT | 26 | 96 |
| 1146160 | N/A | N/A | 16861 | 16876 | TATGTATACGGTTGGG | 64 | 97 |
| 1146164 | N/A | N/A | 17576 | 17591 | ATTATATGCTCCGGAA | 98 | 98 |
| 1146176 | N/A | N/A | 18661 | 18676 | GATTTTAGTGGCAGCC | 98 | 99 |
| 1146180 | N/A | N/A | 20113 | 20128 | AGTACTAACAATGCAG | 122 | 100 |
| 1146184 | N/A | N/A | 21667 | 21682 | TGATTTACCCAGTGGT | 82 | 101 |
| 1146188 | N/A | N/A | 21946 | 21961 | AAGGCATAATTCATTA | 87 | 102 |
| 1146192 | N/A | N/A | 22436 | 22451 | TGACTAAATATGCCTC | 102 | 103 |
| 1146196 | N/A | N/A | 22560 | 22575 | ATTTTTAACCTACGCA | 102 | 104 |
| 1146200 | N/A | N/A | 22613 | 22628 | CCAGATTAATGCTAGA | 113 | 105 |
| 1146204 | N/A | N/A | 23535 | 23550 | GGACTATTGATCTTCA | 130 | 106 |

TABLE 3
Percent control of HSD17B13 mRNA with 3-10-3 cEt gapmers
with phosphorothioate internucleoside linkages

| | SEQ | SEQ | SEQ | SEQ | | | |
|---|---|---|---|---|---|---|---|
| | ID No: | ID No: | ID No: | ID No: | | HSD17B13 | SEQ |
| Compound | 1 Start | 1 Stop | 2 Start | 2 Stop | | (% | ID |
| Number | Site | Site | Site | Site | Sequence (5′ to 3′) | control) | NO |
| 1145893 | 1 | 16 | 2613 | 2628 | TATTATGGGCTGCTGC | 69 | 107 |
| 1145901 | 135 | 150 | 2747 | 2762 | AAGAACTTTACCAGTG | 33 | 108 |
| 1145917 | 300 | 315 | 7343 | 7358 | CTGCATTTGTCCGCGG | n.d. | 109 |
| 1145921 | 360 | 375 | 7403 | 7418 | TAAATCTCGGCCCGGT | n.d. | 110 |
| 1145929 | 411 | 426 | 9127 | 9142 | ACCACGATCTCGACAT | n.d. | 111 |
| 1145933 | 436 | 451 | 9152 | 9167 | TGGATATATCGCCCCG | 28 | 112 |
| 1145941 | 455 | 470 | 9171 | 9186 | TGGCACTAAGAAGGTC | 40 | 113 |
| 1145961 | 651 | 666 | 12913 | 12928 | GCTCGGTGGAAGCCCA | 54 | 114 |
| 1145985 | 779 | 794 | 14120 | 14135 | CTTCCGGCTCTAATAC | 49 | 115 |
| 1146005 | 1060 | 1075 | 23889 | 23904 | TAATAGTAGCACATTT | 60 | 116 |
| 1146009 | 1065 | 1080 | 23894 | 23909 | ATCAGTAATAGTAGCA | 99 | 117 |
| 1146021 | 1230 | 1245 | 24059 | 24074 | AAAAATCAGCCCTTAT | 107 | 118 |
| 1146025 | 1418 | 1433 | 24247 | 24262 | GTACAATGATCAGAGG | 105 | 119 |
| 1146029 | 1434 | 1449 | 24263 | 24278 | ATATACCAAGGCATGG | 70 | 120 |
| 1146033 | 1441 | 1456 | 24270 | 24285 | GATACTCATATACCAA | 108 | 121 |
| 1146037 | N/A | N/A | 3265 | 3280 | AGATTTTATCCCAATG | 59 | 122 |
| 1146041 | N/A | N/A | 3601 | 3616 | GTATTTTAGGATTGCT | 47 | 123 |
| 1146045 | N/A | N/A | 3808 | 3823 | AGACTTAAGGTAGTTA | 49 | 124 |
| 1146049 | N/A | N/A | 3956 | 3971 | AGATAATTGTTGTACC | 49 | 125 |
| 1146053 | N/A | N/A | 4378 | 4393 | GATTTGATAATCTCAG | 52 | 126 |
| 1146057 | N/A | N/A | 4454 | 4469 | TATATTTTTTACGAGG | 49 | 127 |
| 1146061 | N/A | N/A | 4744 | 4759 | AGTTACACTTGCAGCT | 43 | 128 |
| 1146065 | N/A | N/A | 5647 | 5662 | AGAGATAATGATGGGT | 38 | 129 |
| 1146069 | N/A | N/A | 6269 | 6284 | TCATTTGGGCCTTGCC | 49 | 130 |
| 1146073 | N/A | N/A | 6797 | 6812 | AGTCTTAACTGAGTAT | 48 | 131 |
| 1146077 | N/A | N/A | 7134 | 7149 | TTCGGGTTAAGGCTTT | 49 | 132 |
| 1146081 | N/A | N/A | 7753 | 7768 | ATTATACGCAAACCAA | 37 | 133 |
| 1146085 | N/A | N/A | 8732 | 8747 | GATATCGATCTGACTT | 79 | 134 |
| 1146089 | N/A | N/A | 8878 | 8893 | AGTCTAAGATTGATAC | 65 | 135 |
| 1146097 | N/A | N/A | 10010 | 10025 | AAATTTGTGAGCTACA | 43 | 136 |
| 1146101 | N/A | N/A | 10122 | 10137 | AATAGTAAGGAATTGG | 69 | 137 |
| 1146109 | N/A | N/A | 10304 | 10319 | TAGTATATAGGGTCCC | 48 | 138 |
| 1146113 | N/A | N/A | 10742 | 10757 | GCAATATTGTCAAGGG | 42 | 139 |
| 1146117 | N/A | N/A | 11143 | 11158 | GTTATTAGGTGGATTC | 74 | 140 |
| 1146121 | N/A | N/A | 11184 | 11199 | GATCTTAAGGTCCACG | 46 | 141 |
| 1146125 | N/A | N/A | 11670 | 11685 | CAGGGATATGCTGCAG | 46 | 142 |
| 1146129 | N/A | N/A | 12236 | 12251 | GTAGGGTTGTGTTTGC | 63 | 143 |
| 1146133 | N/A | N/A | 12454 | 12469 | CTTCTTAATCAGGTTT | 46 | 144 |
| 1146137 | N/A | N/A | 13306 | 13321 | GTGCGATTGTGATGCC | 59 | 145 |
| 1146145 | N/A | N/A | 15001 | 15016 | ACATTCGAGATGCACA | 43 | 146 |
| 1146149 | N/A | N/A | 15544 | 15559 | GTGCGATTTCTACAGA | 70 | 147 |
| 1146153 | N/A | N/A | 15925 | 15940 | GCAAAATTGGATGACG | 53 | 148 |
| 1146157 | N/A | N/A | 16150 | 16165 | CGCTTTTAAGGCACGC | 17 | 149 |
| 1146165 | N/A | N/A | 17579 | 17594 | AAAATTATATGCTCCG | 68 | 150 |
| 1146169 | N/A | N/A | 18289 | 18304 | GTAGATTAAAAGGTGA | 73 | 151 |
| 1146173 | N/A | N/A | 18559 | 18574 | GAATTCTATGGTGTCT | 88 | 152 |
| 1146177 | N/A | N/A | 18715 | 18730 | AAGAATACAGGACTTC | 74 | 153 |
| 1146181 | N/A | N/A | 20226 | 20241 | ATCAAGGAACAACCAG | 73 | 154 |
| 1146185 | N/A | N/A | 21737 | 21752 | GCTAGTAATGACTTTC | 81 | 155 |
| 1146189 | N/A | N/A | 22268 | 22283 | AACTATACATGGCTCT | 79 | 156 |
| 1146193 | N/A | N/A | 22452 | 22467 | GCATATATTAATGGTT | 92 | 157 |
| 1146197 | N/A | N/A | 22561 | 22576 | AATTTTTAACCTACGC | 62 | 158 |
| 1146201 | N/A | N/A | 22745 | 22760 | GCAGAGGTAATCATGC | 83 | 159 |

TABLE 4
Percent control of HSD17B13 mRNA with 3-10-3 cEt gapmers
with phosphorothioate internucleoside linkages

| | SEQ | SEQ | SEQ | SEQ | | | |
|---|---|---|---|---|---|---|---|
| | ID No: | ID No: | ID No: | ID No: | | HSD17B13 | SEQ |
| Compound | 1 Start | 1 Stop | 2 Start | 2 Stop | | (% | ID |
| Number | Site | Site | Site | Site | Sequence (5′ to 3′) | control) | NO |
| 1145894 | 2 | 17 | 2614 | 2629 | GTATTATGGGCTGCTG | 85 | 160 |
| 1145906 | 181 | 196 | 2793 | 2808 | GATGAGAACGGTCTGC | 42 | 161 |
| 1145918 | 320 | 335 | 7363 | 7378 | GCACGACGGCCCCCAG | n.d. | 162 |
| 1145922 | 361 | 376 | 7404 | 7419 | GTAAATCTCGGCCCGG | n.d. | 163 |
| 1145926 | 405 | 420 | 9121 | 9136 | ATCTCGACATCACCTA | n.d. | 164 |
| 1145930 | 433 | 448 | 9149 | 9164 | ATATATCGCCCCGGCG | 52 | 165 |
| 1145938 | 451 | 466 | 9167 | 9182 | ACTAAGAAGGTCTGCT | 33 | 166 |
| 1145962 | 652 | 667 | 12914 | 12929 | TGCTCGGTGGAAGCCC | 61 | 167 |
| 1146006 | 1061 | 1076 | 23890 | 23905 | GTAATAGTAGCACATT | 102 | 168 |
| 1146010 | 1076 | 1091 | 23905 | 23920 | TGGCTTAAAACATCAG | 82 | 169 |
| 1146014 | 1158 | 1173 | 23987 | 24002 | TTATACAGTCAGAGTG | 34 | 170 |
| 1146026 | 1427 | 1442 | 24256 | 24271 | AAGGCATGGGTACAAT | 110 | 171 |
| 1146030 | 1436 | 1451 | 24265 | 24280 | TCATATACCAAGGCAT | 135 | 172 |
| 1146034 | N/A | N/A | 2892 | 2907 | ACCTATTATTACCTTA | 57 | 173 |
| 1146038 | N/A | N/A | 3487 | 3502 | GAGCGGTTGCTCGGTG | 63 | 174 |
| 1146046 | N/A | N/A | 3909 | 3924 | AGATTACAATCCCAGA | 53 | 175 |
| 1146050 | N/A | N/A | 3982 | 3997 | GATCTAATATCTAGTT | 57 | 176 |
| 1146054 | N/A | N/A | 4451 | 4466 | ATTTTTTACGAGGGAC | 47 | 177 |
| 1146058 | N/A | N/A | 4538 | 4553 | GTATTGATGTCTTCCC | 55 | 178 |
| 1146062 | N/A | N/A | 5099 | 5114 | TGAACGATGTCCTGTG | 43 | 179 |
| 1146066 | N/A | N/A | 5994 | 6009 | CCACTAATGAAGGCTG | 47 | 180 |
| 1146070 | N/A | N/A | 6554 | 6569 | CTTACTATGTGAACCC | 46 | 181 |
| 1146074 | N/A | N/A | 7035 | 7050 | GTTATCGAAGATGCTG | 57 | 182 |
| 1146078 | N/A | N/A | 7185 | 7200 | TTAACTAATTAGCTGG | 67 | 183 |
| 1146082 | N/A | N/A | 7960 | 7975 | CATACCTAACAACCCC | 58 | 184 |
| 1146086 | N/A | N/A | 8733 | 8748 | AGATATCGATCTGACT | 73 | 185 |
| 1146094 | N/A | N/A | 9739 | 9754 | ATCAGTAAACCTTTAC | 87 | 186 |
| 1146102 | N/A | N/A | 10180 | 10195 | CATACAAATGCTCCCT | 58 | 187 |
| 1146114 | N/A | N/A | 10863 | 10878 | AGAATTAAGTAGCTCC | 55 | 188 |
| 1146118 | N/A | N/A | 11147 | 11162 | CACGGTTATTAGGTGG | 105 | 189 |
| 1146122 | N/A | N/A | 11363 | 11378 | ATTATAATAATCCCTA | 97 | 190 |
| 1146126 | N/A | N/A | 12111 | 12126 | AGCTATATAAGTTTTA | 91 | 191 |
| 1146130 | N/A | N/A | 12276 | 12291 | ACAACGAGTATTTGGA | 41 | 192 |
| 1146138 | N/A | N/A | 13405 | 13420 | GGATTTTAGATGTCAC | 58 | 193 |
| 1146146 | N/A | N/A | 15286 | 15301 | GTAACAAATCACCCAC | 49 | 194 |
| 1146150 | N/A | N/A | 15606 | 15621 | GGAGGGTTAGGTCTGA | 144 | 195 |
| 1146154 | N/A | N/A | 15999 | 16014 | TTATTGTTTAGTCTCC | 44 | 196 |
| 1146158 | N/A | N/A | 16386 | 16401 | ATCTATAAGTATAGGA | 5 | 197 |
| 1146162 | N/A | N/A | 17455 | 17470 | GCAATATCATATTCTA | 32 | 198 |
| 1146170 | N/A | N/A | 18349 | 18364 | GATAGATCAATCACAA | 4 | 199 |
| 1146174 | N/A | N/A | 18637 | 18652 | TTTAATCTCTTTAGTG | 102 | 200 |
| 1146178 | N/A | N/A | 19634 | 19649 | GCAGTATACAAGAGGT | 78 | 201 |
| 1146182 | N/A | N/A | 20359 | 20374 | CATACCCAAATACGGC | 102 | 202 |
| 1146186 | N/A | N/A | 21900 | 21915 | GTACTAATGTTGCCTT | 90 | 203 |
| 1146190 | N/A | N/A | 22311 | 22326 | CCTACAAAATTGGAGA | 96 | 204 |
| 1146194 | N/A | N/A | 22453 | 22468 | AGCATATATTAATGGT | 86 | 205 |
| 1146198 | N/A | N/A | 22611 | 22626 | AGATTAATGCTAGAGG | 102 | 206 |
| 1146202 | N/A | N/A | 23331 | 23346 | GGAGATACTGGCCGCC | 81 | 207 |

### Example 2: Effect of 3-10-3 cEt Gapmers with Phosphorothioate Internucleoside Linkages on HSD17B13 In Vitro, Multiple Doses

Modified oligonucleotides selected from the example above were tested at various doses in mouse primary hepatocyte cells. Cells were plated at a density of 20,000 cells per well and transfected using electroporation with 222.2 nM, 666.6 nM, 2,000 nM, and 6,000 nM concentrations of modified oligonucleotide, as specified in the tables below. After a treatment period of approximately 24 hours, total RNA was isolated from the cells and HSD17B13 mRNA levels were measured by quantitative real-time PCR. Mouse HSD17B13 primer probe set RTS40764 (described in Example 1) was used to measure mRNA levels. HSD17B13 mRNA levels were adjusted according to total RNA content, as measured by RIBOGREEN®. Results are presented in the tables below as percent control of the amount of HSD17B13 mRNA, relative to untreated control cells. As illustrated in the tables below, HSD17B13 mRNA levels were reduced in a dose-dependent manner in modified oligonucleotide-treated cells.

TABLE 5
Dose-dependent percent reduction of HSD17B13
mRNA by modified oligonucleotides

| Compound | HSD17B13 expression (% control) | | | |
|---|---|---|---|---|
| Number | 222.2 nM | 666.6 nM | 2,000 nM | 6,000 nM |
| 1145905 | 44 | 41 | 39 | 50 |
| 1145909 | 33 | 29 | 30 | 38 |
| 1145933 | 36 | 37 | 38 | 41 |
| 1145937 | 41 | 26 | 42 | 33 |
| 1145938 | 42 | 41 | 37 | 33 |
| 1145966 | 35 | 33 | 31 | 27 |
| 1145969 | 41 | 43 | 32 | 42 |
| 1145973 | 33 | 33 | 30 | 37 |
| 1145981 | 62 | 56 | 65 | 60 |
| 1145986 | 38 | 39 | 35 | 30 |
| 1145990 | 29 | 22 | 22 | 32 |
| 1145993 | 38 | 36 | 31 | 37 |
| 1145997 | 30 | 28 | 33 | 41 |
| 1145998 | 22 | 32 | 20 | 24 |
| 1146014 | 85 | 60 | 46 | 37 |
| 1146157 | 25 | 18 | 18 | 18 |
| 1146158 | 5 | 4 | 3 | 3 |
| 1146162 | 57 | 53 | 37 | 24 |
| 1146170 | 20 | 7 | 5 | 3 |

TABLE 6
Dose-dependent percent reduction of HSD17B13
mRNA by modified oligonucleotides

| Compound | HSD17B13 expression (% control) | | | |
|---|---|---|---|---|
| Number | 222.2 nM | 666.6 nM | 2,000 nM | 6,000 nM |
| 1145900 | 27 | 23 | 19 | 22 |
| 1145903 | 45 | 27 | 41 | 46 |
| 1145936 | 29 | 33 | 22 | 15 |
| 1145940 | 39 | 29 | 26 | 25 |
| 1145960 | 51 | 52 | 58 | 45 |
| 1145963 | 25 | 27 | 24 | 29 |
| 1145967 | 29 | 22 | 28 | 31 |
| 1145968 | 34 | 33 | 30 | 26 |
| 1145976 | 38 | 38 | 31 | 27 |
| 1145979 | 39 | 34 | 36 | 37 |
| 1145983 | 44 | 41 | 30 | 44 |
| 1145987 | 28 | 31 | 24 | 22 |
| 1145991 | 31 | 27 | 29 | 36 |
| 1145992 | 52 | 36 | 35 | 31 |
| 1145995 | 46 | 32 | 28 | 38 |
| 1145996 | 20 | 27 | 18 | 18 |
| 1146067 | 63 | 58 | 56 | 57 |
| 1146156 | 29 | 36 | 29 | 25 |
| 1146157 | 20 | 15 | 16 | 14 |

### Example 4: ASO Inhibition of HSD17B13 in Male Gubra Mice

Lep^{ob}/Lep^{ob} mice fed a high fat/fructose/cholesterol diet is known herein as the “Gubra” mouse model (Gubra ApS, Horshom, Denmark). The Gubra mouse is an accelerated diet-induced obese mouse model for fatty liver disease including fatty liver, NASH, and fibrosis. The Gubra mouse exhibits elements of liver steatosis, ballooning degeneration of hepatocytes, inflammation and fibrosis and affects metabolic parameters including body weight, hyperinsulinemia, fasting hyperleptinemia and impaired glucose tolerance.

To develop the diet induced Gubra phenotype, five-week old male mice are fed a high fat/fructose/cholesterol diet (40% HFD, 18% fructose, 2% cholesterol) for 19 weeks prior to the start of the study. After 16-17 weeks on the diet, the mice are pre-screened and randomized into treatment groups after liver biopsy and histological assessment (e.g., scoring of fibrosis after staining with Sirius Red and steatosis after staining with H&E).

### Treatment

Antisense oligonucleotides targeting HSD17B13 will be administered to Gubra mice to test its effects on the mice. After 19-weeks on the high fat/fructose/cholesterol diet, a group of mice will be treated with subcutaneous weekly injections of oligonucleotide or PBS control over the course of eight weeks.

At the end of the study (8-weeks), the mice will be sacrificed and livers removed at the time of sacrifice. Liver RNA will be extracted, as well as liver TG and TC, and analyses of hepatic pathology including steatosis, fibrosis stage and NAFLD Activity Score (NAS) will be assessed and compared to pre-study biopsies at baseline. RNA will be extracted from liver for real-time PCR analysis of liver HSD17B13 RNA levels.

### Body and Organ Weights

Body weights of the Gubra mice will be measured every two days, and liver, left and right kidneys, and spleen weights will be measured at week 8, at the end of the study. Also at the end of the study, EchoMRT scanning and terminal necropsy will be performed, organs (liver, kidney, spleen, epididymal adipose tissue and quadriceps muscle) will be harvested and liver, kidney and spleen weights will be measured. Averages for each treatment group will be calculated.

### Plasma Chemistry Markers

To evaluate the effect of ASOs on hepatic function, plasma concentrations of liver transaminases ALT and AST, as well as plasma lipids (TG and TC) will be measured in Gubra mice at baseline and at the end of the 8-week study.

### Glucose Tolerance

An Oral Glucose Tolerance Test (OGTT) will be performed. At week four of the eight week study, 4-hour fasted mice will be subcutaneously injected with antisense oligonucleotide targeting HSD17B13 at time=0 minus one hour, and blood glucose will be measured. One hour later, at time 0, glucose (2 g/kg) will be ingested by the mice and blood glucose will be tested again; thereafter, blood glucose will be tested at 15 min, 30 min, 60 min and 120 min time points. Blood glucose area under the curve (AUC) will be also calculated mmol/L×minute.

### Fibrosis Markers

Liver TG and liver TC content (mg/g of liver) will be also assayed by biochemical analysis.

Liver fibrosis markers hydroxyproline and collagen will be assessed in the mice. Liver collagen mRNA levels will be quantified using a Col1a2 assay (ThermoFisher Scientific assay ID #Mm00483888_m1).

Overall, data from this Gubra mouse study will indicate that an ASO targeting HSD17B13 is active in liver, well tolerated, will decrease several biomarkers of metabolic and liver diseases, and HSD17B13 is an important candidate for the treatment of obesity, type 2 diabetes and/or insulin sensitivity, hyperlipidemia, NASH, and NAFLD diseases, disorders or conditions.

### Example 5: ASO Inhibition of HSD17B13 in Male Ob/Ob Mice

ASOs described in the studies above will be evaluated for their ability to reduce murine HSD17B13 RNA transcript in an 8-week ob/ob mice study.

### Treatment

C57BL/6J-Lepr ob (“ob/ob”) mice will be divided into treatment groups. Mice will be injected subcutaneously once a week for 8 weeks with control oligonucleotide, or antisense oligonucleotides targeting HSD17B13, and one group of ob/ob mice will be injected with PBS as a control to which the antisense oligonucleotide treated groups are compared. Several clinical endpoints will be measured over the course of the study. The body and food weights will be measured weekly, and tail bleeds will be performed at baseline and weekly thereafter, as well as at the time of sacrifice. The mice will be euthanized 72 hours after the last dose and after 8 weeks of ASO treatment organs and plasma will be harvested for further analysis.

### RNA Analysis

At the end of the treatment period, RNA will be extracted from liver, kidney, white adipose tissue (WAT) and pancreas for quantitative real-time PCR analysis of RNA expression of HSD17B13.

### Plasma Chemistry Markers

To evaluate the effect of treatment with ISIS oligonucleotides on plasma levels of various biomarkers of liver and kidney function, plasma levels of liver transaminases (ALT and AST), total cholesterol (CHOL), creatinine (CRE), glucose (GLU), HDL, LDL, triglycerides (TRIG), BUN, non-esterified fatty acids (NEFA), 3HB will be measured using an automated clinical chemistry analyzer (Hitachi Olympus AU400e, Melville, N.Y.).

### Hepatic Triglycerides

Hepatic triglyceride (TG) concentrations (μg/g of liver) will be assayed by ELISA. Significant reduction in liver TG levels will indicate that HSD17B13 ASOs may be effective in reducing or preventing fatty liver diseases such as hepatic steatosis, NASH and/or NAFLD.

### Example 6: Activity of Modified Oligonucleotides Targeting Mouse HSD17B13 in Lean C57BL/6 Mice at 4 Weeks

C57BL/6 mice (Jackson Laboratory) are a multipurpose mouse model frequently utilized for safety and efficacy testing. The mice were treated with modified oligonucleotides selected from studies described above and evaluated for changes in the levels of various plasma chemistry markers, as well as for efficacy of modified oligonucleotide mediated knockdown of target RNA in the liver.

### Treatment

Groups of 6-week-old male C57BL/6 mice were injected subcutaneously once a week for 4 weeks (a total of 4 treatments) with 50 mg/kg of modified oligonucleotide. One group of male C57BL/6 mice was injected with PBS. One group of mice was injected with ION No. 549144 (3-10-3 cET gapmer, GGCCAATACGCCGTCA, designated herein as SEQ ID NO: 208), a control modified oligonucleotide that does not target HSD17B13, as a negative control. Mice were euthanized 48 hours following the final administration.

### Plasma Chemistry Markers

To evaluate the effect of modified oligonucleotides on liver function, plasma levels of albumin (ALB), alanine aminotransferase (ALT), aspartate aminotransferase (AST), and total bilirubin (TBIL) were measured using an automated clinical chemistry analyzer (Hitachi Olympus AU400c, Melville, N.Y.). The results are presented in the table below.

TABLE 7
Plasma chemistry markers in male C57BL/6 mice

| | ALT | AST | TBIL |
|---|---|---|---|
| ION No | (IU/L | (IU/L) | (mg/dL) |
| PBS | 38 | 60 | 0.15 |
| 549144 | 30 | 45 | 0.23 |
| 1145963 | 49 | 62 | 0.20 |
| 1145967 | 70 | 77 | 0.15 |
| 1145973 | 68 | 77 | 0.23 |
| 1145991 | 158 | 184 | 0.20 |
| 1146156 | 40 | 50 | 0.15 |
| 1146157 | 49 | 75 | 0.23 |
| 1146158 | 100 | 131 | 0.15 |
| 1146170 | 61 | 88 | 0.15 |

### Body and Organ Weights

Body weights of C57BL/6 mice were measured at day 25 (4 weeks post 1″ dose), and the average body weight for each group is presented in the table below. Kidney, spleen, and liver weights were measured at the end of the study and are presented in the table below.

TABLE 8
Body and organ weights (in grams)

| | Body Weight | Liver | Kidney | Spleen |
|---|---|---|---|---|
| ION No. | (g) | (g) | (g) | (g) |
| PBS | 27 | 1.29 | 0.32 | 0.08 |
| 549144 | 26 | 1.34 | 0.34 | 0.10 |
| 1145963 | 27 | 1.44 | 0.30 | 0.08 |
| 1145967 | 27 | 1.48 | 0.32 | 0.07 |
| 1145973 | 25 | 1.29 | 0.29 | 0.10 |
| 1145991 | 27 | 1.36 | 0.32 | 0.09 |
| 1146156 | 28 | 1.58 | 0.33 | 0.10 |
| 1146157 | 28 | 1.42 | 0.31 | 0.08 |
| 1146158 | 28 | 1.39 | 0.34 | 0.11 |
| 1146170 | 28 | 1.29 | 0.33 | 0.09 |

### RNA Analysis

On day 30, RNA was extracted from livers for real-time RTPCR analysis of HSD17B13 RNA expression. Primer probe set RTS40764 was used to measure mouse HSD17B13 mRNA levels. HSD17B13 mRNA levels were normalized to total RNA content, as measured by RIBOGREEN®. In addition, HSD17B13 mRNA levels were normalized to mouse cyclophilin A. Results are presented as percent inhibition of HSD17B13 relative to untreated control cells. As used herein, a value of ‘0’ indicates that treatment with the modified oligonucleotide did not inhibit HSD17B13 mRNA levels.

As presented in the table below, treatment with Ionis modified oligonucleotides resulted in significant reduction of HSD17B13 mRNA in comparison to the PBS control.

TABLE 9
Modified oligonucleotide-mediated inhibition
of mouse HSD17B13 in C57BL/6 mice

% inhibition

| | Normalized to | Normalized to |
|---|---|---|
| ION No. | Ribogreen | cyclophilin A |
| 549144 | 0 | 0 |
| 1145963 | 85 | 84 |
| 1145967 | 99 | 99 |
| 1145973 | 94 | 94 |
| 1145991 | 97 | 96 |
| 1146156 | 82 | 79 |
| 1146157 | 92 | 90 |
| 1146158 | 89 | 85 |
| 1146170 | 0 | 0 |

### Protein Analysis

Protein analysis was carried out on liver samples from animals treated with ION No. 1146157 using standard procedures. HSD17B13 protein levels in livers of animals treated with control modified oligonucleotide and with PBS were also tested. HSD17B13 levels were detected using rabbit anti-HSD17B13 polyclonal antibody, PA5-25633 (ThermoFisher) as the primary antibody and anti-rabbit IgG, HRP-linked antibody, 7074 (Cell Signaling Technology) as the secondary antibody. HSD17B13 protein levels were normalized to internal control GAPDH.

TABLE 10
Quantitative analysis of protein levels

| | Protein concentration |
|---|---|
| ION No. | (% control) |
| 549144 | 124 |
| 1146157 | 4 |

### Example 7: Activity of Modified Oligonucleotides Targeting Mouse HSD17B13 in Lean CD-1 (CRL) Mice at 8 Weeks

CD-1 (CRL) mice (Charles River) are a multipurpose mouse model frequently utilized for safety and efficacy testing. The mice were treated with modified oligonucleotides selected from studies described above and evaluated for changes in the levels of various plasma chemistry markers, as well as for efficacy of modified oligonucleotide mediated knockdown of target RNA in the liver.

ION No. 1251684 is a 3-10-3 cET gapmer, with the same sequence as 1146157 (SEQ IS NO: 149). ION No. 549144 was been included as a negative control. ION No. 740133, also added as a negative control, is a 3-10-3 cET gapmer, with the same sequence as 549144 (SEQ ID NO: 208). Ion Nos. 1251684 and 740133 are each conjugated with a THA-GalNAc conjugate group at the 5′-end. THA-GalNac refers to this structure:

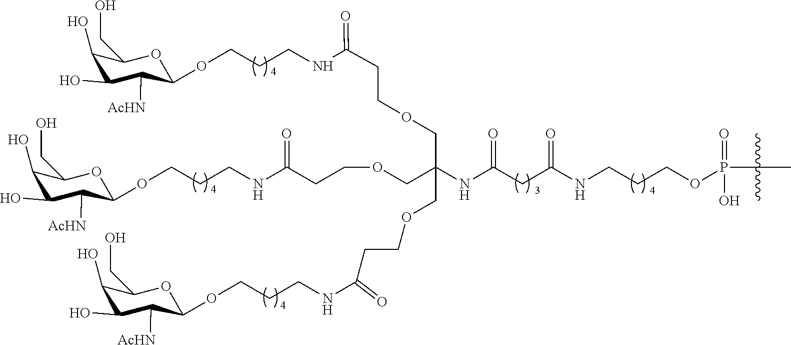

wherein the phosphate group is attached to the 5′-oxygen atom of the 5′ nucleoside.

### Treatment

Groups of four 6-week-old male and four 6-week old female CD-1 mice were injected subcutaneously once a week for 8 weeks (a total of 9 treatments) with 50 and 10 mg/kg of unconjugated modified oligonucleotides, Ion Nos. 549144 or 1146157, or 5 and 2.5 mg/kg of conjugated modified oligonucleotides, Ion Nos. 740133 or 1251684.

One group each of 4 male and 4 female CD-1 mice was injected with PBS. Mice were euthanized 8 weeks post 1^{st} administration (48 hours following the final administration).

### Plasma Chemistry Markers

To evaluate the effect of modified oligonucleotides on liver function, plasma levels of alanine aminotransferase (ALT), aspartate aminotransferase (AST), total bilirubin (TBIL), and plasma triglycerides (TRIG) were measured using an automated clinical chemistry analyzer (Hitachi Olympus AU400c, Melville, N.Y.). The results are presented in the table below.

TABLE 11
Plasma chemistry markers in CD1 mice

| | | Concen- | | | | |
|---|---|---|---|---|---|---|
| | | tration | ALT | AST | TBIL | TRIG |
| Sex | ION No. | (mpk) | (IU/L) | (IU/L) | (mg/dL) | (mg/dL) |
| Male | PBS | | 21 | 45 | 0.26 | 200 |
| | 549144 | 50 | 27 | 43 | 0.19 | 171 |
| | 740133 | 5 | 26 | 45 | 0.17 | 306 |
| | 1146157 | 50 | 33 | 61 | 0.19 | 100 |
| | 1146157 | 10 | 69 | 209 | 0.18 | 178 |
| | 1251684 | 5 | 31 | 47 | 0.2 | 75 |
| | 1251684 | 2.5 | 23 | 40 | 0.17 | 181 |
| Female | PBS | | 21 | 52 | 0.18 | 150 |
| | 549144 | 50 | 26 | 51 | 0.18 | 170 |
| | 740133 | 5 | 32 | 59 | 0.19 | 167 |
| | 1146157 | 50 | 37 | 73 | 0.19 | 91 |
| | 1146157 | 10 | 28 | 70 | 0.18 | 105 |
| | 1251684 | 5 | 23 | 54 | 0.19 | 67 |
| | 1251684 | 2.5 | 21 | 60 | 0.18 | 97 |

### Body and Organ Weights

Body weights of CD-1 mice were measured at day 58 (8 weeks post 1^{st} dose), and the average body weight for each group is presented in the table below. Kidney, spleen, and liver weights were measured at the end of the study and are presented in the table below.

TABLE 12
Body and organ weights (in grams)

| | | Concen- | | | | |
|---|---|---|---|---|---|---|
| | | tration | Body Weight | Liver | Kidney | Spleen |
| Sex | ION No. | (mpk) | (g) | (g) | (g) | (g) |
| Male | PBS | | 43 | 2.1 | 0.56 | 0.11 |
| | 549144 | 50 | 45 | 2.1 | 0.53 | 0.14 |
| | 740133 | 5 | 43 | 2.0 | 0.52 | 0.11 |
| | 1146157 | 50 | 44 | 2.3 | 0.58 | 0.16 |
| | 1146157 | 10 | 49 | 2.5 | 0.62 | 0.15 |
| | 1251684 | 5 | 46 | 2.1 | 0.56 | 0.12 |
| | 1251684 | 2.5 | 43 | 2.2 | 0.55 | 0.12 |
| Female | PBS | | 32 | 1.4 | 0.37 | 0.16 |
| | 549144 | 50 | 35 | 1.6 | 0.34 | 0.13 |
| | 740133 | 5 | 32 | 1.5 | 0.37 | 0.14 |
| | 1146157 | 50 | 35 | 1.7 | 0.40 | 0.16 |
| | 1146157 | 10 | 32 | 1.5 | 0.37 | 0.15 |
| | 1251684 | 5 | 32 | 1.5 | 0.38 | 0.15 |
| | 1251684 | 2.5 | 37 | 1.5 | 0.36 | 0.14 |

### RNA Analysis

RNA was extracted from livers for real-time RTPCR analysis of HSD17B13 RNA expression. Primer probe set RTS40764 was used to measure mouse HSD17B13 mRNA levels. HSD17B13 mRNA levels were normalized to mouse cyclophilin A, measured by mouse primer-probe set m_cyclo24. Results are presented as percent inhibition of HSD17B13 relative to untreated control cells. As used herein, a value of ‘0’ indicates that treatment with the modified oligonucleotide did not inhibit HSD17B13 mRNA levels.

As presented in the table below, treatment with Ionis modified oligonucleotides resulted in significant reduction of HSD17B13 mRNA in comparison to the PBS control.

TABLE 13
Modified oligonucleotide-mediated inhibition
of mouse HSD17B13 in CD1 mice

| | | Concentration | % Inhibition |
|---|---|---|---|
| Sex | ION No. | (mpk) | HSD17B13 |
| Male | 549144 | 50 | 20 |
| | 740133 | 5 | 5 |
| | 1146157 | 50 | 93 |
| | 1146157 | 10 | 71 |
| | 1251684 | 5 | 91 |
| | 1251684 | 2.5 | 88 |
| Female | 549144 | 50 | 0 |
| | 740133 | 5 | 0 |
| | 1146157 | 50 | 93 |
| | 1146157 | 10 | 75 |
| | 1251684 | 5 | 95 |
| | 1251684 | 2.5 | 90 |

What is claimed is:

1. A method of treating, preventing, delaying the onset, slowing the progression, or ameliorating a liver disease or disorder in an individual having, or at risk of having, a liver disease or disorder comprising administering an HSD17B13 specific inhibitor to the individual, thereby treating, preventing, delaying the onset, slowing the progression, or ameliorating the liver disease or disorder in the individual.

2. The method of claim 1, wherein the liver disease or disorder is fatty liver disease, chronic liver disease, liver cirrhosis, hepatic steatosis, steatohepatitis, nonalcoholic fatty liver disease (NAFLD), or nonalcoholic steatohepatitis (NASH).

3. The method of claim 1 or 2, wherein the HSD17B13 specific inhibitor reduces or improves hepatic steatosis, liver fibrosis, triglyceride synthesis, lipid levels, hepatic lipids, ALT levels, NAFLD Activity Score (NAS), cholesterol levels, or triglyceride levels.

4. A method of inhibiting expression or activity of HSD17B13 in a cell comprising contacting the cell with an HSD17B13 specific inhibitor, thereby inhibiting expression or activity of HSD17B13 in the cell.

5. The method of claim 4, wherein the cell is a hepatocyte.

6. The method of claim 5, wherein the cell is in an individual.

7. The method of claim 6, wherein the individual has, or is at risk of having liver disease, fatty liver disease, chronic liver disease, liver cirrhosis, hepatic steatosis, steatohepatitis, nonalcoholic fatty liver disease (NAFLD), or nonalcoholic steatohepatitis (NASH).

8. The method of any preceding claim, wherein the individual is human.

9. The method of any preceding claim, wherein the HSD17B13 specific inhibitor is selected from a nucleic acid, a polypeptide, an antibody, and a small molecule.

10. The method of any preceding claim, wherein the HSD17B13 specific inhibitor comprises a modified oligonucleotide, wherein the modified oligonucleotide has a nucleobase sequence complementary to any one of SEQ ID NOs: 1-6.

11. The method of claim 10, wherein the modified oligonucleotide is single-stranded.

12. The method of claim 10, wherein the modified oligonucleotide is double-stranded.

13. The method of any one of claims 10-12, wherein the modified oligonucleotide consists of 12 to 30 linked nucleosides.

14. The method of claim 13, wherein at least one of the nucleosides comprise a modified sugar moiety.

15. The method of claim 13 or claim 14, wherein at least one of the nucleosides comprise a modified nucleobase.

16. The method of any one of claims 13-15, wherein at least one internucleoside linkage of the modified oligonucleotide is a modified internucleoside linkage.

17. The method of claim 14, wherein the modified sugar is a bicyclic sugar or 2′-O-methyoxyethyl.

18. The method of claim 14, wherein the modified sugar comprises a 4′-CH(CH₃)—O-2′ bridge or a 4′-(CH₂)ₙ—O-2′ bridge, wherein n is 1 or 2.

19. The method of claim 15, wherein the modified nucleobase is a 5-methylcytosine.

20. The method of claim 16, wherein the at least one modified internucleoside linkage is a phosphorothioate internucleoside linkage.

21. The method of any one of claims 10-20, wherein the modified oligonucleotide has:
a gap segment consisting of linked deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar.

22. The method of any of the preceding claims, wherein the HSD17B13 specific inhibitor is administered parenterally.

23. The method of claim 18, wherein the compound is administered parenterally by subcutaneous or intravenous administration.

24. The method of any of the preceding claims, comprising co-administering the compound and at least one additional therapy.

25. Use of an HSD17B13 specific inhibitor for the manufacture or preparation of a medicament for treating a liver disease or disorder.

26. Use of an HSD17B13 specific inhibitor for the treatment of a liver disease or disorder.

27. The use of claim 25 or 26, wherein the liver disease or disorder is fatty liver disease, chronic liver disease, liver cirrhosis, hepatic steatosis, steatohepatitis, nonalcoholic fatty liver disease (NAFLD), or nonalcoholic steatohepatitis (NASH).

28. The use of any of claims 25-27, wherein the HSD17B13 specific inhibitor reduces or improves hepatic steatosis, liver fibrosis, triglyceride synthesis, lipid levels, hepatic lipids, ALT levels, NAFLD Activity Score (NAS), cholesterol levels, or triglyceride levels.

29. The use of any of claims 25-28, wherein the HSD17B13 specific inhibitor is selected from a nucleic acid, a polypeptide, an antibody, and a small molecule.

30. The use of any of claims 25-29, wherein the HSD17B13 specific inhibitor comprises a modified oligonucleotide, wherein the modified oligonucleotide has a nucleobase sequence complementary to any one of SEQ ID NOs: 1-6.

31. The use of claim 30, wherein the modified oligonucleotide is single-stranded.

32. The use of claim 30, wherein the modified oligonucleotide is double-stranded

33. The use of any one of claims 30-32, wherein the modified oligonucleotide consists of 12 to 30 linked nucleosides.

34. The use of claim 33, wherein at least one of the nucleosides comprise a modified sugar moiety.

35. The use of claim 33 or claim 34, wherein at least one of the nucleosides comprise a modified nucleobase.

36. The use of any one of claims 33-35, wherein at least one internucleoside linkage of the modified oligonucleotide is a a modified internucleoside linkage.

37. The method of claim 34, wherein the modified sugar is a bicyclic sugar or 2′-O-methyoxyethyl.

38. The method of claim 34, wherein the modified sugar comprises a 4′-CH(CH₃)—O-2′ bridge or a 4′-(CH₂)ₙ—O-2′ bridge, wherein n is 1 or 2.

39. The method of claim 35, wherein the modified nucleobase is a 5-methylcytosine.

40. The method of claim 36, wherein the at least one modified internucleoside linkage is a phosphorothioate internucleoside linkage.

41. The use of any one of claims 30-40, wherein the modified oligonucleotide has:
a gap segment consisting of linked deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar.

42. A method comprising administering an HSD17B13 specific inhibitor to an individual.

43. The method of claim 42, wherein the individual has a liver disease or is at risk for developing a liver disease.

44. The method of claim 43, wherein the liver disease is selected from fatty liver disease, chronic liver disease, liver cirrhosis, hepatic steatosis, steatohepatitis, nonalcoholic fatty liver disease (NAFLD), and nonalcoholic steatohepatitis (NASH).

45. The method of claim 43 or 44, wherein a therapeutic amount of the HSD17B13 specific inhibitor is administered to the individual.

46. The method any of claims 43-45, wherein the administration of the HSD17B13 specific inhibitor results in the prevention, delay, slowed progression, and/or amelioration of at least one symptom of the liver disease.

47. The method of any of claims 42-46, wherein the administration of the HSD17B13 specific inhibitor reduces, improves, or regulates hepatic steatosis, liver fibrosis, triglyceride synthesis, lipid levels, hepatic lipids, ALT levels, NAFLD Activity Score (NAS), cholesterol levels, or triglyceride levels.

48. A method comprising contacting a cell with an HSD17B13 specific inhibitor.

49. The method of claim 48, wherein expression of HSD17B13 in the cell is reduced.

50. The method of claim 48 or 49, wherein the cell is a hepatocyte.

51. The method of claim 50, wherein the cell is in an individual.

52. The method of claim 51, wherein the individual has, or is at risk of having liver disease, fatty liver disease, chronic liver disease, liver cirrhosis, hepatic steatosis, steatohepatitis, nonalcoholic fatty liver disease (NAFLD), or nonalcoholic steatohepatitis (NASH).

53. The method of any preceding claim, wherein the individual is human.

54. The method of any preceding claim, wherein the HSD17B13 specific inhibitor comprises or consists of a nucleic acid, a polypeptide, an antibody, or a small molecule.

55. The method of any preceding claim, wherein the HSD17B13 specific inhibitor comprises a modified oligonucleotide, wherein the modified oligonucleotide has a nucleobase sequence complementary to any one of SEQ ID NOs: 1-6.

56. The method of claim 55, wherein the modified oligonucleotide is single-stranded.

57. The method of claim 55, wherein the modified oligonucleotide is double-stranded.

58. The method of any of claims 55-57, wherein the modified oligonucleotide consists of 12 to 30 linked nucleosides.

59. The method of claim 58, wherein at least one nucleoside of the modified oligonucleotide comprises a modified sugar moiety.

60. The method of claim 59, wherein the modified sugar moiety is a bicyclic sugar moiety or a sugar moiety comprising a 2′-O-methyoxyethyl.

61. The method of claim 59, wherein the modified sugar comprises a 4′-CH(CH₃)—O-2′ bridge or a 4′-(CH₂)ₙ—O-2′ bridge, wherein n is 1 or 2.

62. The method of any of claims 58-61, wherein at least one nucleoside of the modified oligonucleotide comprises a modified nucleobase.

63. The method of claim 62, wherein the modified nucleobase is a 5-methylcytosine.

64. The method of any one of claims 58-63, wherein at least one internucleoside linkage of the modified oligonucleotide is a modified internucleoside linkage.

65. The method of claim 64, wherein the at least one modified internucleoside linkage is a phosphorothioate internucleoside linkage.

66. The method of any one of claims 55-65, wherein the modified oligonucleotide has:
a gap segment consisting of linked deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar.

67. The method of any of the preceding claims, wherein the HSD17B13 specific inhibitor is administered parenterally.

68. The method of claim 67, wherein the HSD17B13 specific inhibitor is administered parenterally by subcutaneous or intravenous administration.

69. The method of any of the preceding claims, comprising co-administering the HSD17B13 specific inhibitor and at least one additional therapy.

70. Use of an HSD17B13 specific inhibitor for the manufacture or preparation of a medicament for treating a liver disease or disorder.

71. Use of an HSD17B13 specific inhibitor for the treatment of a liver disease or disorder.

72. The use of claim 70 or 71, wherein the liver disease or disorder is fatty liver disease, chronic liver disease, liver cirrhosis, hepatic steatosis, steatohepatitis, nonalcoholic fatty liver disease (NAFLD), or nonalcoholic steatohepatitis (NASH).

73. The use of any of claims 70-72, wherein the compound reduces, improves, or regulates hepatic steatosis, liver fibrosis, triglyceride synthesis, lipid levels, hepatic lipids, ALT levels, NAFLD Activity Score (NAS), cholesterol levels, or triglyceride levels.

74. The use of any of claims 70-73, wherein the HSD17B13 specific inhibitor comprises a nucleic acid, a polypeptide, an antibody, or a small molecule.

75. The use of any of claims 70-74, wherein the HSD17B13 specific inhibitor comprises a modified oligonucleotide, wherein the modified oligonucleotide has a nucleobase sequence complementary to any one of SEQ ID NOs: 1-6.

76. The use of claim 75, wherein the compound is single-stranded.

77. The use of claim 75, wherein the compound is double-stranded

78. The use of any one of claims 75-77, wherein the modified oligonucleotide consists of 12 to 30 linked nucleosides.

79. The use of claim 78, wherein at least one of the nucleosides comprise a modified sugar moiety.

80. The use of claim 78 or claim 79, wherein at least one of the nucleosides comprise a modified nucleobase.

81. The use of any one of claims 78-80, wherein at least one internucleoside linkage of the modified oligonucleotide is a modified internucleoside linkage.

82. The method of claim 79, wherein the modified sugar is a bicyclic sugar or 2′-O-methyoxyethyl.

83. The method of claim 79, wherein the modified sugar comprises a 4′-CH(CH₃)—O-2′ bridge or a 4′-(CH₂)ₙ—O-2′ bridge, wherein n is 1 or 2.

84. The method of claim 80, wherein the modified nucleobase is a 5-methylcytosine.

85. The method of claim 81, wherein the at least one modified internucleoside linkage is a phosphorothioate internucleoside linkage.

86. The use of any one of claims 75-85, wherein the modified oligonucleotide has:
a gap segment consisting of linked deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar.


SEQUENCE LISTING (208 sequences)

SEQ ID NO: 1 - DNA, 1284 bp, Mus musculus
gcagcagcccataatacctgacagggctctctgtggagctctgagcagaggtgtctgttgtgagaacagagccatgaacctcatcctggaatttctccttctggtgggcgtcatcatctactcctacctggagtcactggtaaagttcttcattccccggagaaggaaatctgtgaccgggcagaccgttctcatcacgggggccggacacggaataggcaggctgactgcatatgaatttgcaaagcagaaaagcagactggttctatgggatatcaataagcgtggtgttgaggaaaccgcggacaaatgcaggaaactgggggccgtcgtgcacgtgtttgtggtggactgcagcaaccgggccgagatttacaactctgtggatcaggtaaagagagaagtaggtgatgtcgagatcgtggtaaacaacgccggggcgatatatccagcagaccttcttagtgccaaggacgaggagatcaccaagacctttgaggtcaatatcctcggacatttttggatcataaaagcactccttccatcgatgctgagaagaaactctggccacattgtcacagtggcttcggtgtgcggccatggagtgattccttatctcatcccttattgctccagcaagtttgctgctgtgggcttccaccgagcactgaccgcagaactggacaccttggggaaaaccggtatccaaacctcgtgtctctgccctgtgttcgtgaatactggcttcaccaaaaacccgagtacaaggttatggcctgtattagagccggaagaagttgcaaggagtctgatcaatggaatacttaccaacaagaaaatgatcttcgttccatcctatatcaatatttctctgatcctggaaaagtttcttcctgagcgtgccttaaaagcgatcagtcgtatacagaacattcaatttgaagcaattgtgggccacaaaaccaagatgaagtagtgcatgcagagacgtgtggacaccaatgatgtgaagccaagtttagaggggacacacagctttctttcacatgttttaagtgttccacatgtttaaaatgtaggcttgaccctagcagccatcgagtgcataagcgtggtcaactgtccttctagtttcctatacttatagatgtctcagctcctggtagtgggttctggcaatggatatgtaaaggagggaaaaggcaatccatgtgtttttataaaataaattactaaatagaaatgtgggcaaaagggcaagataataaagtcttgggcaaatgt

SEQ ID NO: 2 - DNA, 1730 bp, Mus musculus
gcagcagcccataatacctgacagggctctctgtggagctctgagcagaggtgtctgttgtgagaacagagccatgaacctcatcctggaatttctccttctggtgggcgtcatcatctactcctacctggagtcactggtaaagttcttcattccccggagaaggaaatctgtgaccgggcagaccgttctcatcacgggggccggacacggaataggcaggctgactgcatatgaatttgcaaagcagaaaagcagactggttctatgggatatcaataagcgtggtgttgaggaaaccgcggacaaatgcaggaaactgggggccgtcgtgcacgtgtttgtggtggactgcagcaaccgggccgagatttacaactctgtggatcaggtaaagagagaagtaggtgatgtcgagatcgtggtaaacaacgccggggcgatatatccagcagaccttcttagtgccaaggacgaggagatcaccaagacctttgaggtcaatatcctcggacatttttggatcataaaagcactccttccatcgatgctgagaagaaactctggccacattgtcacagtggcttcggtgtgcggccatggagtgattccttatctcatcccttattgctccagcaagtttgctgctgtgggcttccaccgagcactgaccgcagaactggacaccttggggaaaaccggtatccaaacctcgtgtctctgccctgtgttcgtgaatactggcttcaccaaaaacccgagtacaaggttatggcctgtattagagccggaagaagttgcaaggagtctgatcaatggaatacttaccaacaagaaaatgatcttcgttccatcctatatcaatatttctctgatcctggaaaaaggacctggattcagttccaagcacccacatggtggttctcaacagcctgttactccaatcccaggggatttgacaccctcttcagactttctcaaacactaggcactcgtggggtacgcatacgtcattcaggcaagccctcatacacataaaatagaataagcacatattttaaatgtgctactattactgatgttttaagccagttaaattatgtgattttcttaacagcaataaaatattgtagataatgtcatcatgctagctgcatcactctgactgtataaacccaagagaatttcactccatctccttctgccaaaatatttacaacacttaagaaataagggctgatttttattttttttaacataaaatatatgagacagttgtatcctgtcagctaaaaatgttttctctggcctgtgagttggctcagtagatgaagacacttgctgccaagcctaacaacctgaattcaactcccagaacccatgtaatgggaagacagatgggattcagagaaattgtcctctgatcattgtacccatgccttggtatatgagtatctgtgatggtttgtatatgctcagtccagggagtggcactactagaaggtgtggtcctgttggagtagatgtatcactgtgggtgtgggctttaataacctcatcctagctgcctggaagccagtattctgctagcagccttcatatgaagatacagaactctcagctgtgtttgcaccatgcctgcatgaacgctgccatgctcccaccttgatgataatggactgaacctctgaacctgtaagccagccccgattaaatgttgtcacttataa

SEQ ID NO: 3 - DNA, 28000 bp, Mus musculus
tcctgcatattttaaagatttttttatgatttttaattatgtgaacttgtgttcacatatgagtgggtacagatgcctaaagaggccagagaccttggatcaccctggcactggagtcagaggcaactgtgagctgccagacacgggtgctggcaactgaactcagtccctcagcaagaaccatataccctcttgacccctgagccatctctccggaccccacagcttttaatagtatctcaccaaactcatcttgtgaggagtaaataaatgtaagtgtgaaaagtccttaaatcagctcaccatgtgcttggtaaattcagaccattactatttgatctctataaccttgtactacttacaaattttcttttgtctgcatgcatgtgtgtgtgtgtgtgtgtgtgtgtgtgtgtgtgcacatgtgtgtgcgcgcatgtgtgtgtgcgcatgtgtgtgtgtatgctttttcagttagttcttctttgctttgttcataactgactttgctggggagctttgtgggtcaagatacagactctcattatgtaaacaggcttgtctggaaccttcaatactctggtctccacaccctcaatgctggaattacaggcaagtgccacacacccagcttcatacagctttggtttggttgtcggggtagtggctatctctatatatctgaataccttccgtaactcatataaatgaagtcagaacatgtgtggttcatctgactactgtagtgccaggaagcgttcatcaaggccaaaagaccaccaaggagccatttcaaatgtaatagcattagggtctccttattaaaatctctggcttgggccctcctcaaaccctcagcaggacaaggttttataggaaatggaagtaagtgagagggtgtctatcctggcaagcaactaactgaatgtctattgtagaccgacaggtgggtgctctgaagcaaaactgtatacagtcacaaatggtctgaaagtacatctgaaacaatctgactgatatttgattaactgttgctaggaagtatctaggagtaactctggccaagaacaagccgtggagtccatcctggtacatgggtccagcttgggttctgctgcaggtcaagttgtcaggcttttggtttttcctttaagatggaggttggtcctaagatggagtaggtttggcctctcactgtcttaggtgcctcatacaaacagattcagaacgtgggaggttttgtgtctggcttatttccttttgcatattataactgaggatcaccatgttgtagcatgtatctgaatttctttctttaaaggaagaatactatcccatcgtatgtatgtctgatattttacccatccattgatggacaagggtgatctctccttttccactattagaagttactctatccagaatgtgtccccttaatttcttatgctggaatctagctcctattaatatagaatgttggaactttgggaggtgattacgtcacgatggctgtctatggtttttataaagttgaccacagagaagttcctcctcattctttggggtgcagtggccatgaaatgacagctgtctgcaaaccaagatgccagttcccacttgacacacatacagcttaggttacacagacactaggactgtgtaataagccacctagcccacagtatttgtcaccaccgcccaggctgagacagtggcagcacttttaagttgacgtgtatgatatctggtctggtgattttcccagtgttatggcgagaactggctgtttcctataacctacattctcctccacctcaggggtttcagaggaagatcataactgtgaatgcatgcgttcaacagctaagctttgttgacccatattttagcttcctcatttttactttatatgcatgtgtgtgtgtgtgcatgtgtgtctgtgtgtatgtgagtgtgtgcatttgtgtatgtgtgtgcatgtgtgtgtctgtgtgtgtacatgtatatgtgtcatgtatatgtgtctatgcgtgtgtgtatgtgtgtgtatgtatctatgcatgtatgtttgtatgtgtgcatctgtatgtaagtgtgtctgtgtctgtgtgtgtacatgagtgtgtgtctatgtgtgtgtgtctccctctctgtgtgtatgtgtgtgtagagttggctatctccttccactatggatttggggatcatattcaggtaaaaggcttgcatggcaagcactttgactgctaagccatgtcactaatcccttaaagtttctttaatataatgtttctatttctaattcccccaaccctgatttaacatgaatgcacacatgtgcttgcttgtgttcgtgcatgcacacacacacacacacacacattcaaatgataacagaccattagctgatgcaatacttccagaatagctttgagaggtgaagtttcatgtcgaaacatggtgcataactgcttttcagataaccaggtccaaagggcacatcccactgggttccctgacctatgctgcgcttgtgttagagactggcatcatttcaaaaaccaaaggtgtatctgattcgtcaggggacacagtcaaagtctgcctcttctgcagcagcccataatacctgacagggctctctgtggagctctgagcagaggtgtctgttgtgagaacagagccatgaacctcatcctggaatttctccttctggtgggcgtcatcatctactcctacctggagtcactggtaaagttcttcattccccggagaaggaaatctgtgaccgggcagaccgttctcatcacgggggccggacacggaataggcaggctgactgcatatgaatttgcaaagcagaaaagcagactggttctatgggatatcaataaggtaataataggtgtttctaacaatcttttaaagtcacaaaataacaaggcatatgtttctctagagacgtaacttagtgctggcttttctctgtgttttcctttttcaaagttttaataaactcctgggtcctttctggatccctgcgacaggtgaacaagatggaaccaacgagctggagaactaagcctggggggtttcattttgtttgactggttggttggttggttggttggttctactgtggtgtacaacttgataacaagagacgcaggagccagagagcttcccagctgctgaaattaggcaagccacataacttacctcaacttcaaatttaaaaaacaaaaaacaaacaaaaaactctaccattgggataaaatctcagagggagacaaaatctacaggtaatgattgccagcaaccaaattggccaatatgcagtttattacagctaggggtgacttttatacttttagattttcatttattctgttattcatttgttcctttctgtgatagactgaagctgggtcttagtacctaccactgagccacagtaccttagcacgtgccaggcaagcactctatcaccgagcaaccgctcaggtttttacacatttgagggattgattctcaaaacaaacaaacaaaaccacatgaagaactaacagcagaaacccatggtacttccactcacactccagcaatcctaaaatacttacatctgaccttttgcagaagaagaaaaaatacttgccgggtcctgagcaagaagggtaaagaataatacttaagtgcttagcatggcctggtgcacgcagcatcacatatggaaatgtctacaccagtgcctggctttcactagtacatttaagaggttgttgatctctgtttatttataaatgctcttaactaccttaagtctgaaagtgttgacgtcctgtgtcactttctcctaaacatgcacattcccaaaaggtcgtgaaaaagatgtgccacaacaaactgtctctgggattgtaatctttgaagaactggactggggatgtagcccagcggtacaacaattatctagaatatacaaactagatattagatccagttgtggctgggtgtgagcctcagcactacaaggaaaccacaaaccgctgctgtcatgtaccggatacagtggctcagactagagagactggggttttggtgtttgtttgtttgtttgtttgtttgtttgttgagaagcagagtttccaaagctttccttggggttttgctcggcttcaggtaaccctctgttacgtttttgccatctgcagttaacaccacttttcccagccttcagaacgctcttgaacataaggagctaaatctgctttggttccttttgagcagtgaagaatcccgagaggcaccatgcatctttgcttagcagatacaggcgttggtgggcaagatgagctaacacggtacttggagatgattctgagattatcaaatcctgcacaggtcccaggcagtcttcttggagctgagaaaactgagtcagctgttctcagtccctcgtaaaaaatataaacgagaatgagaatacatcatgaagatggtccacgggtcttcagatcacctcccaagtgtgcatggtgggaagacatcaatacttagtagactacctttaatttgaaaattttcagacacagaaagagtaaaactagaaagaggtcacattaaagatgacttccactttttttttcctgtgcctgaaaactgagtaactcaaccttgtaaatttctctttgaattcttgaacttcatgatgaaacatttaagacatcaaatgattgactgaaaagctgcaagtgtaactccagtttccctgttcactgagaggccagctagctttccatcaaagaaatatggctccttttccttaaaaggcaagaagcaaggccacagagaagcctcctccacccccagctttaaaaacagtggcacagagatgtgaaacacaactgttaggaagggaattcagcagtggggagggaggggctgcaggaaggtaacaggggataaaataataagagcatacatgtgtatgcaaggaatgaaaagcaaatttaagaagaacgcaatgacacaatagcaaaactgtggacccagccaaggtgcccacccagagacatgggttggaaaatgtggtttcacacacaggacatcgttcagtcttttggaagaatgacatcattctgtttgctggacagtggaaaaaactggcagtcacaactttaggtgaaaagggagccagactcacaaagggaaggatcagtggggggcggggggggggggaggtcactctgctctggtcccagcattggaccaggaaccaaggcaagagggttgctgcaagttagaacccagccctggctacagatgaaattatagcaagagacaaaagagggtaggggaaggcagcaagcatgggctctgtcatgcctcgtgtgtgggaatgtcatcatggaacatactagtttgtacgttaataaaaaggacacagggattagcaatgggcagaggggttgacaccccagaaggtgtttcttgtgcaaaagtgacgaagagatacattcttccatgctccagattaattataaggctggatagttcgcaaattattagcatctatcactcatacatgatgctaggtttcaccgtaagctttcatagatgtgtatattctggtcatattcactgttcacccatcattatctctggtcccctcccactcttgctgaccccctttctaagtgcccccttgtgctttcatgtgtgtgtgctaatgactttagataggattgctaacttgagcctagacgagggcttgttacaggagtagggatggcttgctggtggcgaagaaagtaaccctgatacactgtttcttcagtcacatcttttgcttatgctactgaagaaagtaacactgataaattgtttcttcttcagtcaaatctttgcttgaggctctggttactgcctctctctacagctctgacgtctgtatgaggctagtgaggagggcagctgggccatctgacagtaagcagccttcattagtggaaataaagccaacattagtggaaaactctcctacccagaccacagctctattccacagccccacgaggacctatgctctatgcggcagtccatcccaagtctatgtgaagactatgttggtttcgtaagtgacaccttaaccctccttaaagtctgttctttaagacttctattgttgctttgtggtttagccaagataaggaaatgcaaagggtcggaggggaggcaagcagtgtaagccaaacataccatccactcaggcaaggcccaaatgatggctacactattgccatcccagcttgcacaagaaagagcttcctgggtgtcctcacagagaagtggggacaaccatcagaggaaaaagtgtgatttttaaaaagaatttgaaccccataaaatcaataggtgtcaaaacttggaaagagtccaaattttcctgaaatgtaggttaaaaaaaaaaaaaaagccaagggtagtgactcacacctgtaagtgcttgaggaggttgaggcaggaggattggcctgagttgaagacccgcttagggttcacatagtaagcaagggatagagaatgacacctggtctaaaacaccaagagagaacacacagacacacagacacacacacacacaccacattaggacaatcttaggtaagaggcttctggatttaggctaaaaattagctgtctccatgtcttggaggacttgtgtgcaattctcactttcgtgagaattcagctgttggctcttggcctttttccctccagagcttcagaagaataaatactcagttaagactgtcacgtgtagtaagttacagtttcctcgtgcaagcttttcctggataagattttctcctcgcatccgcgggggtgaatccacgttgtgataagccatctagtgtcactctagagggctgcttagcactctactgccaacaatgggtgatgcccggcaactgtgtggactagtcttccttctgtcctccgtgctcagacatcttccctgacactgtgaggagcagcatcttcgataacttataggaagcccttcatgctaccacagcatgacgctagacaaatcacctacaggcactcccaggacaagatgcgaagctgcaaaagccttaacccgaagggcttgtttctctttgattccgctgctgctttggccagctaattagttaatgtagttagatgagttcacataacgaggcttattggacaaggatccgaaagtattttacagagcctgatcttttctcaaatttgcctcccccctccttcacccctcccgccccccaccccgcccagcgtggtgttgaggaaaccgcggacaaatgcaggaaactgggggccgtcgtgcacgtgtttgtggtggactgcagcaaccgggccgagatttacaactctgtggatcaggtgagaacccgggtgcaggtgtcgcatcagctgacactgagctgggaaacgaaaaggtgggctagggaaggttgacttccggtagacggcagccgccctgtcaagttgctgtgtgaccgaaggagtgaatattaatatccactcagctctggcctctgtcactgtttcatattctgagtcaaatctgctgtgtctaaaaagtttggcccagaggaaacgaaagatgatttatcaagagaaaatttgatctcttttctaacttttttaaaattagactttgttctgctttttctttaacttattccctctgagattttattggtttgcgtataatctagtcttttatgaaaaacagagttaatgactttttaaacattgtatttgttttatttactgcatcgtgtgtgtgtgtgtgtgtgtgtgtgtgtgtgtgtgtgtgtgtgcgcgcgcgcatggtgggggtgtatgtagaatttagatgacaaattttgaaagctggtctctccttccatgatggggattctgggggttgttaggtatggcaaaaacagaagaggaggaggaggaggaggaggaggaggaggaggaggaggtggtggtggtggtggtggtggtgaagaagaagaaggagcaggaggaggggaggaggggaggagtaagaggagaaagaggaaagggaaggggaggggggagggggagaagaaaatgcctttacccactgagccgtctccccaccccacaatacctactctttcaaaaagtgaacatcaagagcttaccaaaaaaaaaaaaacaaaaaacaaaaaacaaaaaaacaaaaaacaaaccctccgctaagctactgccttctttaatctgatttaatttttaaaaatcccattagcctcactatagataagcaaatagaagcttggaaggtttggcccagacaaagaccacgtggccagtgggtagcaaaaccaagattaattacttaacactttaaaaatatcttaacagaacttaagtattatttaccagcacaaacattaattcttgtttatttaagttcataagttttcaccctacctatcacatactgagcacagaatttagggttttaaccaaaacaggttagccctacctcatgacaggtttcccttcctgtcactgcttgggctggttctttcacgcctgaactctgagacctcgtgcactggaataacaaaagcctagatggcatagtgaccaagctagctgtcaccaaagccagtgagcccagacagaaatgcatcgtaaagctattctttattcttggaagtcagatcgatatcttggatatctgtcttccatgtcacgctgtgatgtctgtgtgtctctcattatcatcccttggacaacttcctccttttgtgttattgggctcaggcatttcttggagttgagttggcctagtttatgctggtatcaatcttagactcttgggatggctaggggtagggtgtggagtggttagctcagaacacatggtgtggagttgctgagctataaatattgtgtatgctctggtttctttttacatggtggagtatatatatccttataatatagaacataaaaatgcctctacccatctgtctcctatcaacatgagctttgtgagaatgaccggtcctgtgtgtgcattttaaaaggtaaagagagaagtaggtgatgtcgagatcgtggtaaacaacgccggggcgatatatccagcagaccttcttagtgccaaggacgaggagatcaccaagacctttgaggtcaatatcctcggacatttttgggtaagtcagaaacatttctgggtgtgtgtgtctgacatctcttcggtacaggaaactcattgaggtcacaatatttgtggagggaggtgtttttggttttgtttcttaagatttatttatttatttattttatgtatgtgagtacactgtagctgtcttcagacacaccagaagagggcatccgatcccgttacagatggttgtgagccaccatgaggttgttgggaattgaactcagtacctctgggagtgctattaaccactgagccatctctccagccttgttttttgttttgcaagtcaggaatcttataatgtgtttgaatagaagagccacaggtctctggcaacagggttcagagttcaaggagacacaggacacccaaaagagagagccatttggtttattttgtcaaacaaaatctaacatagggcctgatgaataccagatctacaaaaagtaaattaataataaaaaatttattaattaaaaatatttgtaaaggtttactgatatttcctctgtacatcttttcactattttagaggaaatgtatgctgcatacattggtctgcatcttcctttcataggtgtttagagctgaaaggacatagaatacttagatttgtgcagaagttggggacacttagagttaggctctgagataagtagacattttgacataacattattatctacaaagttcctgcctgagaacttcacaattgagttaccaataggggggggaaacaatctcaaaatgttttactgtagctcacaaatttgcattgatctatattcccagctaatctcagtcataagcagcctatgggcggtgggatgagcatgcctattagttctggagtgtagagagaatgaatccaattccttactattgggagtttgtgagaaactgaagtacccttctcagccctttggagggagcatttgtatgaattaataccgtgggacctcaaaattactatagcacctggagcaaacctggatttgaagaacatccttcatcctcatgacccagctaacatgacagtcacacaacacagggaccctatatactagttatctaataattaataggaaattataactatgtcacaaaatatatcaaaaggtagaggtttagagaaccaggagccctcattacctccatacagatctgcaggcagggctgatagggggattatcttggcaacctgggctctaggaatttacacagaatttgaagagtgggtctgtaaaattctcataaaatgttctgatatttcccactgtacagtgaggcttttgtttcccataatgtatcctatactgtatactgttacaagagagagatttgggtctgctccctgtgcacggtggttgtgggccaggatagagcttccaccgctttgacgtgagtaaagatccccattcttcaaattggagccaagaacttgtaaaacggtaaaccatcctccagttcccgcactggtggatctctcccttgacaatattgcttagggctcagcagcaccaggcttgcctggcatgcacaaagtcctctggttaattcctagcacggcggaaaagtaacaatgttgcttctgtggcaaatcccggaaggagctacttaattcttcctggagggatgaaggggcatgctcgcccgctagcactggtcctcagcacaatctaggggactcagctctgagtgctgtatgcacgcttgcacaggtcctctgcatgatctaagggacactctgagtactgcctggaaccatcacagttactctctctttctgtcactgtgtggaaggaacctcagggtcttacaagtgtgtaatttccaatgcctcctcctccgctatggtggaggcattggaaatgaaagagcctggcctggaatccacctaataaccgtgtcattgtttctgttctttttacgtggaccttaagatcataaaagcactccttccatcgatgctgagaagaaactctggccacattgtcacagtggcttcggtgtgcggccatggagtgattccttatctcatcccttattggtaggtgtctcttgctcacagcgctgtgcatttctgcttattagtttggggattatttatagggattattataattagtatatttatgtgttttataaatattatatactataattttataaatgtattcatatattatataactaaataattatagggattaattataggaaattattagggattattagttttcaggcctcaatgcccacagcctagtaaagtgcccagaagctaacacaagcacagaggtatagcgtggtctatgtgtgtgcatgtgtgagagaagagaacggagcagccctgccttcccaggagatgcacacactgcacaccatgtcccagggacccagacacactcgcagtctgcagcatatccctgagaagtctgctattgccatcttgtttccttactgctatacgatgatctcagaaacacttttaccaagatctgtggatgtagcagatgcaggtgttatttctttataggcatattcaaagctaaacttgaccatggactaagcaatctctcacactatacatggtcacagatgagttaaatttcagtgtagttcaggtttccattacacgcacacactcacacacatacacatacacacacaatacacacacatgcacacacatacaccacacacaccatacacaacacatacacacacaacacacattcacatatgcacacacatatacacacacacacatcacacatgcacacaggagaggaagacagagacagcaataacttctaacactgatattttttgtcacctggacaaaaactacaattaaaacttatatagctccatatatgcatatgctaatttattgaattctccaagacagccttatattttgttatctatattttataatcagaaaaatgaaagaacaaaaggttaaaggttgcacatgcaaacacaaccctaccaatgccatctggagcccctttgctccaaatactcgttgtgacattaggacacacatgagactgccagtgtcaagggagaaaacatcagctctttaatcaagagaagagaaatgagctcagacccacaagtttgtcgatgttggtttcacagtgttttaagtgtgtttttctgactcacatttttggagataaaaactaataaaacctgattaagaagcacttggaaggttgcctggaccatagagaagggtcacacatacagtgagaaacattaagcctatctgtaaagtggtccttttgctatctttgctagttagaagttagaaacaggggctttggggttagcaatggttgaagacatgcatgtgtcaatccctgagttcaatccccagcacctaaaagaaaaaaggaaaaagaaacaggtattttgcatgctcagaattctgcttcctacataagaaaataaactcgagcggcaggtgacaggtctggacacatgaagctgccttctctcaggcagctgagactgtccccaccaccaccaccaccaccaccaccaccacacacacacacacacacacacacacacacacacacacacacacacacacacctttgctgatggctgattttctcacagctccagcaagtttgctgctgtgggcttccaccgagcactgaccgcagaactggacaccttggggaaaaccggtatccaaacctcgtgtctctgccctgtgttcgtgaatactggcttcaccaaaaacccgagtacaaggtgagatggaagacctttaagcgggagcccccccctccaggcaatggagtcttgagtaggtcataggtacttaacaagcatgtcacgatcatttaaataagtccagtagcatgatctcaacccctaggctgacactgaggtgcccatgtcagatgtggctctaaggctggattgaagactccattgcaaaattaggaaggggagagacagaggcaggataatctaaaaagaattgatttgtgctgctgtgtgatggtttgctagtccacatcacaggcatcacaatcgcacgggagtctgcggaaaccacagactatctcatgggattcagttccaacacccacacagccttgcctcggcatgctaaccttggtgtgacatctaaaatccatttttatgctttgatttttccctcctcttcattaatgggcaaggctccaacactggacatgggttatacgcatttctcagttatacacgggtgcaacttttgaatcacgaaacgccccagtgctgtaacagaacactccttgtacaaatcttaaattcccataaaattgcaggagaagcaaatcttagtacgagatgcctcagaacaactcagctctgtggatacccacacacaacgggaagtgtactcagacatagaatattctttttacatacagcaggcattggagatccggtgtttccttcctggccattttgtcccctggctcctcagtccctccccatcttgtaccttgttgtgatttaattattttgacctggtgaggtaacagacagacctactggacccaagagtacaagaacatgataacaagttatcagtgtgggatgtgggaaggggagagggtgatgttcactcagggaagcctggaacattgtaaatcctaattctaaatttggtgaagagacaggataatttccttcatttgttaattaaaaacaatataaatcaagaggctggggttttacctcaacatcagggtatgtgtgaaagttctggatttaattccagcatccaaaggaagaaataaaatataaacggagataaactctttccccctttccttcaggttatggcctgtattagagccggaagaagttgcaaggagtctgatcaatggaatacttaccaacaagaaaatgatcttcgttccatcctatatcaatatttctctgatcctggaaaagtaagttcctcgcagaaccctgaaacactcaaacacaaatgttagaaatgttgctatggaaactccccatggctgcagtgaaagtcacgttttccatgttgccttacccatgagtctttgattccctgataccctttctggaatacatctaccatcttggcccaaattttgttcaagtctatttaacagaaagaaagaaaaactacagggtatgggcacaccataaaggctctgtgtgtgtgttcatgtggtgtatgtgggggatagagatgtggtacttgtggagacagaggcaaagctctcaggaaccatccacctaggtttctgatttgggtttatttctcttctctccttcccctcccctcccctcccctcccttcccctcccttcccctcccctcccctcctctcttccttttcctttcctttcctttccttttcttttcttttcttctcttctcttctcttctcttctcttctcttctcttctcttctcttctcttcctcttccttccctccctccctccctctctttcctctctctctttttttctgatacaatctcttattgagacctggactcaccagcttggctaggatagatatgcatcaccagggatatcccaggccctctattcccacagtactggggttacaagtgctgacagctatgcccagctgtgtgtgtgttctgaggattgaactcaggccctcctgttgtcgtggcaatctccctaaaccctcgggtgctcagttttgcagttctttgtgacagtgtgcatctcgaatgtctcataacatgctttcacatgtttctatttcacttcatttataacccaagagccttatgatagaatcacttcttttcatctcaggcttgtgagcccccaaagagggcaaaatcccacaaattgaatttcctcaacacagctcaaaacaggtctcattaaaagttgaaaaaaaattccaaaattggcatgtatatgaccaaagtgcaaaaatggttgactgctctgcctgggataggaccagttacctgtctcaaagcccttgccacactgtgggtgatttgttacgctggcaacagggatttttgttgctccttgcatttaactccttttggagagagacagttctgtgatcagaagcaatcactagtctctgggcttgttgttcaatggcctgagcaagctctctccttaccacctcacagcagggtccccacctcccagtctcttctcccaccttctgccctctatccagggaagtaaatgttgctttagtaaacaataccaatgtgatctgaaatttggctttctctgtagaaatcgcacctcctgcctctttcacttcactcacacccttccctattctgtcaactcagacctaaccctccgacatgctgagaggtattcagcccctgtaaacccttccttccacatcttgttatagtcagctgatggttttctgtattcatttccagttggccatgtttaaccgctgggcagcgcaatacactaagaggcagtagcttttgaaaaggctctttagtttttctttctctaaaataataggtctggacacaggcagaggggggaagggggtgtatgtggtggactctcagttcttggccccagaatacactgccgttttttggaagtgagcttggtgtcggttttaggttgtcattggtagaagacgtcatccaattttgctgttctgagagaacaacgctatattatatatagataactgtagatggggtgggggaggggagactaaacaataagcaaagagtagaggaaaactggacttgagtaagtatgctcttggcgctttttaaattgacaacaaaaaggaacactggcatcaaaacttcaaactgaaatttctcattttatttttaatgtaggtttcttcctgagcgtgccttaaaagcgatcagtcgtatacagaacattcaatttgaagcaattgtgggccacaaaaccaagatgaagtagtgcatgcagagacgtgtggacaccaatgatgtgaagccaagtttagaggggacacacagctttctttcacatgttttaagtgttccacatgtttaaaatgtaggcttgaccctagcagccatcgagtgcataagcgtggtcaactgtccttctagtttcctatacttatagatgtctcagctcctggtagtgggttctggcaatggatatgtaaaggagggaaaaggcaatccatgtgtttttataaaataaattactaaatagaaatgtgggcaaaagggcaagataataaagtcttgggcaaatgttggcacgctgtagtgatttctctttcgatggagagattggtggtgttttaaatttgctctttgtggacaggatgaaaactctacctggtttccttgacatatgaattaggaaggagtatttttgttccacattttttttctcagtgtgaggttttcttcacatcatgtgaatggacaatgaggtcattttatttacctaacttggggaagcaaatattgacacagatttgcaactgtgaaatctcaacaccttgttctttgctttagaaagaccttggacacatttgtttttctttttattctattaggcaataactgaaaatacccaaccgtatacatagtaacagtgacatgacctcccacacagatcccattatgaagcaggaaaccttcctgggttttcctttggcttctgtattttgctgcaggggactgatcccacagactaagacaggttacataggcagtctgtgttgacctcctgtctatcaacttgcagagttacttccacattttgctttgatacctttaaagtgaagctcagagggatgcctcagtatgtgacaatgatctcttttgcccacgtctgcatattttgagtgtaaatatcttccctacaaggtccagctccatttctaaatatttttctatgataatcttcacagtcacagttctcactcctgctaaccattgcagccgcttaggctaccccaggaagcacagatccctaagtaagaccccatctacacaaatcatttagttagagacacatcatagaattcctaggtccctgcgagcacggggtcatgatttattgatgagtatttcagaatggtggaagtatttgtttattgtctactcagttcatggaaacaacctttttggtaagcagacagtatctcctcttttttttaatggtagaatatgatattgcatatacataagcacattttgaacatagctgaatataagtatcactgtttcatccttcttcttcttcttcttcttcttcttcttcttcttcttcttcttcttcttcttccggagcatataattttattgctcttcttttttaaagaatagaagaatatcttttatactaggtccatgggctatctagtgtctggtttttgttcacccaagcagtgctagatacaagctccatcttgtgtagtaggctttaagtcaaaacagatttggttggttactctcacaacattgggctaccattttcctaccatatcttttaggcacaacaccaatgttgatccatgggtgtgtggctttgtttgttgttcttttcctaggagcatgcagaatgacttcctacacctaagatactagtagataggagtgaagagtctatgtagtcaccagattgacttctagatgttcagtgagttgagttgcctttagcaataggaccttgttgacaggtggtgtagagaaacatatggccatggtaacagcttgagttctttgcagattcccatgtaaacatttggacaacatctcagtaaaatgtaattgagtttgaatactggaagcttcttttggtgaggaaagataccctgttagaaacaagtctcctttgttatagggtgatttcatttatatgtgtaattgcctttgtatgtgtatatttttctatcaatctgtctatgtatcatctatctaatctctctgtcatctaatatatgtatcatctgtcagttatccacctatcacacatatatagctattgtctatcaatcaccttttaatctaccttttcattctatcaaactattaatatatcaatcacttatgtatttgtgattgatctatcttttaaccatctaattatcacatatctttcttctattgatgatctattttcatctgtttctctgtctgcctctcatttccttttgaatagctctagctatcatatgcaaagggaagacattttcacaaaaataagcagtcttgagggtcctcatgatgttttcagtccctcagtgtctcattccaaaagcccaaagacaccatagaattcagacagaatgccatgtaacaaagccccacactcaatacccagttacaaccaccttagcagaacactaaagagattaaactatccttggctgccactaaaatcaggtagtcttcatggtaagtgtggatctagcttttggggaagtcctgtattctttgctaacccactttcatttcaagattgaacaaaacacaggaagatggtgccctgtgaggccttgacactcacatagcctcagtccagttagctccctgtccatgtgttcacaacagatgcttgtgataaagaaagccccacaggatgggtgaggtctgagtttctgaccacagatccttcctgttatttttgatgttatttttatgtttgtgtggttatcttcttttgggtttgttgaaagaggatttctttttctttttctaaggtgtagtttcccttcttgtgttggcattttccatctattatcctttatagggctggatttgtggaaagatattgtgtaaatttggtttgtcatggaatatcttggtctctctgtctatggtaattgagagttttgctgggtatagtagcctaggctggcatttgtgttctcttaggatctgtatgacatctgcctaggatcttctagctttcatagtctctggtaagaagtctggtggaattctgataggtctgccttttacttgaccttttccccttactgcttttaatattctttctttgttttgtgcatttgatgttttgattattatgtgacaggaggaatttcttttctggtcaagtctctttggagttctgtaggcttcttgtatgtttatgggcatctctttctttagattagggaagttttcttctataactttgttaaagatatttactggccctttaagttggggatcttcactctcttctatacctattatccttaggtttggtcttctcattgtgtcctggatttccaggatgttttgggttaggagctttttgctttttgcattttctttgactgttgtgtcaatgttttctatggtatcttctgcacctgagattctctcttctacctcttgtatactgctggtgatgcttgcatctataactactgatctctttcctaggttttctatctccagggttgtccctttgtgatttctttattgtttctatttccattttcagatcctggatagttttgttcaactccttcacctatttggttgtgttttcctgtaattctttaagggagttttgtgtttcctctttaagggtttctagacatttacctgtgtttctttaaaggagttatttatgtccttaaagtcctctatcatcatcatgagatgtgattttaactcagggtcttgcttttctggtgtgttgggatatccagggctcactgtgttgggagaactgggttctgatgatgccaagtagccttggtttctgttgcttatgttcttgctcttacctcttgccacctggttatctctggtgttaactggtctggctgtctctgactgtggcttgtcccttctgcaagtctgcattgttagtacttctgggagaccagttctctcaggaggaatttgggtatggagatctgtggcacagggtcagctctggagtgtagacagaaaccagaaggatcctgtgcctggttgttccttgattcctgtgtcctgatggctctgggaaggtttcgcttgggccacgagtttgaacagaagtggtgctcttacctgtgctcgggggtgtgtcagcactcctggcaaaccagctctctcctggccgtatttgggtatgtagcggtgtggcacaggatcagctccgggcacagacgcaaaccagaaagcttctgtcccaggccgctcctggttcctgtgtcctgagagttccaggagggtccttctgagcagcagtggtggttttacctgagttcaaaggcttgtccacactcctgggaggcaagctctctcccagcactatttgggtatggagccctgtggcagaggatcagctctgggccaaacataaaccggaaggctgacatctttcaaaaggagtctttttttctttctttttttttgggggggggggggttgatttttttttttttcgagacagggtgtctctgtatagccctggctgtcctggaactcactctgtagaccaggctggtctcgaactcagaaatctgcctgcctctgcctcccgcatgctgggattaaaggtgtgcgccaccacgacccgctcaaaaggacttaatgttctttatttaacaatgttatatattttctagcaacctatcttctctccctctctccctttctgcttctctctttcttcctctttctttttctgcacctttctctctctgcctctctctcttcctttctctctgtctctgtctctctctctctctctttctctctctctctctctctctctctctctctctcgtgtgtgtgtgtgtgtgtgtgtgtgtgtgtgtgtgtgtgtgtgtgtattaaaggctgcattacaccaggcaaaggctgtgttacaccaggtaaaggctacattacacaggtaaaggttgtgttatgccaagcaaaggagtttctttgaactttaatctcattgcacccacaagagagtaagcatacctcaggctaggtgtgttcactggaatacgtgaagtaaaggttgaaacttctgtttatgactaattctttatcttaaaactttgttttttacagtatatacgatatgtgattatattatgacacgatatatactaagtaaatattaggtacacactgaagtttagctgcaatgctcagtcagacccacagtcctttgcccactcctgttctctctctctctctctccctctctgtctctctctctctctcgctctgtactctatagctgtctttgagcaaagatcacaggaaccaaaaaactgagaggtccagagcatgagcctagtgcacattttagtattcctacatggttataggtgcactgtctcctgtccatggtgtctaccaccctaggaaaagctgcagggtctagagatcttagaaatagaataagccaggctaaatacacagaccactgggtaaatcagtgcagtggtcactgttgtgggctagtcaccagagaagactgttcaagccaatggaaagtcattactagctgactggtgactacactggatgttcaggatcccagtgtagcactgagccttcctcaggttgagcttttaagcacaaaaaccatgttcttggttgacgtacttcagttaacaagaacagttagctggaagaggaactacagaagctaaaaggcaacattagtacattatgagactttaccaaaacaatgaaccctaatgaattatgccttttagttttgacaggtagggctgtctaagtgctgagttttacagcctgaacagtacttccatcatggagtcagatatgctaagatctgggaccctgttacattccacactgttcttagtgaatgagtcaaatcatggattcatcctgttttagtttaagatgaagaatgagaattaaagtgctgacacaaaatcattagccagggagaccacgagaatggagactggtactgactacttcctcaaccatgtctatattttctctaattattccttaccaattagtatagaaacaataggtttctttcagagccatgtatagtttcctttatgtcatttgaagtctaggcctctccaattttgtagggagaatctacctgcaagggaatctaactgttgttttaattcagaaactgaaacttcaaatacctgtaggtcctgatcaacctatcctcttagtttggaaagcctcagaagaggcatatttagtcaaaccattaatatatgctcaccaagtagtctcagaggtttggtaatattgctgttgtccagatgaattaaaccatcgggcagaataacacaccttggaatgtggggaagtgcgtaggttaaaaattttggatattagacaggttttagccccttctaagtcctctagcattaatctgggctgcccccagtcacagtgagttttgtcagtcaatagattggttagtccccatttctctgtcctacagcagatgggtgtctaagcagtttcatcaaccctggatggttttgactgggcatgattacctctgcctcccagggactctttctgtggcatagagtctgatgctccaggcctttttggttttacaaagaccagaatcttttaaagagttttgggggcctagtgagatggttaagaacactgactgttcttccaaaggtcctgagttcaattcccagcgaccacatggtgggtcacaaccatccgtaatgagatctgacaccctcttctggtgtgtctgaagacagctacagtgtagtgtacttagatataataataaataaatctaaaagaattttctgtaggaagcttcggttttaagttccaatttttggaatggaaactcctctgttttaagctgtggtacagggtcagctctggggttcagacagaaaccagaaggatcctgtccccaactgttctttggttccagtgtcctgatggctctgggcatgtccctcttgggccaggaatttgagcagaaatggtggtcttacctgtccacactcacaggcatgtccacactcctgggagaccagttctctcctggtggtatttgggtatggagcgctgtggcacaggatcaagctctgggtacaggcggccagtatctcctctcgtttgatcctgaattgttaaattctttattatcaaataaaccattcttggccaatcttgtggccacactgaatgccagtagtaaggagcctatgaacatctgacatcactgtgcaacaataaaagacggcaccatgttcagacataaccaatggaattggattcatagatgtacacatgtggagtgaagatcaatagtccagagccagaagaggaccagaggccccagcaacattgctcccagacctgctaacactttggtttcctccatacactgctgaacaagagagtacttactccagaaagctatgagctgggtgatgaggagagggctcaggttttaaaagcacttctctttctttcagaggacctggattcagttccaagcacccacatggtggttctcaacagcctgttactccaatcccaggggatttgacaccctcttcagactttctcaaacactaggcactcgtggggtacgcatacgtcattcaggcaagccctcatacacataaaatagaataagcacatattttaaatgtgctactattactgatgttttaagccagttaaattatgtgattttcttaacagcaataaaatattgtagataatgtcatcatgctagctgcatcactctgactgtataaacccaagagaatttcactccatctccttctgccaaaatatttacaacacttaagaaataagggctgatttttattttttttaacataaaatatatgagacagttgtatcctgtcagctaaaaatgttttctctggcctgtgagttggctcagtagatgaagacacttgctgccaagcctaacaacctgaattcaactcccagaacccatgtaatgggaagacagatgggattcagagaaattgtcctctgatcattgtacccatgccttggtatatgagtatctgtgatggtttgtatatgctcagtccagggagtggcactactagaaggtgtggtcctgttggagtagatgtatcactgtgggtgtgggctttaataacctcatcctagctgcctggaagccagtattctgctagcagccttcatatgaagatacagaactctcagctgtgtttgcaccatgcctgcatgaacgctgccatgctcccaccttgatgataatggactgaacctctgaacctgtaagccagccccgattaaatgttgtcacttataagacgtgccttgctcatggtgtctgttcacagcagcaaaactctaactaaaatagcatatctctctcactctctctctctctctctctctctctctctctctgtgtgtgtgtgtgtgtgtgtgtgtgtgtgtgtgtgtgtgtgtgtgtgtgaagtggaaacttaagggttctttagaaaatcagtggcgttgggtggtgttttgctggggcaaacacatgaagggacattttcctgagttggacacaggcgtaaagttaaggcagactggtgaaagactgattcactggagcagacacaggagaaacgatgttctgctaaagcaagcacatggaagggcatgtgatgaaggactctttgctaacaaaacacttgtattggtctgacttactttgcatagttgagctccatttgttgggactccttagagagaaatgcataaaaaaaaaaaaaacttctggtggtgtgctgtagtctcttgctacttctgtggactcgagctgattggcagagtgatgtcatctgagacagatgcacgtgcggaggcaagacccatggaggacacctgatgtttggaaggagtataaatagaactcaacagatggtgatgggggctgagcttggcttgcttatagagctagcttgtgcaacacttgttggtctcttgtctttgctggtcttcatttcactgagagagcacagacgagaactttgtctggtgctcctcccttgattcctcctactgacttgtgccaaggctgaggcctggctgtctctgctaggtcgtgccaccttgttgctaacccgactctgcccaactgaactgctagtgtatcggtgaagtgtttgtgagtagattgagctgccattgctaacctgtgacctgaactgcagatttccagacaatacagatagtagctgctccaaagaaccatttctaaataggtctatcttccccatgtcctttctttcccactactcctggtgggtagtgagctaagagggaagttaaagcatttaagaaccatcattaaaaataggttttgaaaaaaattaaagttgcgcattgtgaaatgctaagaaaatgttttggtttgctttccttgagtcagaatcctatctgttgccctatactgagtactaactcaatatgtaatccaaatggattttgagacagaatcctgtctcagctcccaagtgctgattacagatgtgtgccacagaacccaactgaaaggaaacacttataagtattttataaatatctaagttgtttaagaggaatatttatacccacagatgtttttctccataagaaaatattactatatgttgattgagatattactacagttgatcaattcccccaaaagctgaaatcttaaagataggtttccccatgacctagaatatgtatttaattttggtttctacttggccatgcaggaagagcaggaggagtgtggagagaagccattggcacgcccggtgactttgtttctctgctgatgtgtaaggatctggaaaagctagccaccagacctcaggccacactggagcagatggctcagcactgcttggcattgatgtcaccctctgcaaggcttgcttcatacactttctgctgggaataaaatgaaacctagatacacttcagttcacatttggaacgtgactgcggacaatcacttgtaatgcaaaacaccccaaagccttttccccaattttatcaactctgcctcctacacacatattttatttgttctgaaagacttaatccactctgaaggaagcatgacactcccttgggctaggctgatgaagcaccacacggcttggctggctcagagtcaaggtaaactgtgctgattgtgggcaactgtgagaaacagatgacacatctgtgttttcccatgaggttagaatttgctgaatgtccgtaaattcacaaaccaccttcattggcagcaatttgccaaggagttccattttcttcccttgatagccagccaagctcttaaatacaaagaaccctcagatcacgcaacgccagccactgtgcaagtatgggggtggggagagagagtgtgtatgcaggttacatcatggccacaaaaggctaaaggatgctctaccagtgagctcttaacaagggttgagaggcagacgaggaagctggaagggttgccaggggctctctgtgggagaagagattggttggaggtactgtcatggaacccagggtatgacaccagagttgtactgagcttcggggttgatttgagctgaagaagctagttggagaagcatggggctgtagaggaaaggaagggatctaggttcagatggccacatgaggcgatggatagcaagcccagatggacaagtgatagatggacaaacggatggcaataggctatatctgcaatggagacaaagttcctgtgatagctgggagtcctctgcctgttgttggcatacacacagggtaatcagatgacggatgccagggccattgtcattgtgacatcaggggttctgttttccaggtacaatgttgcatcctgtctttgggctgttgtgtctgataagttcttaggcttggttttcatgacaggattttcatatacacacgtgcttacaaaataccaatttgctgtgatgttcctagaatggaatcttatgttttggtttgtttttttacttttaggaacgagtcatcccccgccacctccacacacgccttatgagcagagggggttatgtgcccagattgagttatgtgcccaccaaagcgcagagtcatcctgcttctgtgcactcaaaatgggtatcaaaatgtttgtaaagtggagttcctcggcccaagacacagcctgggaaaaccagggttttgaacacatagagccaggcatggcctgatctcaactttcagcagtgacacactccttgagtgaggaaatgaggcagctcagggtggtaagtttcacgcctgttatccctccacatcccctgcatgtcattccgaagtcctgcttgccgatggcttgcagatccaggaggaaaccaagctttcttgacagagaaaaataggctctaaccccatcctgtgggttgcatcctcacaacccaattacttcccatgtgctcagcctcctagcggcatctcactgagagataagatctcaatatatgaatttagacaaacaataacaatgaatcaattttttaaaaataccaatttatcttttcttagctagatggaaagatctttctattcaagggctaacactggataatcttggagatgtgtcctatctgttgctgttgtgcctgggaaatgttaatgattccccaaaacatacacaagagccaatctgatgcaactcatagcagggtctttattctatttgagctagctcgccccccccccatgcaccacc

SEQ ID NO: 4 - DNA, 2289 bp, Homo sapiens
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 5 - DNA, 2397 bp, Homo sapiens
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 6 - DNA, 25000 bp, Homo sapiens
aggaggctgaggcacaagaatcacttgaacctgggaggcggaggttgcagcgagctgagattgtaccactgcactccagcctgggcgatagagcgagactccgtctcaaaaacaaacaaaacaaaacaacaacaacaacaacaacaaaatcaagggacaaagtgtatgtttctagcattgttggggcaaatatgaaccctgtaattgaggaacatgaacattccgtcccatagaaaacaagcaaaaagaaaaaaaaagagagagagctcactaggttgtctcatatgaaataaaatttctcagcaaccaaactatttgagaacaatgttaagaagtgttgttcactgtctcaaagattcttaattcttgcataatacaaatatccagtgggtaaaataatctagattccactgtaaattttaaaactgcctttcaaaactgcatcagtactgtattgatttagatagcattggtctggccccctctgagcaagctggatattcaaatatagacattcaatctgaaaacagattcctgcactgcagcccagcatgaggtctgatctaaagcacctatgtcaatatttaacagctccatatacatgtcacagcaaacctgcatgcagaaagatataaccaagtactatttttttgccataaatttatccaaggttcaaactgaaagaaccaattacactgtctccaatataatataagtataggccaggtctcaaccttagttgagggtactcacgtttgggctaaataattcttggtttcaatgcacaaggctttcctatgaattgtaaagtgttcagcagcttccctggcctctacctattagatagatgacagtggcatgcctaaattgtggcaacaaagaatgtctccagacatgccaaaatgtaccctgtggaacaaaagtaatcctagttgagaaccactgatctaagttaaaagttgaggctacagcatcaggcttcctgggtttcaatcctcactccacctcttgccagctgttactttatgcttacaaataagttacctaacttgcctatacatcagtttccacatctgtacaatgcagattttaatagcacctacttcaaatgtatgttgtgaagattaaacaagttgatatgtaaagcccttaaatcagtccatgatatatagtgattaacacattcgcattcttactataattagtatttgatttatataaccattcactacttgcaaaactatttcataaacatattctcttatttgatccttatagccactttgtgtgttggcatatgagggaaggttgaggttgttatgtgctctgaaggtctatgttgggtaatgtgtgcttttttaaaaaatatctattttctagttaaggaaactgtagcatagagaaaataaataggattgctagaaatcacacacacgtaacatacaatttaccattttaactattttgtaatgtacagatcagtagcattaagcacattcacattgttgtataaccatcaccaccattcacctgcggagctttcatcatcctaactgaaactccatactcattaaacaataattccccattccttttctcctcatcccctagtaaccaccttactgctttctgtctctctctgtgtatttgactactctaggtacctcatataaatggagtcatacaatatttatacttttgcatctggcttatttcacttagcataatgtcattaaggttcatctatctagtagtatgtgtcagaatttccttcctttctaaggctgagtaatattccattgcatgtatatatcatattttgtttatctgttgatgaacactggggttgttcccacttcttggctattggaagttgctataggctgcatgtgttccttcaaaattcatattatgaaatcctatctcccagtgtgatggttttaggaagtgagacctttgggaggtgattaggtcatgagagctctgccatgggaataattgcccttatacaagagaacacggagggtttccttcctccttctgccacttgaggtgacagtgaaatgacagccatctatcaaccaggaagccagctcttaccagacaaagaatatgccagcgccttgatcttggacttcttgtgagaaataagtttctggaatttataaaccacctggtcaatggtaatttgttacagcagcagacaaagacagaagccaccccactttaaagttgagattcccgactcctggtctaatgctctttccagtattatcacaaagggaactgatggttctgtatcctaaaatctcccccaaactcaagagattttcagggaaatggtcatcatgtgtaaaataattagccagttgaaatattgatgctgagcttttgtgaataaatgaatcaaatagctaagctgggttcattcagactttatctaagtttctttaagcttatctctacattctcctttttcatattgaaaaaaggtaacaatttccagaaagataccaacagatcattagctgatgcagtatctccagaattctttttcttttttttttagaccaggccttgttctgttgcttaggctggagcgcagtggcacaatcacagctcactgcaccacgaactcatggactgaagcaatcctcctgcctcagcctcctgggtagctgggactacagacacatgccaccatatccagctaatttttttctatagtttttttttttttttttgagacagggtcttactatgttgcccagactggtctcgaactcctgggctcaagcaatcctctgcctcagcctcccaaagtgctgggattacagatgtgagccactgcacctggcccctagaattgtttctagaggtgaaacttcaaggtgaaatatagtacataactgcttttcagataaacaagtccagagagcacactctcttgtgctcttggcatcacttggcatcacttcatatttgaggtgtttcaaacccattagaacacgtgaacaaggcctgcttccaaagctggcttccatctggtagtcccattaacaactgggcacaccccttccctagagctctgtgtagacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtaatgtatacatcttccaactttttaaagtcacagagtaagatatgtattttaagaattatttgacttaccatctacttatctttgtatttttgtttttcaaagtttgataaattccctggtcccttagtctgtatatgtgtcaggttagttagatgaagggaatgtaattaagaactaagcagcgatttttatgacatggtgtgcaggttgatagaaagactcaggagccagtctccttccaagctgctaaatgaggcaagtcacatattatctctcagcctgttttcttggctctgaagtggggataataacttaggggatgggcaagaacgggatctgaaaattacagctacaaacaaaagtcaaacgaagaacttgcaacagaaacctttagtgcctcccctcatgcacaagcaacacagttctaaaatatttactgtctgaccctttacagaaaatgtttgccagtccgtagtcaaaaggattaaataagtaatattttcagcacttagcatatgataaacgatacgtggcacatgataaacaataactgtgttaaataaaatatgtgcgcagtgagtcaggcttttccttggacattagtatttttcctgtgttcttacttgtaaacactacattaacaaccccaaataaaactgaaggaactgaaatcttgtatcattttctctaaacttgtaaattctggtaaggccatgaaaatatatgcagagaagtgtttacaggattttaggattggaaaaattgtgaagtactccttgagaatcacattttctgcaaattacagtggttttaattaccattatattattactttctcatgttctttgctgtcatgtttagttgaaacctaaaatgtctcttacacttagagaactaattcttttctgttttttttctgaatagtgaagaatactatacaaaaaagctactacatttttatttaacagatatgagcatttatataatagaggagttgatgtatataaaaatgatttgccatctttttggtctttgaagaaattcgaatgaactttctggaagatagcaagaatttacaaatagagaaaattgttgcctgctgttctcaggcatttgtccaaaaatataaataagtataaatctatgaaaagggcttgatgaaatctaaccttcaaatctctttccagatgtgtatttttggggaaagggctatatttattaagttttttttaaattttaaaatttccagagacaagagaaaagtaaattagaaggaagtcgtattaaaaatgacttaagggcgggtgcagtggctcacacctgtaatcccagcactttgggagacggaggtgggcagattgctggagcccaggagttcaagaccagcctgggcagcacagcaaaacccccaactctacaaaaaatacaaaaattagctgggtgcgggggtgcacacccgtagtcccagctactcgggaggctgaggtgggaggatcgtttcagttcaggaagccaaggctgcaatgagctatgatggcatcattgcactccaagctgggcaatagagccaggctctgtctcaaaaaaaataaaaaaagacttaagaaaaataggtaacccaacctcaaaaattctctttgaatcattaaatttcatggttaaacatttaagctactgaatgattcactctaaggctgtaatgtaactcagatctcctttaggcgaggaagatgctggctgagttttcatcataactggctccttttgccctgtgagatgagagacacagtagcagtttggctcttatgcaatctaaactgttgcgttgggaatacggttcaaaaaacacattggagtttaagctaaagcaagtgttttgctaacaaaaagacaaggcatcacattttgcaattgtctagctcagttataaaacagaagaataggccggacgcggtggctcacgcctgtaatcccagcactttgggaggccgagacgggcggatcacgaggtcaggagatcgagaccatcctggataacacagtgaaaccccgtctctactaaaaatacaaaaaaattagccaggcgtagtggcgggcgcctgtagtcccagctactcgggaggctgaggcaggagaatggtgtgaacccgggaggcggagcttgcagtgagccgagatgacgccactgcactccagcctgggcgacagagcgagactccgtctcaaaaaaaaaaaaaaaaaaaaaactgaagaataattaattcttcaatcaaaacatctgatgaatgctctggtaacttatgctctctactgacctagaaacaaatgagagagtatggtgtggtttgtgcaatctggcagtgagcaagctaccaactaaatcagtgaaagactctcctattctttttttactcttctgcaatcccacaaaaggctatttgaggggatactgactttgagactgggtcctaacatccatgtttggggagttcaggctgctgctccagggtttagcctacagtagcgaaatacaaaggacccagagaccactcattcaaggtttgccctaaatagcagcaacaccactgtcatctcaatacacgaagaatagggcttttcaggtatccttgcctctttgtcacagagaagagtttacagattgtgagacggaaaagtataatttttaaaaccttataatattttctataaaagtcacctgaggtgaaaacttgaaaagaattataattttccagaatgtgagtcaagaaacattagagcaattttatcttaggaaagaggtctttgaatttaggctgaaagtaaattgctctgtctccatgtcctatggttatgggcaagtttggtacataaatgagaaatccatccagtggccttgcccatctcactcccaaacacctgaagaatgtaatgttatatctcctagagtagcagcatggtctccctatgaaagtccttcttctttaaggagacttctttcccttccctcctaggaggatgagtcagaatcatcaagaaaaatatgatgggcagaggcatacagtttaccattaccactagtttagaattactacttagcactttactgcctattacatagttggtgctcaacaaatgtatgataaattaatggttgagtttttctttcttctccatattcatcttccatgacaccacgaagagcaatgtttttcaagaatgttcttcaaggtttgaaagtagcctgctttagagaaactgcctactgtacagcctccaaccaagaggaaaagctgaaaaaagcatgaagggattttgttttgttttgtttgttttggttttaatatgagcattccctggcagaaaagccaggggtaatctcattgcaactaggcaatcactctcaagaaattttctaacaaataaggaggccaatttttattttattttgagacgaagtcccactctgtcacccaggttggagtgcaatggaatgatttcagctcactgcaacctccgcctcccgggttcaagtgattctcctgtctaaacttcccgagtagctgggattacaggctcccaccaccacgcccagctaattttttgtatttttagtagagatggggtttcaccattttggccagactggtctcaaactcctgacctcaagtgatccaccctcctcggcctcctaaagtgctgggattacaggcgtgagccaccacacctgacccaggaggccaatttttaaaaggttaactaatcttcatgtccaaaatgaatgttaattgttcattttggacatgaatgttaattttttttttttttttttttgagacagagtctcactctgttgcccaggctggagtccagtggcactatctccactcactgcaacttcctcctcccaggttcaagcaattatcctgcctcagcctcccaagtagctgggattacaggcccacaccatcaggcctggctaatttttgtatttttagtagagacggggtttcaccatgttggccaggctggtcttgaactcctgacctcgtgatccgccctcctcggccaaccaaagtgctgggattacaggcgtgagccaccgcgcctagccgaatgttaattgtctaaaaatttttcttctccaatgtcttctcctccacttttttcggaatttgtttcttcctaattacagcgcggtgtggaggaaactgcagctgagtgccgaaaactaggcgtcactgcgcatgcgtatgtggtagactgcagcaacagagaagagatctatcgctctctaaatcaggtgagactgcaggttcacaaatttcttcagattattttgtttcctaggacgctgacgtggaaaatgagaaaggtctttatgactgcctgatttaaattggattttagctgctaactgaagtagttatgtcaccaaggaaggatatatactttttttcttgtatgtaatccactcagctctgcccattattattgttcatattattaatcaatttcattctgatcagaagtgtgagcagtggcacagagtgactgacaaaagatttatcatcagggaatatggatcacttcctagttttgttttagtcctattaactttgcagtaattccagcttctctttaattatttcccttgtgagattttattttggtgttaatgtagtcttctgtagaaaatgtaatattaataattattatcacaattattttaaaagagtaaataccaaataatcacaatgaactaagcactctaacaaactttacattttttaattcaatccctacaataactctgtaaacttcattttacagataagcaaattatgactcagagaggttaagccagacccaggtcatgtagttattaggttatgaaaccaggatttctcaaccagcactttagaccaggtgcggtggttcacacatgtaatcccagcactttgtgaggccaaggtggaaggatcacatgagaccaagagttcaagaccagcccaggcaacatagtgagaccctatctctaaaaaaaaaaaaaaaaaaaaaaaaaaaagtttaaagaaaaacacatttttaaaaaatgaacactttaaaaatatttggtcagaatttatataggaatttatcaacataaatgttaatttcactttactgataaacttgcaaaacatgatgtgctgggtactgaaatttagatgttaaaagaacagtttatcccacctttatgacagtgttcccttggcctccacgatttgagctcaacagtctgtcttgcctgaactctgagagacctcatacaatagaagaaagactctcatctttggattatattggtcccaaaactttgagtttgaataatacacccagtgaaagtgttctttcaatttcaaaaggtgaagaaagaagtgggtgatgtaacaatcgtggtgaataatgctgggacagtatatccagccgatcttctcagcaccaaggatgaagagattaccaagacatttgaggtcaacatcctaggacatttttgggtgagtgtgagtcagaaacatttctgatttgtgcaccttctcttaagatacatgaaacttataacggagttcacatacttctggacaggaaactggccagatctttgccttaatcaagaatcattaaatttgtttgagtagaagagccacagagtctctgacacaaggacacagaattcaagtggacacaacacaccagaatgtaagctacttggtctgtcttgtccaccagtatctgacacaaagcttggcatgtaccaggagctcaacaaatgtttgtggaggtttgttaagggttgtcagtgtacatcttttcaatgctgtcacttgtgacttcatttttttccctccacaccatgattttgtaatgtgtcctcattttgtggaattttagaatggaaaggacatcagaagtaattacttggatgtatataggatcgaggacacttttggacgagactctgaggcaagtgttctagatccatggggtgctggaactgagaaatgcagctatacagacctcatataattggttagttttgtgggagatggaaatatcaacttcaactgcctttgtatagaaatttttatgattaatcttccagtgcctcaatattagtgtagaatctagggcagatctggattctagaagaaagaagaaaaaaaagagatgtgtcccccttacctttaccagctcttcacatatgtgaattggctcccatgcccaccaaactacacggagacctcatacattagctacctatagctgcataacaaattatacaaaacttagtggtttaaagcaacaatgtatgttcactatcctctcacagtttctatgggttgggaatttggaggtagcttgggttgggagttctagttctatgaatttgcataggatttattaaattcttataaaattttattgatgtttctcacaaaagaggtttttggaaaaaaagaaagacttgttttctgtaacatcaacatataatatacaatattacaaatagggagatagtgaattcaatcatgattcattagtgtggtgtagaactctcagcttacactactcaactgtcttaatacagttacacaagatttcactcttttaattagaatgataaagccccaaaccaaaaaattatatgacaccaaattatcataaggaataattttagttctgaaaactctgaatttttcccttaatattgtttagatgacatatccaaaaaaggatctatttgattccttctgaagggaaggagggggagtactgagattagtgttggcatggggcttaccataccaataaatttgtatctttatttctatcatttgtaaagaattaatcatggaatgcttggaagtattttatttcattgtataagttctctcaaatgcctttctgtcttaacaaaaataaaactacctgatttggaaacctaacgtctatgtcattgtctttcttctttctgcaatgatccttaagatcacaaaagcacttcttccatcgatgatggagagaaatcatggccacatcgtcacagtggcttcagtgtgcggccacgaagggattccttacctcatcccatattggtaagtatcacatgccagccatgttatatatttttatactttgaagggagcattacacttcaaattgttaccactggagagtcctggttcttggcatcttgaacaaagaattggacaaaactcaccaacaaagccaggaaagaatgaagcaacaaaagcagagatttattgaaaatgaaagtacgctttacagggtgggagtgggcccaagcacaggggctcaagagccaattacagaattttctggggtttaaataccccctagaggtttccactggttacttggtgtacgccctatgtaaatgaagaggatgaattaaagttacagagtcgtttactcagtgtacaccatatgtaaatggagaggatatttcctgtcatagctggagtgtttccatttgatttagttctaggaagtcagcatgaatcggccttatgttccctgcctccagaccctgttctcctgcctcaagattacaatgctgagagcagagtgatttggatttacagaatttaaatttatagtagtttagaatgattttttaaatgactttttctaaaacaatgaaaccaggttgtaattatatttaagatatttttagatttctgcaaactcctctgtagaacaatgagagaaaacagtaatgccaagcatgtttccattgtttcctggaataagaaacagaaaccccacagactgagaagcaaaacctacagaagctaaaatgaacacatgtctatgtcatggccttggtgcccaagataagacaatcagagtggtccctggatcaaaacattttacagtgtgcttgtgccatgaaagtgtgtgtgtgtgtgtgtgtgtgtgtgtgagagagagagagagagagaaaacgactctacctgactaaaagttgcagataccacactccatgcaccaccaaagacataaagggaaggaggtgagaggcgttaaggatgtactgctgtatttgccaaatatcctttcctgtaaactcttctccagatcctcataataaaattaagaggccaaagtggcaaccattgtcaagagaaaaactatcaaccattgtcaagagaataactcagttattgagagagagaggagaaatgagcagagtcctacagaagtctgtcaacacagataccagttttgtagaatttctaaatgtatttttcctgattcatatttttcaaaataaaagcagcaataaaaactgattagaaaacagtttgaagattcaatggaaaaaccttacatgtaggatggaaaactgaacattaagccaatcaatagagttatttttgttcttttgttatcattgttgtttaagaaatgagatacgttcacaattctgcttaatcatgtaagaaaatgaaaatgaattgccatttatactctcagaaaaatcacaagtggctgatttttggcttccacttgttcttaagccaaatgataccgccttctcacagaaagctgaggattggtttcactctcccttagctaacaatgcttaataattctcttacagttccagcaaatttgccgctgttggctttcacagaggtctgacatcagaacttcaggccttgggaaaaactggtatcaaaacctcatgtctctgcccagtttttgtgaatactgggttcaccaaaaatccaagcacaaggtaaggtcaaaatcaagttagaatgggtatgtggtatgataaattgatatgaaaactaatgagaaatgtttaggcaggccaactaatagaagaaaatgaagaaggaaaaataatttttcttattattattattatcttgaaattaaaggaataaagggggaaaacacattagggactagcaggaatgatcagccaccgatgaactgggatatttatttgtgtccgggagaaagcacatacatttgatcaccgttaccaccctgtctttaaaatgcaaatgttccaaggaccagcaaataaattgagtatctagctccttagtcaaggtgaatttctgcaagaactcttgtctctggtgagacaggatttgagaccacaagagaagaaaaattagtcctgaaaggagaagaaaaaagcaggaaggtgtggataagaacccgaaaattaagccatctgcttaacaaatttttctaatcctagtatatattctgctgcaggttaacaaaatatactaagcttaatgattcgaaaccaattttttactggaagggaattaatcctaaatatattcattcaaaagaactaaacaattctctgttgagtgccgcctcatttgaggatactgactcttacagcctgagttagctatgtggtctctgcagctggaatcactccctgccactggagtccttcatggtgttagaccataggtactgttgactaaagaaaaaaaaaagtttttgtttttatttttgttttttttgagacagagtctcactctgtcacccaggctggagtacagtggcgcgatctcagctcaccgcaacctccgcctttctgggttcaagcaattctccttcctcagcctcctgagtatttggattacaggcgcccaccaccacgcctggctaatttttgtatttttagtagagacggggtttcaccatgttggccaggctggtctcaaactcctgacctcaggtgtcctacctgccttggcctcctaaaatgctgggattacaggagtgagccaccatgcccggccaaaaaaataagtttttaaagaattaaaggtcatcctggctaacacagtgaaaccccgtctctactaaaaaacacaaaaaaattagccgggcgtggtggcgggcgcctgtagtcccagctgcgcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaaaaaaaagaattaaaggtgttaattttatttagaagccttactgaagactacagtcggaggcctatagcctgagagcagccctttagagaggttcagttgaactgttctgatagtgggggccatgtgctctatcctgtattgtcttcaaagcatctttccagagagctgcacattgtcacagagtcagggactttgtgaaattatgctgacaaccagaagtgagtaaacgtggcttcttacatttgctacgttgtctcacagtacttaataagtatgcaatatgtaagtaaatactatagtactattgcaactcctgattgttttcttagacaaggaattgggcccaataaaaaccctcttggtaggcattcaggcttcgtgtaccatgagctttcctaagggtatcctgccactcttggggaaggcatgatagatgaggggagtaaggataatggaactctgggtacagggttcctgggggctaacttagaggtagacacaggcaatgctaaatatttgggattgattttatagaggttgctagattgtgaatttccttagtaagggctaaggcattgatatgtaatgtcacacttggctccgaggctgggttgttggatccatgtagatgaaatcagggagagaaagggcagaacggagtaatttagaaatgtattgatttgtattactctctgttggcttgctattcaaggcagtggagaactcaatcacataataatctgcagcaaaccacagatcatcccagggaatgaagttttaacattcgctggctccctaactcctcacccagcctttacattcactggctgttcagtccatgcctggacatcttaatttgaatacaacattttaaatccatttttctgtcatcatcttgcactaacagacaattctacactaagcctatgtttatgaatatttctcaagagtacatgtacacagccttcagtataaggaaaactggaagtatgacatacctccagttgtcatactccttgggcccctcttaaattctcattaaactgcaggataggcaagtcagaggtgaatctcaaatacgaaattcttaccggaaaggggttccaatccagaccccaagagagggttcttagatttctcgcaagaaataattcggggcaaggccacagtgcaaagcaaaagcaagtttattaggaaagtaaaggagtagagaacagctactccatggagaagaatggcttgagctgctccaccaagggtatttagagttacttcttgattatatgctaaacaaggggtggattattcatgagttttccgggaaaagggtgagcaattcccagaactgagatttcctcccctttttaggccatatagggtaacttcctgccattgccatggtatttgtaaactgtcatagtgctggtggaagtgtctcttagcttgctaatgtattatagttagcttataatgagcagtgaggacaaccagaggtcactttcatcaccatcttggttttggtgggttttggccggcttctttactgcaccctattttatcaacaaggtctttatgacctgaatcttgtgccaacctcctatctcatcctgtgacaaagaatgccttaacttcctgggaatgcagcccagtaggtgtcagccttattttacccagaccctattcaagatggagttgctctgatttaaacgcctctgacaaaatgacgacctcaaaacaatccagctttatggaatacctccacaagaaagaaagtatacttagctatagaattttctccttgcatccaacaggctttgagatgtcagatgtttccttcctgtcccatgattaatcctagccattcctctttcttgtctggctccactactccttaccatctaatgcctcgccaccattttgatattttgactaagtgagctatgaaacacacctactggatatgaaagtataagtttctgataacaaaacatcaacatgggatgtggaggaagtgggtagggtggcattaatgcagcaaatcctggaatattttaaatcttcattctaaatttagtaaaaatataggataattttcctgccatcatttacttataaaattaaaattttagaaaataaaaataatattttcctctttttaatcacagattatggcctgtattggagacagatgaagtcgtaagaagtctgatagatggaatacttaccaataagaaaatgatttttgttccatcgtatatcaatatctttctgagactacagaagtaagtacagcacagaacacccaaatactaaaacaccaatagagcttttttttttgctttttttttttttagacagagtctcactctgtcaccctggctggattgcggtggttgcagtggcatgatcttggctcactgcaacctccgcctcctgggttcaagcaattctcatgcctcagacccccaagtaactgggattataggtgtgtgctgccacactacacccagctaatttttgtattttttgatagagacaggtttccccatgttggccaggctggactcgaactcctgacctcaagttatcctcctgtctcggcctcccaaagtgctgggattacagtcatgagccaccatgcctggcccaatagagctattattatggagcatctttcagttgtgaaaattggcatggaaactctccatccctggggagaacagttatttcctctgttattttcctacccagtctataaaaagagagtgattcattttctctaccaaatctactgtctctgcccaaactttgctgaagactattctaactaaaggaaacacagtttaaaaagaatgcaatatagtgaagtagttaataataaagactccatttttaaaagtctgctggaagtttggttgggattgcactgaatctatagagcaattggggagtattgacatatcaacaatattgagttttctaatccaagaacataatatctatttttaaaatcttcttcaaaatctttaaatctttaaattgtattttgtagtttttggtgtttaagtcttgcacatattttgtcagatttattccaaagtatttcacgggttcttttttttttttttttttttttttttgagacagagtttcacccttgttgcccaggctggagtgcagtggcgtgatcttggctcactgcagcttctgcctcctggcttcaagtgattctcctgcctcagcctcccaagtagctgggattacaggcacctgccccctcgcccaactaactttttgtgtttgtagtagagacagggtttcaccatgttggccaggctggtctcgaactcctgacctcatgtgatccacctgcctcagcctcccaaagtgctgggattacaggcatgagccatcatgcccagccctatttgacggtttttgacgctaatgcaagtggcattttaaaaaattttatatttcccattgtttgttgtcagtatatattggatttttgtaatttgatctcatattttgcagtcttgctaaattgctaaacctctttttgctaaactcgataagcttttttttttttggtagattcctgggcctctaattttctttatgggaaagtttttaattacaaatttaatttctttaatagctacatggctattcaatttacttattaattcttggtaatgtgtgtctttcaaggaatttgtccatttcatctaagttgtagaatttctttggcataaatttgtacataacattcccttattatccttttaatgtctttagaatgtcttatttatttatttatttatttttattatatttttttgagacagagtctcgctctgttgcccaggctggagtgcagtggcacaatcttggctcactgcaagctccgccttctgggttcatgccattctcctgcctcagcctccctagttgctgggactacaggcgcctgcaaccatgcccagcttattttttttttttttttttttttttttttttttttttttttttttttagtagagacggggtttcaccctgttagccaggatggtctcgatctcctgacctggtgatccgcccgcctcagcctcccaaagtgctgggattacaggcgtgagccaccaagcccagcctatttatttatttagtagagacagtctcactttgctgcccaggcaacaaaggttttgaatgcctggcctcaagcagtcctcctgccttggcctcccaaagtgctgggattacaggcatgagccactgcacctggccaaatgaatatgctgataatatcttctttataaggatgacataagaataaaataatgtaatacaaacaaagcccctgtcactgaaaatgtatagacttcaaatgttaaagtcttagagaacagaatttatatgaaatagcaacagcaacaatttcccagaggaaatactctctcagctttcttctgaggagcagtttctaaattgaaattgtatcagtgagaagataactatactaacttcataagccttgggcctttttgaaacaaatccatataaactatgaacaaacttgaaatagaacaatttgagaacagggtacaaactgcattggtgtatcaatttcagtatttggttttagcttaaatagactgacttgagataacataaggagaaccttgacccccaagcaacatcatctcgcgagttgactaggccgggtgtggtgtctcacgcctgtaattccagcactttgggaggccacagcaggcagatcacttgaggtcaggcattcgagaccagcctggccaacatggtgaaacctcagctctactaaagatacgaaaattagcaggcatagtggcctgcacctgtaataccaggcactcgcaggagaatcccttgaacccggaaggcggagattgcagtaaaccatgattgtgccactgcactccagcctgggcaacaggagactctgtctcggaaaaataaattttttaaaaaaatgaaaaaaaataaaagttgactaaattagtgtcttggtactaagcactgtaggaagtgagtttcatggaaccccaactctcttggggcccaaagcaagtcatattaatattgaaaattacatgcatatacatgcatatgaccaaggtgataaaaacaattattctgcctgagttggagaatagtatcccagtaaaataaacaagagtctcaaagtcttttgtatcctttgaagctgtcatggtggtttgtaactaggcaacaggtatatattgttaatcttctttgcatttaattccttttatagagagacacaattttacgagcagatgcaattactagcatgaaggtttctttgtgagggtagttaaaaggcccacatgagctctcttcttatccttgtccttctttcagccagatcttccctgcccctttgctcattccatctttcacccacctacccccaaaacaaggaagtaaatcttgcattagtcaacaataccaaagtgattttcaatatgactttctctgcagaatgttattatttctgcctctttacattcacatactgtcttcctttttttttttttttttttttttttttttagattgggtctcactctgttgcccaggctggagtgcagtggcttgatctcagctcactgtaacctccacctcctgagttcaagcaattctcctgcctcagcctcctgagtagctgggattacaggcatgtgccaccacacctggctagtttttttgtatttttagtagagacagggtttcaccatgttggtcaagctggtctcgaactcctgacctcatgatctgaccacctgtgcctctcaaagtgctgggattacaggcgtgagccaccgggccagccactctcttcctttcagttgcctactcatctcttatgcattcctggacatcagttgtccttttgaagctttcctccactatcccagcccatgtgaatcctccttccagttatagcccttaattctagatggctgatatttttcaataattgttttaagatgaccattttagcctatcagctaaacaatatcaaagacaatagctatttttcaagtactttagtttaccttattatagagtgcataatagatattcagtaaatagtaaaggagaggtgaaggcttgcatagaatggattctggtggtgtctcttggtgagcttttagcatcaagattaatcagcagtttcagcaatgagctcagaccttcagttttagatctttactcatatcagataagagagtgagaagagtggtatgtatcagtgctttatttatatttgcatccaatttgaactatgaatattacaaaggtgcacacataggttcagacagattgatttaaaatgaccaaagatgacctgtcgtaagcaacctgggtatcttaagatgcactccttggagagggaatgttcctaaaaacattttcagagggacgaactgtatgaaattcagtaaaacataaatcatgaggaaaactgattactctctttttgacatgaaatgagagttttaatgcatggttacgattattaacgtactccgctgcaagacgttaataaagttactgttttgcaggctagaatgtcttgatgctgtaatcagaacacactttttcccctttcttccagcttcaaatgcagattcataattgggctgacttctaataactgcaatgttttctgccttgggcttgcagcagaagcctgacaaaatagtgtttgtttaggcaataatttatttatttatttattgagatggagtttcattcttgtcgcccaggctggagtgcaatggcgtgatctcggctcactgcaacctctgtgttcaggcaataatttagactttaccttacttgtgattactatagcaattactatagccacaaggcataattttactgtctcatttcaattttatgaatttgaatgtttttacacttttcctaatgaagtccactatgaagttatgtcaaaaaaaaaaaagaaaaagaaagatgcacacgtaaaagagaggtggttgcaagagaagaaaagaacggaggaaagttaaacgcaaaccagataactctcagcgtattctaaatgaccaaaaacagaactctgttgtcaaagattttaaatggaaaatttttcaatttttttttcttttttgtacaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctgtctctatgagtgtccttcaagaaccaaggatgtggatggtaacagctacatctggcagttattaaatgaatccaggggttttctcaactctggtttctttgacagcaggagtgctggtttatcaactggaacttactgacccctttctatgtgtgagattaagcacagtgctattgtttctaattttttttcttagtcaccggcaataatttttttcataacattcttttaatttcttgccttataatcaagaaatttatgagtaaaatgtattatttttaaagtatggttacaaaaattcaaaagagaaacatggaaagtaaaaaatagaagcttacccccagcaatcccatttcccagaggtcacactgtcaatgcacaatcttccagtcttttactgatgcatgttcatgacaaatacattatattcttattgcatgggtcgtatcagtagtttcataccatacattatgttctgtaacttgctcttttagcttaccaacttatcacaaacagctgtttgtttcagtatagagaattcaactttttttcatgagtacacagtactaaaatggtactctaaccattcccttttgataggcacttaggatgtttccagtatttcttagttcatctatttaaatattaatttaacaaatactgagtgccctcaaagttctaggcccagggaaacatatatgaataaggcaaactccttgtcttaacagagctcacattctagtaggtgggacaaacaataaagggactgcaaatatgagagagagcatttaaagtagtagagtcagggaagagcacgctgagaaagaacattttgcggacgaactattaccatacacaggcaactaaaaaatattttatgatatacatattgacaaaaatagaaacctcttaaattccagtgttaagttcagagcagaaaacttcttattttaattccattttggttttaatcttttatgaatgaagctcagagaaatgtgcgagcaaaatattcatctctctttgtccatttttcttgcttattttgagtgtagtcctcctccttaaaagttccacctgcagccaggcgtggtgactcatgcttgtaatcctagcactttgggaggccaaggggggcagatcacctgaggtcaggggttcgagaccagcctggccaacaaggcgaaaccctgtctctactaaatatacaaaatttagctgggcatggtggtacatgcctgtaatcccagctactcaggaggctgaggcaggagaatcacttgaacctgggaggcggaggttgcagtgagctaagatcacgccaccgcactccagcctgggcaacagagtgagactccgtctcaaaaaaataaaaagctccacctgagcttccaaatgttttcctatgacaatcttcatctataattctctctaagagccctctctttcaccaaaatcccagttccaagggctactgctaggatgtgcagatcaccagacaaatatttttttgcagggcttctatctatataaacacttttattaaaaatgctttataaaatttataggtccatgtgcatgcagtatagtgattttttgataaagatttagaataataggaagatttgtgtatttattgtccgagcagtacatgtaaagattcttctcggcatcctggtactatctctttggtccaggaatcatttcttttttatcaaatgagccattcctagctaatttcatatcttttaccacaagaaagggtagcaatgctgttattattcataatgccatggctctttgtaatatgtttgtaataaaataatatgaatcttcacccctttccaataccctgctaccatttatttaatcttagctacattatgccttttgacattgcatcatccattgtgctacccatgaatactgggtatcactcagagacaatcactcaaagattgttactggtacaacacagttcaatatagtacaattgttactggtacaatgtttgatgataatcacaaattcaagtcttacccagggtatatatacgagaaaacatgaatgattcgttgtttattgctcattttaaatttatcatttagaatgttatagcatcacatagaacaacacatctttcaactgtgtcttgtcttgaactttttaggccatcatcattacatttctagaattgatctctgccactcctgaccgtaccctacctcccacacaccaccagcagcagagaggggattgtggacagaacaaaatgaacagccccatttgcacaaggaatgtctgtccctcctctaggagagcttaacattgaccagggagaagagaacacagcatcatcacatcagtcagaaaggcccactgttgtgaagaagcaatagtcttaaaagccctcagaagagataagtgcagccaccccccaacccccagccaaaaggactgcctgacccatggcatagaacctgcagaggggataggatgattttcctgaatgccattggcaagcggggtaaggacatctgatgtcaccctgcagcccttggatgttatatagcagaagtggcatcatactcagaacaaactgcgagtggatgcatgggtcaacacatgcggcttgggcagtggaccttagatagcttacaaccctaaattagatctatgcccaatgtgagtgagaattatccaaaatcagcatgtcagcatcattctggcagccaaatttcatgtaatcctgcaaaagaaattctttgccaaccagaagtagcagtct

SEQ ID NO: 7 - DNA, 21 bp, Artificial sequence
  Primer
aataagcgtggtgttgaggaa

SEQ ID NO: 8 - DNA, 22 bp, Artificial sequence
  Primer
cgacatcacctacttctctctt

SEQ ID NO: 9 - DNA, 23 bp, Artificial sequence
  Probe
ttgtaaatctcggcccggttgct

SEQ ID NO: 10 - DNA, 16 bp, Artificial sequence
  Synthetic oligonucleotide
aggtattatgggctgc

SEQ ID NO: 11 - DNA, 16 bp, Artificial sequence
  Synthetic oligonucleotide
agaacggtctgcccgg

SEQ ID NO: 12 - DNA, 16 bp, Artificial sequence
  Synthetic oligonucleotide
ttgtaaatctcggccc

SEQ ID NO: 13 - DNA, 16 bp, Artificial sequence
  Synthetic oligonucleotide
cacgatctcgacatca

SEQ ID NO: 14 - DNA, 16 bp, Artificial sequence
  Synthetic oligonucleotide
cactaagaaggtctgc

SEQ ID NO: 15 - DNA, 16 bp, Artificial sequence
  Synthetic oligonucleotide
ccccaaggtgtccagt

SEQ ID NO: 16 - DNA, 16 bp, Artificial sequence
  Synthetic oligonucleotide
cagtaatagtagcaca

SEQ ID NO: 17 - DNA, 16 bp, Artificial sequence
  Synthetic oligonucleotide
tttatacagtcagagt

SEQ ID NO: 18 - DNA, 16 bp, Artificial sequence
  Synthetic oligonucleotide
atattttggcagaagg

SEQ ID NO: 19 - DNA, 16 bp, Artificial sequence
  Synthetic oligonucleotide
tcccattacatgggtt

SEQ ID NO: 20 - DNA, 16 bp, Artificial sequence
  Synthetic oligonucleotide
taccaaggcatgggta

SEQ ID NO: 21 - DNA, 16 bp, Artificial sequence
  Synthetic oligonucleotide
gttagaaacacctatt

SEQ ID NO: 22 - DNA, 16 bp, Artificial sequence
  Synthetic oligonucleotide
gagaatcaatccctca

SEQ ID NO: 23 - DNA, 16 bp, Artificial sequence
  Synthetic oligonucleotide
tattattctttaccct

SEQ ID NO: 24 - DNA, 16 bp, Artificial sequence
  Synthetic oligonucleotide
taattgttgtaccgct

SEQ ID NO: 25 - DNA, 16 bp, Artificial sequence
  Synthetic oligonucleotide
tccggtacatgacagc

SEQ ID NO: 26 - DNA, 16 bp, Artificial sequence
  Synthetic oligonucleotide
tattttttacgaggga

SEQ ID NO: 27 - DNA, 16 bp, Artificial sequence
  Synthetic oligonucleotide
aagtattgatgtcttc

SEQ ID NO: 28 - DNA, 16 bp, Artificial sequence
  Synthetic oligonucleotide
ataattaatctggagc

SEQ ID NO: 29 - DNA, 16 bp, Artificial sequence
  Synthetic oligonucleotide
ccactaatgttggctt

SEQ ID NO: 30 - DNA, 16 bp, Artificial sequence
  Synthetic oligonucleotide
cttacctaagattgtc

SEQ ID NO: 31 - DNA, 16 bp, Artificial sequence
  Synthetic oligonucleotide
agttatcgaagatgct

SEQ ID NO: 32 - DNA, 16 bp, Artificial sequence
  Synthetic oligonucleotide
agcataaactaggcca

SEQ ID NO: 33 - DNA, 16 bp, Artificial sequence
  Synthetic oligonucleotide
agaactaataggcatg

SEQ ID NO: 34 - DNA, 16 bp, Artificial sequence
  Synthetic oligonucleotide
cggtattaattcatac

SEQ ID NO: 35 - DNA, 16 bp, Artificial sequence
  Synthetic oligonucleotide
gtgctatagtaatttt

SEQ ID NO: 36 - DNA, 16 bp, Artificial sequence
  Synthetic oligonucleotide
taaattcctagagccc

SEQ ID NO: 37 - DNA, 16 bp, Artificial sequence
  Synthetic oligonucleotide
acacggttattaggtg

SEQ ID NO: 38 - DNA, 16 bp, Artificial sequence
  Synthetic oligonucleotide
cctataattaatccct

SEQ ID NO: 39 - DNA, 16 bp, Artificial sequence
  Synthetic oligonucleotide
gcatatatggagctat

SEQ ID NO: 40 - DNA, 16 bp, Artificial sequence
  Synthetic oligonucleotide
cttgttaagtacctat

SEQ ID NO: 41 - DNA, 16 bp, Artificial sequence
  Synthetic oligonucleotide
cgtgtataactgagaa

SEQ ID NO: 42 - DNA, 16 bp, Artificial sequence
  Synthetic oligonucleotide
tcagggttctgcgagg

SEQ ID NO: 43 - DNA, 16 bp, Artificial sequence
  Synthetic oligonucleotide
cgtaacaaatcaccca

SEQ ID NO: 44 - DNA, 16 bp, Artificial sequence
  Synthetic oligonucleotide
agcggttaaacatggc

SEQ ID NO: 45 - DNA, 16 bp, Artificial sequence
  Synthetic oligonucleotide
cttactcaagtccagt

SEQ ID NO: 46 - DNA, 16 bp, Artificial sequence
  Synthetic oligonucleotide
attcatatgtcaagga

SEQ ID NO: 47 - DNA, 16 bp, Artificial sequence
  Synthetic oligonucleotide
gatacttatattcagc

SEQ ID NO: 48 - DNA, 16 bp, Artificial sequence
  Synthetic oligonucleotide
tatattagatgacaga

SEQ ID NO: 49 - DNA, 16 bp, Artificial sequence
  Synthetic oligonucleotide
aaggatagtttaatct

SEQ ID NO: 50 - DNA, 16 bp, Artificial sequence
  Synthetic oligonucleotide
aataggtgaaggagtt

SEQ ID NO: 51 - DNA, 16 bp, Artificial sequence
  Synthetic oligonucleotide
agtgaacacacctagc

SEQ ID NO: 52 - DNA, 16 bp, Artificial sequence
  Synthetic oligonucleotide
gataggttgatcagga

SEQ ID NO: 53 - DNA, 16 bp, Artificial sequence
  Synthetic oligonucleotide
gagcatatattaatgg

SEQ ID NO: 54 - DNA, 16 bp, Artificial sequence
  Synthetic oligonucleotide
cagattaatgctagag

SEQ ID NO: 55 - DNA, 16 bp, Artificial sequence
  Synthetic oligonucleotide
caggtattatgggctg

SEQ ID NO: 56 - DNA, 16 bp, Artificial sequence
  Synthetic oligonucleotide
gaactttaccagtgac

SEQ ID NO: 57 - DNA, 16 bp, Artificial sequence
  Synthetic oligonucleotide
tccgcggtttcctcaa

SEQ ID NO: 58 - DNA, 16 bp, Artificial sequence
  Synthetic oligonucleotide
atctcggcccggttgc

SEQ ID NO: 59 - DNA, 16 bp, Artificial sequence
  Synthetic oligonucleotide
acagagttgtaaatct

SEQ ID NO: 60 - DNA, 16 bp, Artificial sequence
  Synthetic oligonucleotide
ccacgatctcgacatc

SEQ ID NO: 61 - DNA, 16 bp, Artificial sequence
  Synthetic oligonucleotide
ggatatatcgccccgg

SEQ ID NO: 62 - DNA, 16 bp, Artificial sequence
  Synthetic oligonucleotide
gcactaagaaggtctg

SEQ ID NO: 63 - DNA, 16 bp, Artificial sequence
  Synthetic oligonucleotide
gtatgagggcttgcct

SEQ ID NO: 64 - DNA, 16 bp, Artificial sequence
  Synthetic oligonucleotide
tcagtaatagtagcac

SEQ ID NO: 65 - DNA, 16 bp, Artificial sequence
  Synthetic oligonucleotide
gacattatctacaata

SEQ ID NO: 66 - DNA, 16 bp, Artificial sequence
  Synthetic oligonucleotide
ggtttatacagtcaga

SEQ ID NO: 67 - DNA, 16 bp, Artificial sequence
  Synthetic oligonucleotide
cttaagtgttgtaaat

SEQ ID NO: 68 - DNA, 16 bp, Artificial sequence
  Synthetic oligonucleotide
ctgaatcccatctgtc

SEQ ID NO: 69 - DNA, 16 bp, Artificial sequence
  Synthetic oligonucleotide
tataccaaggcatggg

SEQ ID NO: 70 - DNA, 16 bp, Artificial sequence
  Synthetic oligonucleotide
actcatataccaaggc

SEQ ID NO: 71 - DNA, 16 bp, Artificial sequence
  Synthetic oligonucleotide
gtaagttatgtggctt

SEQ ID NO: 72 - DNA, 16 bp, Artificial sequence
  Synthetic oligonucleotide
tattttaggattgctg

SEQ ID NO: 73 - DNA, 16 bp, Artificial sequence
  Synthetic oligonucleotide
gtattattctttaccc

SEQ ID NO: 74 - DNA, 16 bp, Artificial sequence
  Synthetic oligonucleotide
gataattgttgtaccg

SEQ ID NO: 75 - DNA, 16 bp, Artificial sequence
  Synthetic oligonucleotide
atattttttacgaggg

SEQ ID NO: 76 - DNA, 16 bp, Artificial sequence
  Synthetic oligonucleotide
atctttaatgtgacct

SEQ ID NO: 77 - DNA, 16 bp, Artificial sequence
  Synthetic oligonucleotide
cttacggtgaaaccta

SEQ ID NO: 78 - DNA, 16 bp, Artificial sequence
  Synthetic oligonucleotide
gactttaaggagggtt

SEQ ID NO: 79 - DNA, 16 bp, Artificial sequence
  Synthetic oligonucleotide
gctaatttttagccta

SEQ ID NO: 80 - DNA, 16 bp, Artificial sequence
  Synthetic oligonucleotide
tcctataagttatcga

SEQ ID NO: 81 - DNA, 16 bp, Artificial sequence
  Synthetic oligonucleotide
cttgataaatcatctt

SEQ ID NO: 82 - DNA, 16 bp, Artificial sequence
  Synthetic oligonucleotide
cagcataaactaggcc

SEQ ID NO: 83 - DNA, 16 bp, Artificial sequence
  Synthetic oligonucleotide
acaaatattgtgacct

SEQ ID NO: 84 - DNA, 16 bp, Artificial sequence
  Synthetic oligonucleotide
gtaactcaattgtgaa

SEQ ID NO: 85 - DNA, 16 bp, Artificial sequence
  Synthetic oligonucleotide
ccagaactaataggca

SEQ ID NO: 86 - DNA, 16 bp, Artificial sequence
  Synthetic oligonucleotide
ccacggtattaattca

SEQ ID NO: 87 - DNA, 16 bp, Artificial sequence
  Synthetic oligonucleotide
agtatatagggtccct

SEQ ID NO: 88 - DNA, 16 bp, Artificial sequence
  Synthetic oligonucleotide
ctctatcctggcccac

SEQ ID NO: 89 - DNA, 16 bp, Artificial sequence
  Synthetic oligonucleotide
attaggtggattccag

SEQ ID NO: 90 - DNA, 16 bp, Artificial sequence
  Synthetic oligonucleotide
tcctataattaatccc

SEQ ID NO: 91 - DNA, 16 bp, Artificial sequence
  Synthetic oligonucleotide
atgcatatatggagct

SEQ ID NO: 92 - DNA, 16 bp, Artificial sequence
  Synthetic oligonucleotide
acatcgacaaacttgt

SEQ ID NO: 93 - DNA, 16 bp, Artificial sequence
  Synthetic oligonucleotide
ctttttagattatcct

SEQ ID NO: 94 - DNA, 16 bp, Artificial sequence
  Synthetic oligonucleotide
cgtactaagatttgct

SEQ ID NO: 95 - DNA, 16 bp, Artificial sequence
  Synthetic oligonucleotide
cagcggttaaacatgg

SEQ ID NO: 96 - DNA, 16 bp, Artificial sequence
  Synthetic oligonucleotide
gcttttaaggcacgct

SEQ ID NO: 97 - DNA, 16 bp, Artificial sequence
  Synthetic oligonucleotide
tatgtatacggttggg

SEQ ID NO: 98 - DNA, 16 bp, Artificial sequence
  Synthetic oligonucleotide
attatatgctccggaa

SEQ ID NO: 99 - DNA, 16 bp, Artificial sequence
  Synthetic oligonucleotide
gattttagtggcagcc

SEQ ID NO: 100 - DNA, 16 bp, Artificial sequence
  Synthetic oligonucleotide
agtactaacaatgcag

SEQ ID NO: 101 - DNA, 16 bp, Artificial sequence
  Synthetic oligonucleotide
tgatttacccagtggt

SEQ ID NO: 102 - DNA, 16 bp, Artificial sequence
  Synthetic oligonucleotide
aaggcataattcatta

SEQ ID NO: 103 - DNA, 16 bp, Artificial sequence
  Synthetic oligonucleotide
tgactaaatatgcctc

SEQ ID NO: 104 - DNA, 16 bp, Artificial sequence
  Synthetic oligonucleotide
atttttaacctacgca

SEQ ID NO: 105 - DNA, 16 bp, Artificial sequence
  Synthetic oligonucleotide
ccagattaatgctaga

SEQ ID NO: 106 - DNA, 16 bp, Artificial sequence
  Synthetic oligonucleotide
ggactattgatcttca

SEQ ID NO: 107 - DNA, 16 bp, Artificial sequence
  Synthetic oligonucleotide
tattatgggctgctgc

SEQ ID NO: 108 - DNA, 16 bp, Artificial sequence
  Synthetic oligonucleotide
aagaactttaccagtg

SEQ ID NO: 109 - DNA, 16 bp, Artificial sequence
  Synthetic oligonucleotide
ctgcatttgtccgcgg

SEQ ID NO: 110 - DNA, 16 bp, Artificial sequence
  Synthetic oligonucleotide
taaatctcggcccggt

SEQ ID NO: 111 - DNA, 16 bp, Artificial sequence
  Synthetic oligonucleotide
accacgatctcgacat

SEQ ID NO: 112 - DNA, 16 bp, Artificial sequence
  Synthetic oligonucleotide
tggatatatcgccccg

SEQ ID NO: 113 - DNA, 16 bp, Artificial sequence
  Synthetic oligonucleotide
tggcactaagaaggtc

SEQ ID NO: 114 - DNA, 16 bp, Artificial sequence
  Synthetic oligonucleotide
gctcggtggaagccca

SEQ ID NO: 115 - DNA, 16 bp, Artificial sequence
  Synthetic oligonucleotide
cttccggctctaatac

SEQ ID NO: 116 - DNA, 16 bp, Artificial sequence
  Synthetic oligonucleotide
taatagtagcacattt

SEQ ID NO: 117 - DNA, 16 bp, Artificial sequence
  Synthetic oligonucleotide
atcagtaatagtagca

SEQ ID NO: 118 - DNA, 16 bp, Artificial sequence
  Synthetic oligonucleotide
aaaaatcagcccttat

SEQ ID NO: 119 - DNA, 16 bp, Artificial sequence
  Synthetic oligonucleotide
gtacaatgatcagagg

SEQ ID NO: 120 - DNA, 16 bp, Artificial sequence
  Synthetic oligonucleotide
atataccaaggcatgg

SEQ ID NO: 121 - DNA, 16 bp, Artificial sequence
  Synthetic oligonucleotide
gatactcatataccaa

SEQ ID NO: 122 - DNA, 16 bp, Artificial sequence
  Synthetic oligonucleotide
agattttatcccaatg

SEQ ID NO: 123 - DNA, 16 bp, Artificial sequence
  Synthetic oligonucleotide
gtattttaggattgct

SEQ ID NO: 124 - DNA, 16 bp, Artificial sequence
  Synthetic oligonucleotide
agacttaaggtagtta

SEQ ID NO: 125 - DNA, 16 bp, Artificial sequence
  Synthetic oligonucleotide
agataattgttgtacc

SEQ ID NO: 126 - DNA, 16 bp, Artificial sequence
  Synthetic oligonucleotide
gatttgataatctcag

SEQ ID NO: 127 - DNA, 16 bp, Artificial sequence
  Synthetic oligonucleotide
tatattttttacgagg

SEQ ID NO: 128 - DNA, 16 bp, Artificial sequence
  Synthetic oligonucleotide
agttacacttgcagct

SEQ ID NO: 129 - DNA, 16 bp, Artificial sequence
  Synthetic oligonucleotide
agagataatgatgggt

SEQ ID NO: 130 - DNA, 16 bp, Artificial sequence
  Synthetic oligonucleotide
tcatttgggccttgcc

SEQ ID NO: 131 - DNA, 16 bp, Artificial sequence
  Synthetic oligonucleotide
agtcttaactgagtat

SEQ ID NO: 132 - DNA, 16 bp, Artificial sequence
  Synthetic oligonucleotide
ttcgggttaaggcttt

SEQ ID NO: 133 - DNA, 16 bp, Artificial sequence
  Synthetic oligonucleotide
attatacgcaaaccaa

SEQ ID NO: 134 - DNA, 16 bp, Artificial sequence
  Synthetic oligonucleotide
gatatcgatctgactt

SEQ ID NO: 135 - DNA, 16 bp, Artificial sequence
  Synthetic oligonucleotide
agtctaagattgatac

SEQ ID NO: 136 - DNA, 16 bp, Artificial sequence
  Synthetic oligonucleotide
aaatttgtgagctaca

SEQ ID NO: 137 - DNA, 16 bp, Artificial sequence
  Synthetic oligonucleotide
aatagtaaggaattgg

SEQ ID NO: 138 - DNA, 16 bp, Artificial sequence
  Synthetic oligonucleotide
tagtatatagggtccc

SEQ ID NO: 139 - DNA, 16 bp, Artificial sequence
  Synthetic oligonucleotide
gcaatattgtcaaggg

SEQ ID NO: 140 - DNA, 16 bp, Artificial sequence
  Synthetic oligonucleotide
gttattaggtggattc

SEQ ID NO: 141 - DNA, 16 bp, Artificial sequence
  Synthetic oligonucleotide
gatcttaaggtccacg

SEQ ID NO: 142 - DNA, 16 bp, Artificial sequence
  Synthetic oligonucleotide
cagggatatgctgcag

SEQ ID NO: 143 - DNA, 16 bp, Artificial sequence
  Synthetic oligonucleotide
gtagggttgtgtttgc

SEQ ID NO: 144 - DNA, 16 bp, Artificial sequence
  Synthetic oligonucleotide
cttcttaatcaggttt

SEQ ID NO: 145 - DNA, 16 bp, Artificial sequence
  Synthetic oligonucleotide
gtgcgattgtgatgcc

SEQ ID NO: 146 - DNA, 16 bp, Artificial sequence
  Synthetic oligonucleotide
acattcgagatgcaca

SEQ ID NO: 147 - DNA, 16 bp, Artificial sequence
  Synthetic oligonucleotide
gtgcgatttctacaga

SEQ ID NO: 148 - DNA, 16 bp, Artificial sequence
  Synthetic oligonucleotide
gcaaaattggatgacg

SEQ ID NO: 149 - DNA, 16 bp, Artificial sequence
  Synthetic oligonucleotide
cgcttttaaggcacgc

SEQ ID NO: 150 - DNA, 16 bp, Artificial sequence
  Synthetic oligonucleotide
aaaattatatgctccg

SEQ ID NO: 151 - DNA, 16 bp, Artificial sequence
  Synthetic oligonucleotide
gtagattaaaaggtga

SEQ ID NO: 152 - DNA, 16 bp, Artificial sequence
  Synthetic oligonucleotide
gaattctatggtgtct

SEQ ID NO: 153 - DNA, 16 bp, Artificial sequence
  Synthetic oligonucleotide
aagaatacaggacttc

SEQ ID NO: 154 - DNA, 16 bp, Artificial sequence
  Synthetic oligonucleotide
atcaaggaacaaccag

SEQ ID NO: 155 - DNA, 16 bp, Artificial sequence
  Synthetic oligonucleotide
gctagtaatgactttc

SEQ ID NO: 156 - DNA, 16 bp, Artificial sequence
  Synthetic oligonucleotide
aactatacatggctct

SEQ ID NO: 157 - DNA, 16 bp, Artificial sequence
  Synthetic oligonucleotide
gcatatattaatggtt

SEQ ID NO: 158 - DNA, 16 bp, Artificial sequence
  Synthetic oligonucleotide
aatttttaacctacgc

SEQ ID NO: 159 - DNA, 16 bp, Artificial sequence
  Synthetic oligonucleotide
gcagaggtaatcatgc

SEQ ID NO: 160 - DNA, 16 bp, Artificial sequence
  Synthetic oligonucleotide
gtattatgggctgctg

SEQ ID NO: 161 - DNA, 16 bp, Artificial sequence
  Synthetic oligonucleotide
gatgagaacggtctgc

SEQ ID NO: 162 - DNA, 16 bp, Artificial sequence
  Synthetic oligonucleotide
gcacgacggcccccag

SEQ ID NO: 163 - DNA, 16 bp, Artificial sequence
  Synthetic oligonucleotide
gtaaatctcggcccgg

SEQ ID NO: 164 - DNA, 16 bp, Artificial sequence
  Synthetic oligonucleotide
atctcgacatcaccta

SEQ ID NO: 165 - DNA, 16 bp, Artificial sequence
  Synthetic oligonucleotide
atatatcgccccggcg

SEQ ID NO: 166 - DNA, 16 bp, Artificial sequence
  Synthetic oligonucleotide
actaagaaggtctgct

SEQ ID NO: 167 - DNA, 16 bp, Artificial sequence
  Synthetic oligonucleotide
tgctcggtggaagccc

SEQ ID NO: 168 - DNA, 16 bp, Artificial sequence
  Synthetic oligonucleotide
gtaatagtagcacatt

SEQ ID NO: 169 - DNA, 16 bp, Artificial sequence
  Synthetic oligonucleotide
tggcttaaaacatcag

SEQ ID NO: 170 - DNA, 16 bp, Artificial sequence
  Synthetic oligonucleotide
ttatacagtcagagtg

SEQ ID NO: 171 - DNA, 16 bp, Artificial sequence
  Synthetic oligonucleotide
aaggcatgggtacaat

SEQ ID NO: 172 - DNA, 16 bp, Artificial sequence
  Synthetic oligonucleotide
tcatataccaaggcat

SEQ ID NO: 173 - DNA, 16 bp, Artificial sequence
  Synthetic oligonucleotide
acctattattacctta

SEQ ID NO: 174 - DNA, 16 bp, Artificial sequence
  Synthetic oligonucleotide
gagcggttgctcggtg

SEQ ID NO: 175 - DNA, 16 bp, Artificial sequence
  Synthetic oligonucleotide
agattacaatcccaga

SEQ ID NO: 176 - DNA, 16 bp, Artificial sequence
  Synthetic oligonucleotide
gatctaatatctagtt

SEQ ID NO: 177 - DNA, 16 bp, Artificial sequence
  Synthetic oligonucleotide
attttttacgagggac

SEQ ID NO: 178 - DNA, 16 bp, Artificial sequence
  Synthetic oligonucleotide
gtattgatgtcttccc

SEQ ID NO: 179 - DNA, 16 bp, Artificial sequence
  Synthetic oligonucleotide
tgaacgatgtcctgtg

SEQ ID NO: 180 - DNA, 16 bp, Artificial sequence
  Synthetic oligonucleotide
ccactaatgaaggctg

SEQ ID NO: 181 - DNA, 16 bp, Artificial sequence
  Synthetic oligonucleotide
cttactatgtgaaccc

SEQ ID NO: 182 - DNA, 16 bp, Artificial sequence
  Synthetic oligonucleotide
gttatcgaagatgctg

SEQ ID NO: 183 - DNA, 16 bp, Artificial sequence
  Synthetic oligonucleotide
ttaactaattagctgg

SEQ ID NO: 184 - DNA, 16 bp, Artificial sequence
  Synthetic oligonucleotide
catacctaacaacccc

SEQ ID NO: 185 - DNA, 16 bp, Artificial sequence
  Synthetic oligonucleotide
agatatcgatctgact

SEQ ID NO: 186 - DNA, 16 bp, Artificial sequence
  Synthetic oligonucleotide
atcagtaaacctttac

SEQ ID NO: 187 - DNA, 16 bp, Artificial sequence
  Synthetic oligonucleotide
catacaaatgctccct

SEQ ID NO: 188 - DNA, 16 bp, Artificial sequence
  Synthetic oligonucleotide
agaattaagtagctcc

SEQ ID NO: 189 - DNA, 16 bp, Artificial sequence
  Synthetic oligonucleotide
cacggttattaggtgg

SEQ ID NO: 190 - DNA, 16 bp, Artificial sequence
  Synthetic oligonucleotide
attataataatcccta

SEQ ID NO: 191 - DNA, 16 bp, Artificial sequence
  Synthetic oligonucleotide
agctatataagtttta

SEQ ID NO: 192 - DNA, 16 bp, Artificial sequence
  Synthetic oligonucleotide
acaacgagtatttgga

SEQ ID NO: 193 - DNA, 16 bp, Artificial sequence
  Synthetic oligonucleotide
ggattttagatgtcac

SEQ ID NO: 194 - DNA, 16 bp, Artificial sequence
  Synthetic oligonucleotide
gtaacaaatcacccac

SEQ ID NO: 195 - DNA, 16 bp, Artificial sequence
  Synthetic oligonucleotide
ggagggttaggtctga

SEQ ID NO: 196 - DNA, 16 bp, Artificial sequence
  Synthetic oligonucleotide
ttattgtttagtctcc

SEQ ID NO: 197 - DNA, 16 bp, Artificial sequence
  Synthetic oligonucleotide
atctataagtatagga

SEQ ID NO: 198 - DNA, 16 bp, Artificial sequence
  Synthetic oligonucleotide
gcaatatcatattcta

SEQ ID NO: 199 - DNA, 16 bp, Artificial sequence
  Synthetic oligonucleotide
gatagatcaatcacaa

SEQ ID NO: 200 - DNA, 16 bp, Artificial sequence
  Synthetic oligonucleotide
tttaatctctttagtg

SEQ ID NO: 201 - DNA, 16 bp, Artificial sequence
  Synthetic oligonucleotide
gcagtatacaagaggt

SEQ ID NO: 202 - DNA, 16 bp, Artificial sequence
  Synthetic oligonucleotide
catacccaaatacggc

SEQ ID NO: 203 - DNA, 16 bp, Artificial sequence
  Synthetic oligonucleotide
gtactaatgttgcctt

SEQ ID NO: 204 - DNA, 16 bp, Artificial sequence
  Synthetic oligonucleotide
cctacaaaattggaga

SEQ ID NO: 205 - DNA, 16 bp, Artificial sequence
  Synthetic oligonucleotide
agcatatattaatggt

SEQ ID NO: 206 - DNA, 16 bp, Artificial sequence
  Synthetic oligonucleotide
agattaatgctagagg

SEQ ID NO: 207 - DNA, 16 bp, Artificial sequence
  Synthetic oligonucleotide
ggagatactggccgcc

SEQ ID NO: 208 - DNA, 16 bp, Artificial sequence
  Synthetic oligonucleotide
ggccaatacgccgtca